SEQUENCE LISTING (111 sequences)

SEQ ID NO: 1 - protein, 9 aa, Homo sapiens
TLSQAIVKV

SEQ ID NO: 2 - protein, 10 aa, Homo sapiens
SLIGYSLFSV

SEQ ID NO: 3 - protein, 9 aa, Homo sapiens
SLINPTVIV

SEQ ID NO: 4 - protein, 9 aa, Homo sapiens
KLTAQVEEL

SEQ ID NO: 5 - protein, 9 aa, Homo sapiens
FLAQKCHTL

SEQ ID NO: 6 - protein, 9 aa, Homo sapiens
LLGNFEESV

SEQ ID NO: 7 - protein, 9 aa, Homo sapiens
YLEHYLDNL

SEQ ID NO: 8 - protein, 9 aa, Homo sapiens
IQAGIFQEF

SEQ ID NO: 9 - protein, 10 aa, Homo sapiens
KSAPSTGVVK

SEQ ID NO: 10 - protein, 9 aa, Homo sapiens
STWARSWAY

SEQ ID NO: 11 - protein, 10 aa, Homo sapiens
KSAPSTGRVK

SEQ ID NO: 12 - protein, 9 aa, Homo sapiens
VYKDRLIYF

SEQ ID NO: 13 - protein, 9 aa, Homo sapiens
VYLSAQVQL

SEQ ID NO: 14 - protein, 10 aa, Homo sapiens
SYFEKETLTF

SEQ ID NO: 15 - protein, 9 aa, Homo sapiens
TYNWKGLLF

SEQ ID NO: 16 - protein, 10 aa, Homo sapiens
PWFEGLESRF

SEQ ID NO: 17 - protein, 9 aa, Homo sapiens
RAPETWFEF

SEQ ID NO: 18 - protein, 9 aa, Homo sapiens
YFEKETLTF

SEQ ID NO: 19 - protein, 9 aa, Homo sapiens
MYGSLDSAF

SEQ ID NO: 20 - protein, 10 aa, Homo sapiens
GYMDYAQARF

SEQ ID NO: 21 - protein, 9 aa, Homo sapiens
RYQTMKGLI

SEQ ID NO: 22 - protein, 9 aa, Homo sapiens
RIKTLRRCY

SEQ ID NO: 23 - protein, 9 aa, Homo sapiens
RLRSKRENV

SEQ ID NO: 24 - protein, 9 aa, Homo sapiens
KTRLQARPR

SEQ ID NO: 25 - protein, 9 aa, Homo sapiens
GYMDYAQAR

SEQ ID NO: 26 - protein, 9 aa, Homo sapiens
FLFTSRIRR

SEQ ID NO: 27 - protein, 9 aa, Homo sapiens
CIKTRLQAR

SEQ ID NO: 28 - protein, 9 aa, Homo sapiens
HFLFTSRIR

SEQ ID NO: 29 - protein, 9 aa, Homo sapiens
YTNRLRSKR

SEQ ID NO: 30 - protein, 9 aa, Homo sapiens
TWARSWAYR

SEQ ID NO: 31 - protein, 9 aa, Homo sapiens
YQTMKGLIL

SEQ ID NO: 32 - protein, 9 aa, Homo sapiens
SMLEKTALL

SEQ ID NO: 33 - protein, 9 aa, Homo sapiens
YMYNKVAVL

SEQ ID NO: 34 - protein, 8 aa, Homo sapiens
YLFTFNHL

SEQ ID NO: 35 - protein, 10 aa, Homo sapiens
KSAPSTGGVK

SEQ ID NO: 36 - protein, 10 aa, Homo sapiens
RMSAPSTGGV

SEQ ID NO: 37 - protein, 9 aa, Homo sapiens
ALACDWWFL

SEQ ID NO: 38 - protein, 9 aa, Homo sapiens
ALFGDVKFV

SEQ ID NO: 39 - protein, 9 aa, Homo sapiens
ALLDEVLDV

SEQ ID NO: 40 - protein, 9 aa, Homo sapiens
ALLPCCNRV

SEQ ID NO: 41 - protein, 9 aa, Homo sapiens
ALLYSAFGV

SEQ ID NO: 42 - protein, 9 aa, Homo sapiens
ALMNGLIMT

SEQ ID NO: 43 - protein, 9 aa, Homo sapiens
AMLTVIPEV

SEQ ID NO: 44 - protein, 9 aa, Homo sapiens
AMYSVEITV

SEQ ID NO: 45 - protein, 9 aa, Homo sapiens
CMMISVWNL

SEQ ID NO: 46 - protein, 9 aa, Homo sapiens
FIFEHSYSV

SEQ ID NO: 47 - protein, 9 aa, Homo sapiens
FISEIGPAV

SEQ ID NO: 48 - protein, 9 aa, Homo sapiens
FISSCSLPV

SEQ ID NO: 49 - protein, 9 aa, Homo sapiens
FLALCATGL

SEQ ID NO: 50 - protein, 9 aa, Homo sapiens
FLFERVEGI

SEQ ID NO: 51 - protein, 9 aa, Homo sapiens
FLFGVDEYL

SEQ ID NO: 52 - protein, 9 aa, Homo sapiens
FLGPVIVEI

SEQ ID NO: 53 - protein, 9 aa, Homo sapiens
FLIGFGLWL

SEQ ID NO: 54 - protein, 9 aa, Homo sapiens
FLLDLDPLL

SEQ ID NO: 55 - protein, 9 aa, Homo sapiens
FLLRKVFPL

SEQ ID NO: 56 - protein, 9 aa, Homo sapiens
FLQATDFVV

SEQ ID NO: 57 - protein, 9 aa, Homo sapiens
FLSDLNLLV

SEQ ID NO: 58 - protein, 9 aa, Homo sapiens
FLSDTQVFV

SEQ ID NO: 59 - protein, 9 aa, Homo sapiens
FLSDVKDGV

SEQ ID NO: 60 - protein, 9 aa, Homo sapiens
FLVMYSHFA

SEQ ID NO: 61 - protein, 9 aa, Homo sapiens
FLVSGIAKV

SEQ ID NO: 62 - protein, 9 aa, Homo sapiens
FLYMDYLVL

SEQ ID NO: 63 - protein, 9 aa, Homo sapiens
FQILSVVPV

SEQ ID NO: 64 - protein, 9 aa, Homo sapiens
GLIYFFVQV

SEQ ID NO: 65 - protein, 9 aa, Homo sapiens
GLWEEAYRL

SEQ ID NO: 66 - protein, 9 aa, Homo sapiens
GMLQMDWEV

SEQ ID NO: 67 - protein, 9 aa, Homo sapiens
ILLDCQYLA

SEQ ID NO: 68 - protein, 9 aa, Homo sapiens
IMSAVPFLI

SEQ ID NO: 69 - protein, 9 aa, Homo sapiens
KIIHWPWLV

SEQ ID NO: 70 - protein, 9 aa, Homo sapiens
KLDMGTTLV

SEQ ID NO: 71 - protein, 9 aa, Homo sapiens
KLRSWMYAV

SEQ ID NO: 72 - protein, 9 aa, Homo sapiens
KMLDKLRYV

SEQ ID NO: 73 - protein, 9 aa, Homo sapiens
KMLVDCVPL

SEQ ID NO: 74 - protein, 9 aa, Homo sapiens
KMYKTPIFL

SEQ ID NO: 75 - protein, 9 aa, Homo sapiens
LLFSGCAGL

SEQ ID NO: 76 - protein, 9 aa, Homo sapiens
LLLAIMSAV

SEQ ID NO: 77 - protein, 9 aa, Homo sapiens
LLMVLPFLA

SEQ ID NO: 78 - protein, 9 aa, Homo sapiens
LMNGLIMTV

SEQ ID NO: 79 - protein, 9 aa, Homo sapiens
MLADIPVTI

SEQ ID NO: 80 - protein, 9 aa, Homo sapiens
MLFHSYPPA

SEQ ID NO: 81 - protein, 9 aa, Homo sapiens
MLFNDAIRL

SEQ ID NO: 82 - protein, 9 aa, Homo sapiens
MMKAAMYSV

SEQ ID NO: 83 - protein, 9 aa, Homo sapiens
NLLAEIHGV

SEQ ID NO: 84 - protein, 9 aa, Homo sapiens
QLLDLFYIL

SEQ ID NO: 85 - protein, 9 aa, Homo sapiens
QLLYWFLKV

SEQ ID NO: 86 - protein, 9 aa, Homo sapiens
SLMDKLLPV

SEQ ID NO: 87 - protein, 9 aa, Homo sapiens
TMWDYTIPI

SEQ ID NO: 88 - protein, 9 aa, Homo sapiens
VLWNGIPTA

SEQ ID NO: 89 - protein, 9 aa, Homo sapiens
VLYTIFMKV

SEQ ID NO: 90 - protein, 9 aa, Homo sapiens
VMDNLLIQV

SEQ ID NO: 91 - protein, 9 aa, Homo sapiens
VQWDLLHGV

SEQ ID NO: 92 - protein, 9 aa, Homo sapiens
WLLISVWGL

SEQ ID NO: 93 - protein, 9 aa, Homo sapiens
WMAPEVAAV

SEQ ID NO: 94 - protein, 9 aa, Homo sapiens
WVLELPYFV

SEQ ID NO: 95 - protein, 9 aa, Homo sapiens
YLAAFRFWI

SEQ ID NO: 96 - protein, 9 aa, Homo sapiens
YLAKMSLSV

SEQ ID NO: 97 - protein, 9 aa, Homo sapiens
YLDGIITIV

SEQ ID NO: 98 - protein, 9 aa, Homo sapiens
YLFNSVVNV

SEQ ID NO: 99 - protein, 9 aa, Homo sapiens
YLFVQPDYI

SEQ ID NO: 100 - protein, 9 aa, Homo sapiens
YLLMVLPFL

SEQ ID NO: 101 - protein, 9 aa, Homo sapiens
YLMGVGALA

SEQ ID NO: 102 - protein, 9 aa, Homo sapiens
YLQAYSATV

SEQ ID NO: 103 - protein, 9 aa, Homo sapiens
YLSTIVTEV

SEQ ID NO: 104 - protein, 9 aa, Homo sapiens
YLWFLCRYL

SEQ ID NO: 105 - protein, 9 aa, Homo sapiens
YLWLIYCYL

SEQ ID NO: 106 - protein, 9 aa, Homo sapiens
YMDYLVLVL

SEQ ID NO: 107 - protein, 9 aa, Homo sapiens
YMRCCLWKL

SEQ ID NO: 108 - DNA, 35 bp, synthetic construct
  source 1..35: mol_type = other DNA
gtctgtgtgctgcaggagctgagctgactctgctg

SEQ ID NO: 109 - DNA, 33 bp, synthetic construct
  source 1..33: mol_type = other DNA
gatacgcgtatatctggcccgtacatcgcatcg

SEQ ID NO: 110 - DNA, 25 bp, synthetic construct
  source 1..25: mol_type = other DNA
gcacgttctccacgtatgcggcgtg

SEQ ID NO: 111 - DNA, 19 bp, synthetic construct
  source 1..19: mol_type = other DNA
tttcttcacccttccagta

(19) United States
(12) Patent Application Publication
(10) Pub. No.: US 2025/0352576 A1
(43) Pub. Date: Nov. 20, 2025

(54) METHODS AND COMPOSITIONS FOR TREATING GLIOMAS

(71) Applicants: THE REGENTS OF THE UNIVERSITY OF CALIFORNIA, Oakland, CA (US); THE CHILDREN'S HOSPITAL OF PHILADELPHIA, Philadelphia, PA (US)

(72) Inventors: Anthony WANG, Los Angeles, CA (US); Robert PRINS, Pacific Palisades, CA (US); Linda LIAU, Los Angeles, CA (US); Geoffrey OWENS, Woodland Hills, CA (US); Yi XING, Philadelphia, PA (US); David NATHANSON, Los Angeles, CA (US)

(73) Assignees: THE REGENTS OF THE UNIVERSITY OF CALIFORNIA, Oakland, CA (US); THE CHILDREN'S HOSPITAL OF PHILADELPHIA, Philadelphia, PA (US)

(21) Appl. No.: 18/853,032

(22) PCT Filed: Mar. 31, 2023
(86) PCT No.: PCT/US2023/017178

§ 371 Date: Sep. 30, 2024

Related U.S. Application Data
(60) Provisional application No. 63/326,408, filed on Apr. 1, 2022.

(51) Int. Cl. A61K 35/15; A61K 38/00; A61K 40/19; A61K 40/42; A61P 35/00; C07K 7/06; C07K 14/74; C07K 16/28; C12N 5/0783; C12N 5/0784; G01N 33/569; G01N 33/574

(52) U.S. Cl. CPC A61K 35/15; A61K 40/19; A61K 40/421; A61P 35/00; C07K 7/06; C07K 14/70539; C07K 16/2818; C12N 5/0636; C12N 5/0639; G01N 33/56972; G01N 33/574; A61K 38/00; C07K 2319/00; G01N 2333/70539

(57) ABSTRACT

The current disclosure fulfills a need in the art by providing methods and compositions for treating and vaccinating individuals against cancer. The disclosure describes isolated peptides comprising at least 70% sequence identity to a peptide of one of SEQ ID NOS: 1-107. The peptide may comprise at least 6 contiguous amino acids of a peptide of one of SEQ ID NOS: 1-107. The disclosure also describes a peptide comprising at least 6 contiguous amino acids from a peptide of one of SEQ ID NOS: 1-107, wherein the peptide comprises an alternative splice site junction. Also described is a polypeptide comprising the peptide, pharmaceutical compositions comprising the isolated peptide, nucleic acids encoding the peptide, and expression vectors and host cells comprising the nucleic acids of the disclosure. The nucleic acids of the disclosure include nucleic acids that are RNA or DNA. Also provided is an in vitro isolated dendritic cell comprising a peptide, nucleic acid, or expression vector of the disclosure.

### STATEMENT OF GOVERNMENT SUPPORT

This invention was made with government support under CA222695 and CA211015, awarded by the National Institutes of Health. The government has certain rights in the invention.

This application claims priority of U.S. Provisional Patent Application No. 63/326,408, filed Apr. 1, 2022, which is hereby incorporated by reference in its entirety.

The application contains a Sequence Listing in compliance with ST.26 format and is hereby incorporated by reference in its entirety. Said Sequence Listing, created on Mar. 27, 2023 is named UCLAP0122WO.xml and is 96,141 bytes in size.

### BACKGROUND OF THE INVENTION

### Field of the Invention

This invention relates to the field of treatment of cancer.

### Background

High-grade gliomas (HGG) are among the most lethal of human cancers; median overall survival is typically under 2 years from initial diagnosis, even with optimal multi-modal therapy. Recurrence is universal, as tumors invade and infiltrate the surrounding brain, making complete surgical excision impossible, while quiescent tumor-initiating cell populations evade adjuvant treatments. Various forms of immunotherapy, including checkpoint blockade and active vaccination modalities, have both shown promise in addressing the issue of recurrence in pre-clinical studies. In general, HGG are relatively low in somatic mutational burden, generally a negative predictor of response to immunotherapy, though potentially not in high-grade gliomas. The subtypes of HGG found in adults, and consequently the subtypes that have been more frequently studied in the context of clinical trials of immunotherapy, are dramatically heterogeneous and often involve copy number variability, without highly-conserved truncal mutations suitable for immune targeting. There are reasons to believe that the juvenile forms of HGG will respond differently to immunotherapy than adult forms of HGG, as they consistently carry a small number of oncogenic driver mutations.

Juvenile presentation of HGG has recently been shown to frequently involve somatic mutations of the H3-3A gene, which encodes the histone H3 variants. Missense mutations at codons K27 or G34 have divergent effects on gene expression that is context-dependent. H3G34R/V HGG display distinct characteristics that differentiate it from the better-characterized H3K27M HGG. H3G34R/V HGG most commonly occur later in childhood, and is typically cortical and lobar in location, in contrast with the midline or diencephalic location of H3K27M HGG. Concurrent TP53, ATRX/DAXX, and PDGFRA alterations are frequent in H3G34R/V HGGs. Profiling of resected H3G34R/V tumors has revealed that they are relatively devoid of infiltrating immune cells, indicating that there may be 1) few immunogenic tumor-specific antigens (TSAs), 2) inhibited trafficking of cytotoxic T lymphocytes (CTLs) into the tumor microenvironment, and 3) immune-evasive characteristics of HGG within the tumor microenvironment. Therefore, there is a need in the art to identify TSAs that can be used as targets to treat patients having gliomas.

### SUMMARY OF THE INVENTION

The current disclosure fulfills a need in the art by providing methods and compositions for treating and vaccinating individuals against cancer. The disclosure describes isolated peptides comprising at least 70% sequence identity to a peptide of one of SEQ ID NOS: 1-107. The peptide may comprise at least 6 contiguous amino acids of a peptide of one of SEQ ID NOS: 1-107. The disclosure also describes a peptide comprising at least 6 contiguous amino acids from a peptide of one of SEQ ID NOS: 1-107, wherein the peptide comprises an alternative splice site junction. Also described is a polypeptide comprising the peptide, pharmaceutical compositions comprising the isolated peptide, nucleic acids encoding the peptide, and expression vectors and host cells comprising the nucleic acids of the disclosure. The nucleic acids of the disclosure include nucleic acids that are RNA or DNA. Also provided is an in vitro isolated dendritic cell comprising a peptide, nucleic acid, or expression vector of the disclosure.

Methods include a method of making a cell comprising contacting the a cell with a peptide or polypeptide of the disclosure or comprising transferring a nucleic acid or expression vector of the disclosure into a cell, such as a host cell, dendritic cell, or macrophage. The cell may be further described as a mature dendritic cell. The cell may comprise a monocyte-derived dendritic or macrophage cell. Also described is a method of making a peptide or polypeptide comprising transferring a nucleic acid or expression vector of the disclosure into a cell. The method may further comprise isolating the expressed peptide or polypeptide. Methods also include a method of producing glioma-specific immune effector cells comprising: (a) obtaining a starting population of immune effector cells; and (b) contacting the starting population of immune effector cells with a peptide or polypeptide of the disclosure, thereby generating peptide-specific immune effector cells.

The disclosure also describes peptide-specific engineered T cells produced according to the methods of the disclosure and pharmaceutical compositions comprising the engineered T cells. Also described is a method of treating or preventing glioma in a subject, the method comprising administering an effective amount of a peptide, polypeptide, pharmaceutical composition, nucleic acid, dendritic cell, or peptide-specific T cell of the disclosure. Methods may include reducing the risk of recurrence of the glioma; reducing the risk of progression; and/or increasing the chance of progression-free survival, relapse-free survival, and/or recurrence-free survival in a subject in need thereof by administering an effective amount of a peptide, polypeptide, pharmaceutical composition, nucleic acid, dendritic cell, or peptide-specific T cell of the disclosure. Methods include a method of cloning a peptide-specific T cell receptor (TCR), the method comprising (a) obtaining a starting population of immune effector cells; (b) contacting the starting population of immune effector cells with the peptide or polypeptide of the disclosure, thereby generating peptide-specific immune effector cells; (c) purifying immune effector cells specific to the peptide, and (d) isolating a TCR sequence from the purified immune effector cells. Also provide is a method for prognosing a patient or for detecting T cell responses in a patient, the method comprising: contacting a biological sample from the patient with a peptide or polypeptide of the disclosure.

Also described is a composition comprising at least one MHC polypeptide and a peptide of the disclosure and peptide-specific binding molecule that bind to a peptide of the disclosure or that bind to a peptide-MHC complex. Exemplary binding molecules include antibodies, TCR mimic antibodies, scFvs, nanobodies, camellids, aptamers, and DARPINs. Related methods provide for a method comprising contacting a composition comprising at least one MHC polypeptide and a peptide of the disclosure with a composition comprising T cells and detecting T cells with bound peptide and/or MHC polypeptide by detecting a detection tag. Also described are kits comprising a peptide, nucleic acid, expression vector, or composition of the disclosure.

The peptide may be 9 amino acids in length or shorter. The peptide may have at least, at most, exactly, or consists of 5, 6, 7, 8, 9, 10, 11, 12, 13, 14, or 15 amino acids (or any range derivable therein). The peptide may consist of 9 amino acids. The peptide may be further defined as being immunogenic. The term immunogenic may refer to the production of an immune response, such as a protective immune response. The peptide may be modified. The modification may comprise conjugation to a molecule. The molecule may be an antibody, a lipid, an adjuvant, or a detection moiety (tag). The peptide may comprise 100% sequence identity to a peptide of one of SEQ ID NOS: 1-107. Peptides of the disclosure also include those that have at least 90% sequence identity to a peptide of one of SEQ ID NOS: 1-107. The peptides of the disclosure may have 1, 2, or 3 substitutions relative to a peptide of one of SEQ ID NOS: 1-107. The peptide may have at least or at most 1, 2, 3, 4, or 5 substitutions relative to a peptide of one of SEQ ID NOS: 1-107.

The pharmaceutical composition may be formulated for parenteral administration, intravenous injection, intramuscular injection, inhalation, or subcutaneous injection. The peptide may be comprised in a liposome, lipid-containing nanoparticle, or in a lipid-based carrier. The pharmaceutical preparation may be formulated for injection or inhalation as a nasal spray. The compositions of the disclosure may be formulated as a vaccine. The vaccine may comprise a mRNA vaccine. The composition may comprise at least two peptides of the disclosure. The composition may comprise, comprise at least, or comprise at most 1, 2, 3, 4, 5, 6, 7, 8, 9, 10, 11, 12, 14, or 15 peptides of the disclosure. The composition may further comprises an adjuvant.

The dendritic cell may comprise a mature dendritic cell. The dendritic cells may have peptides of the disclosure on the surface of the dendritic cell, wherein the peptide is complexed with a HLA and/or the dendritic cells may comprise nucleic acids encoding for peptides of the disclosure. The dendritic cell may comprise at least two peptides of the disclosure. The dendritic cell may comprise, comprise at least, or comprise at most 1, 2, 3, 4, 5, 6, 7, 8, 9, 10, 11, 12, 13, 14, or 15 peptides of the disclosure or nucleic acids encoding a peptide of the disclosure. The dendritic cell may comprise the peptides on the surface of the cell as a pMHC complex. The cell may be a cell with an HLA-A type. The HLA may be a HLA-A, HLA-B, or HLA-C. The cell may be a HLA-A02 or HLA-A03 type. The cell may be an HLA-A01, HLA-A02, HLA-A24, HLA-B07, HLA-B08, HLA-B15, or HLA-B40. The method may further comprise isolating the expressed peptide or polypeptide. The T cell may comprise a CD8⁺ T cell. The T cell may be a CD4⁺ T cell, a Th1, Th2, Th17, Th9, or Tfh T cell, a cytotoxic T cell, a memory T cell, a central memory T cell, or an effector memory T cell.

In the methods of the disclosure, contacting may be further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface. The APCs may be dendritic cells.

The immune effector cells may be T cells, peripheral blood lymphocytes, natural killer (NK) cells, invariant NK cells, or NKT cells. The immune effector cells may be ones that have been differentiated from mesenchymal stem cell (MSC) or induced pluripotent stem (iPS) cells. The T cell may include T cells that are further defined as CD8⁺ T cells, CD4⁺ T cells, or γδ T cells. The T cells may be further classified as cytotoxic T lymphocytes (CTLs). The compositions of the disclosure may comprise a peptide of SEQ ID NO:32, 33, and/or 34. The immunogenic peptides in the composition may consist of SEQ ID NO:32, 33, and 34. The compositions of the disclosure may comprise a peptide of SEQ ID NO:32, 33, and/or 39. The immunogenic peptides in the composition may consist of SEQ ID NO:32, 33, and 39.

The subject described in the methods of the disclosure may be a human. The subject may be a laboratory animal. The subject may be a mouse, rat, pig, horse, rabbit, or guinea pig. The methods may further comprise further administration of at least a second therapeutic agent. The second therapeutic agent may be an additional therapy described herein. The second therapeutic agent may be an anti-cancer agent. The anti-cancer treatment may comprise one or more of surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy. The anti-cancer treatment may comprise an immunotherapy. The immunotherapy may comprise immune checkpoint immunotherapy (ICI) therapy. The ICI therapy may comprise a monotherapy or a combination ICI therapy. The ICI therapy may comprise an inhibitor of PD-1, PDL1, PDL2, CTLA-4, B7-1, B7-2, and combinations thereof. The ICI therapy may comprise anti-PD1 monoclonal antibody monotherapy. The ICI therapy may comprise an anti-PD-1 monoclonal antibody, an anti-CTLA-4 monoclonal antibody, and combinations thereof. The ICI therapy may comprise one or more of nivolumab, pembrolizumab, pidilizumab, ipilimumab or tremelimumab.

The composition of the disclosure may comprise a MHC polypeptide and a peptide of the disclosure and wherein the MHC polypeptide and/or peptide is conjugated to a detection tag. As such, suitable detection tags include, but are not limited to radioisotopes, fluorochromes, chemiluminescent compounds, dyes, and proteins, including enzymes. The tag may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent tags that produce signals include, but are not limited to bioluminescence and chemiluminescence. Examples of suitable fluorescent tags include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.). Detection tags also include streptavidin or it's binding partner, biotin.

The MHC polypeptide and peptide may be operatively linked. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. The MHC polypeptide and peptide may be operatively linked through a peptide bond. The MHC polypeptide and peptide may be operatively linked through van der Waals forces. The peptide-MHC may be operatively linked to form a pMHC complex. At least two pMHC complexes may be operatively linked together. Exactly, at least, or at most 2, 3, 4, 5, 6, 7, 8, 9, or 10 pMHC complexes may be operatively linked to each other. At least two MHC polypeptides may be linked to one peptide. The average ratio of MHC polypeptides to peptides may be 1:1 to 4:1. The ratio or average ratio may be at least, at most, or about 1, 2, 3, 4, 5, or 6 to about 1, 2, 3, 4, 5, or 6 (or any derivable range therein).

The peptide may be complexed with MHC. The MHC may comprise HLA-A type. The MHC may be further defined as HLA-A3 or HLA-All type. The peptides may be loaded onto dendritic cells, lymphoblastoid cells, peripheral blood mononuclear cells (PBMCs), artificial antigen presentation cells (aAPC) or artificial antigen presenting surfaces. The artificial antigen presenting surface may comprise a MHC polypeptide conjugated or linked to a surface. Exemplary surfaces include a bead, microplate, glass slide, or cell culture plate.

Method of the disclosure may further comprise counting the number of T cells bound with peptide and/or MHC. The composition comprising T cells may be isolated from a patient having or suspected of having cancer. The cancer may comprise a peptide-specific cancer, wherein the peptide of one of SEQ ID NOS: 1-107 or a peptide of the disclosure. The subject may be a subject that has been diagnosed and/or determined to have a cancer such as glioma. The subject or patient may also be one that has been characterized as having a peptide-specific glioma, such as a peptide of the disclosure or a peptide of one of SEQ ID NOS: 1-107. The method may further comprise sorting the number of T cells bound with peptide and/or MHC. Methods of the disclosure may also comprise or further comprise sequencing one or more TCR genes from T cells bound with peptide and/or MHC. The method may comprise or further comprise sequencing the TCR alpha and/or beta gene(s) from a TCR, such as a TCR that binds to a peptide of the disclosure. Methods may also comprise or further comprise grouping of lymphocyte interactions by paratope hotspots (GLIPH) analysis. This is further described in Glanville et al., Nature. 2017 Jul. 6; 547 (7661): 94-98, which is herein incorporated by reference.

The compositions of the disclosure may be serum-free, mycoplasma-free, endotoxin-free, and/or sterile. The methods may further comprise culturing cells of the disclosure in media, incubating the cells at conditions that allow for the division of the cell, screening the cells, and/or freezing the cells. The method may further comprise isolating the expressed peptide or polypeptide from a cell of the disclosure.

Methods of the disclosure may comprise or further comprise screening the dendritic cell for one or more cellular properties. The method may further comprise contacting the cell with one or more cytokines or growth factors. The one or more cytokines or growth factors may comprise GM-CSF. The cellular property may comprise cell surface expression of one or more of CD86, HLA, and CD14. The dendritic cell may be derived from a CD34+ hematopoietic stem or progenitor cell.

The contacting in the methods of the disclosure may be further defined as co-culturing the starting population of immune effector cells with antigen presenting cells (APCs), wherein the APCs present the peptide on their surface. The APCs may be dendritic cells. The dendritic cell may be derived from a peripheral blood monocyte (PBMC). The dendritic cells may be isolated from PBMCs. The dendritic cells may be cells in which the DCs are derived from are isolated by leukaphereses.

Peptide-MHC (pMHC) complexes may be made by contacting a peptide of the disclosure with a MHC complex. The peptide may be expressed in the cell and binds to endogenous MHC complex to form a pMHC. Peptide exchange may be used to make the pMHC complex. For example, cleavable peptides, such as photocleavable peptides may be designed that bind to and stabilize the MHC. Cleavage of the peptide (eg. by irradiation for photocleavable peptides) dissociates the peptide from the HLA complex and results in an empty HLA complex that disintegrates rapidly, unless UV exposure is performed in the presence of a “rescue peptide.” Thus, the peptides of the disclosure may be used as “rescue peptides” in the peptide exchange procedure. The disclosure also describes pMHC complexes comprising a peptide of the disclosure. The pMHC complex may be operatively linked to a solid support or may be attached to a detectable moiety, such as a fluorescent molecule, a radioisotope, or an antibody. Also described is a peptide-MHC multimeric complexes that include, include at least or include at most 1, 2, 3, 4, 5, or 6 peptide-MHC molecules operatively linked together. The linkage may be covalent, such as through a peptide bond, or non-covalent. pMHC molecules may be bound to a biotin molecule. Such pMHC molecules may be multimerized through binding to a streptavidin molecule. pMHC multimers may be used to detect antigen-specific T cells or TCR molecules that are in a composition or in a tissue. The multimers may be used to detect peptide-specific T cells in situ or in a biopsy sample. Multimers may be bound to a solid support or deposited on a solid support, such as an array or slide. Cells may then be added to the slide, and detection of the binding between the pMHC multimer and cell may be conducted. Accordingly, the pMHC molecules and multimers of the disclosure may be used to detect and diagnose cancer in subjects or to determine immune responses in individuals with cancer.

Obtaining, as defined in the methods described herein, may comprise isolating the starting population of immune effector cells from peripheral blood mononuclear cells (PBMCs). The starting population of immune effector cells may be obtained from a subject. The subject may be one that has a glioma, such as a peptide-specific glioma. The subject may be one that has been determined to have a glioma that expresses a peptide of the disclosure. The glioma may comprise high-grade glioma. The glioma may also be defined as one that has mutations of the H3-3A gene, such as somatic mutations of the H3-3A gene. The subject may be one that has been determined to have high grade glioma and/or glioma with mutations of the H3-3A gene. The glioma may be further defined as and/or the subject has been determined to have a glioma with a H3G34R/V and/or H3K27M mutation in the histone H3 gene. The mutation may comprise the H3G34R/V. The mutation may comprise the H3K27M mutation. In some aspects, either the H3G34R/V and/or H3K27M mutation is excluded. The methods of the disclosure may comprise or further comprise introducing the peptides or a nucleic acid encoding the peptide into the dendritic cells prior to the co-culturing. The introduction of the peptide may be done by transfecting or infecting dendritic cells with a nucleic acid encoding the peptide or by incubating the peptide with the dendritic cells. The peptide or nucleic acids encoding the peptide may be introduced by electroporation. Other methods of transfer of nucleic acids are known in the art, such as lipofection, calcium phosphate transfection, transfection with DEAE-dextran, microinjection, and virus-mediated transduction. The peptide or nucleic acids encoding the peptide may be introduced by adding the peptide or nucleic acid encoding the peptide to the dendritic cell culture media. The immune effector cells may be co-cultured with a second population of dendritic cells into which the peptide or the nucleic acid encoding the peptide has been introduced. A population of CD4-positive or CD8-positive and peptide MHC tetramer-positive T cells may be purified from the immune effector cells following the co-culturing. The population of CD4-positive or CD8-positive and peptide MHC tetramer-positive T cells may be purified by fluorescence activated cell sorting (FACS). A clonal population of peptide-specific immune effector cells may be generated by limiting or serial dilution followed by expansion of individual clones by a rapid expansion protocol.

Purifying may further comprise generation of a clonal population of peptide-specific immune effector cells by limiting or serial dilution of sorted cells followed by expansion of individual clones by a rapid expansion protocol. Methods of the disclosure may comprise or further comprise cloning of a T cell receptor (TCR) from the clonal population of peptide-specific immune effector cells. The term isolating in the methods of the disclosure may be defined as cloning of a T cell receptor (TCR) from the clonal population of peptide-specific immune effector cells. Cloning of the TCR may be cloning of a TCR alpha and a beta chain. The TCR may be cloned using a 5′-Rapid amplification of cDNA ends (RACE) method. The TCR alpha and beta chains may be cloned using a 5′-Rapid amplification of cDNA ends (RACE) method. The cloned TCR may be subcloned into an expression vector. The expression vector may comprise a linker domain between the TCR alpha sequence and TCR beta sequence. The expression vector may be a retroviral or lentiviral vector. The vector may also be an expression vector described herein. The linker domain may comprise a sequence encoding one or more peptide cleavage sites. The one or more cleavage sites may be a Furin cleavage site and/or a P2A cleavage site. The TCR alpha sequence and TCR beta sequence may be linked by an IRES sequence.

A host cell of the disclosure may be transduced with an expression vector to generate an engineered cell that expresses the TCR alpha and/or beta chains. The host cell may be an immune cell. The immune cell may be a T cell and the engineered cell may be referred to as an engineered T cell. The T cell may be type of T cell described herein, such as a CD8⁺ T cell, CD4⁺ T cell, or γδ T cell. The starting population of immune effector cells may be obtained from a subject having a cancer or a peptide-specific cancer and the host cell may be allogeneic or autologous to the subject. The peptide-specific T cells may be autologous or allogeneic. A population of CD4-positive or CD8-positive and peptide MHC tetramer-positive engineered T cells may be purified from the transduced host cells. A clonal population of peptide-specific engineered T cells may be generated by limiting or serial dilution followed by expansion of individual clones by a rapid expansion protocol. Purifying in the methods of the disclosure may be defined as purifying a population of CD4-positive or CD8-positive and peptide MHC tetramer-positive T cells from the immune effector cells following the co-culturing.

The peptide may be linked to a solid support. The peptide may be conjugated to the solid support or is bound to an antibody that is conjugated to the solid support. The solid support may comprise a microplate, a bead, a glass surface, a slide, or a cell culture dish. The solid support may comprise a nanofluidic chip. Detecting T cell responses may comprise detecting the binding of the peptide to the T cell or TCR. Detecting T cell responses may comprise an ELISA, ELISPOT, or a tetramer assay.

Kits of the disclosure may comprise a peptide of the disclosure in a container. The peptide may be comprised in a pharmaceutical preparation. The pharmaceutical preparation may be formulated for parenteral administration or inhalation. The peptide may be comprised in a cell culture media.

“Treatment” or treating may refer to any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. The treatment may exclude prevention of the disease.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-D. The H3G34R tumor model. (A) Chromatograms showing the presence of the G34R mutation in three independently derived H3G34R tumor lines. The G to A transition results in a T on the opposite strand (highlighted in grey). (B) Bar graph showing similar levels of expression in the three H3G34R RCAS lines of genes expressed by neural progenitors associated with multipotency (FoxG1, Hes5, Hey1, Nes, Oligo2, Sox2), and determination of neuronal (Asc11/Mash1, Gsx1, Hes6, Sox4), or oligodendrocyte fate (Olig1, Pdgfra, Sox10). (C) Chemiluminescence signal in the brains of mice implanted with H3G34R RCAS2 cells (D) Hematoxylin/eosin-stained coronal section of cerebral cortex showing the development of a large tumor mass after implantation of H3G34R RCAS2 cells. Scale bar=250 microns. The nucleotide sequence shown in FIG. 1A is SEQ ID NO:111.

FIG. 2A-C. DC vaccination provides survival benefit (A) Schematic outline of the protocol used to treat animals after orthotopic placement of H3G34R RCAS2 tumor cells. (B) Log transformed average radiance values (±standard error) of 10 mice per group plotted against time after implanting tumor cells, and showing a divergence between treated and untreated animals beginning around 28 days. (C) Kaplan-Meier plot showing a significant survival benefit over time after DC vaccination (p=0.0447, Gehan-Breslow-Wilcoxon test).

FIG. 3. Predicted affinities of candidate TSAs identified by IRIS. Peptides were selected that bound to HLA-A*0201 with a calculated Kd of <100 nM. Heat map constructed from 1/Kd values and scaled to the highest value in each row showing that candidate peptides are predicted to bind to other HLA-A alleles and, in some cases, to HLA-B and -C alleles. The three peptides predicted to bind to H2-K^{b} are shown in red. The sequences shown in FIG. 3 correspond to SEQ ID NOS: 37-86, 32, 87-98, 39, 99-107, and 33, respectively.

| Sequence | SEQ ID NO: |
|---|---|
| ALACDWWFL | 37 |
| ALFGDVKFV | 38 |
| ALLDEVLDV | 39 |
| ALLPCCNRV | 40 |
| ALLYSAFGV | 41 |
| ALMNGLIMT | 42 |
| AMLTVIPEV | 43 |
| AMYSVEITV | 44 |
| CMMISVWNL | 45 |
| FIFEHSYSV | 46 |
| FISEIGPAV | 47 |
| FISSCSLPV | 48 |
| FLALCATGL | 49 |
| FLFERVEGI | 50 |
| FLFGVDEYL | 51 |
| FLGPVIVEI | 52 |
| FLIGFGLWL | 53 |
| FLLDLDPLL | 54 |
| FLLRKVFPL | 55 |
| FLQATDFVV | 56 |
| FLSDLNLLV | 57 |
| FLSDTQVFV | 58 |
| FLSDVKDGV | 59 |
| FLVMYSHFA | 60 |
| FLVSGIAKV | 61 |
| FLYMDYLVL | 62 |
| FQILSVVPV | 63 |
| GLIYFFVQV | 64 |
| GLWEEAYRL | 65 |
| GMLQMDWEV | 66 |
| ILLDCQYLA | 67 |
| IMSAVPFLI | 68 |
| KIIHWPWLV | 69 |
| KLDMGTTLV | 70 |
| KLRSWMYAV | 71 |
| KMLDKLRYV | 72 |
| KMLVDCVPL | 73 |
| KMYKTPIFL | 74 |
| LLFSGCAGL | 75 |
| LLLAIMSAV | 76 |
| LLMVLPFLA | 77 |
| LMNGLIMTV | 78 |
| MLADIPVTI | 79 |
| MLFHSYPPA | 80 |
| MLFNDAIRL | 81 |
| MMKAAMYSV | 82 |
| NLLAEIHGV | 83 |
| QLLDLFYIL | 84 |
| QLLYWFLKV | 85 |
| SLMDKLLPV | 86 |
| SMLEKTALL | 32 |
| TMWDYTIPI | 87 |
| VLWNGIPTA | 88 |
| VLYTIFMKV | 89 |
| VMDNLLIQV | 90 |
| VQWDLLHGV | 91 |
| WLLISVWGL | 92 |
| WMAPEVAAV | 93 |
| WVLELPYFV | 94 |
| YLAAFRFWI | 95 |
| YLAKMSLSV | 96 |
| YLDGIITIV | 97 |
| YLFNSVVNV | 98 |
| YLFTFLNHL | 39 |
| YLFVQPDYI | 99 |
| YLLMVLPFL | 100 |
| YLMGVGALA | 101 |
| YLQAYSATV | 102 |
| YLSTIVTEV | 103 |
| YLWFLCRYL | 104 |
| YLWLIYCYL | 105 |
| YMDYLVLVL | 106 |
| YMRCCLWKL | 107 |
| YMYNKVAVL | 33 |

FIG. 4. Candidate peptides bind HLA-A*0201. HLA stabilization assay confirms that the three selected peptides stabilize cell surface HLA-A*0201 on TAP deficient T2 cells. Bar graph shows the percent increase in HLA expression after incubation with peptides (mean fluorescence intensity (MFI)^{peptide treated cells}-MFI^{untreated cells}/MFI^{untreated cells}×100). A peptide spanning the H3K27M mutation was used a positive control (RMSAPSTGGV—SEQ ID NO: 36). The sequences shown in FIG. 4 correspond to SEQ ID NOS: 32, 33, 39, and 36, respectively.

FIG. 5A-B. Peptide-pulsed DC vaccination increases survival. Mice were treated with DCs pulsed with either the candidate therapeutic peptides (SMLEKTALL—SEQ ID NO: 32, YMYNKVAVL—SEQ ID NO:33, YLFTFNHL—SEQ ID NO:34), or negative control peptides (KSAPSTGRVK—SEQ ID NO:11, KSAPSTGVVK—SEQ ID NO:9, KSAPSTGGVK—SEQ ID NO:35). (A) Log transformed average radiance values (±standard error) of 15 mice per group plotted against time after implanting tumor cells. Arrows indicate the days DCs were injected (p=0.047, Mann-Whitney test). (B) Kaplan-Meier plot showing a significant survival benefit over time after DC vaccination with SYY peptides (p=0.0275, Gehan-Breslow-Wilcoxon test).

FIG. 6A-D. Analysis of TILs after DC vaccination. Seven days after the second DC vaccination TILs were isolated from four animals; cells from two animals treated with the SYY peptides were pooled, and cells from two animals treated with control NC peptides were combined, and stained with two antibody panels. (A and C) The relative number of T cells in TILs from the animals treated with the SYY peptides is greater than in TILs from animals treated with the control NC peptides; many of these T cells are PD-1⁺ CD8⁺. (B and D) The CD11b⁺ myeloid fraction comprised predominantly Ly6C⁺ CD11c⁺ monocyte-derived DCs or macrophages; the relative number of XCR1⁺ monocyte-derived DCs was higher in the TILs from animals treated with the SYY ppDCs.

FIG. 7. Paucity of CD8 T cells in untreated H3.3 G34R tumors.

FIG. 8. Relative expression of PD1 and CD25 by CD4 T cells in TIL fractions from animals treated with the SYY and G34 peptides.

FIG. 9. Relative expression of F4/80 by monocyte-derived cells in TIL fractions from animals treated with the SYY and G34 peptides.

FIG. 10A-D. The RCAS/H3G34R tumor model. (A) Chromatograms showing the presence of the G34R mutation in three independently-derived RCAS/H3G34R tumor lines. The G to A transition results in a T on the opposite strand (highlighted in grey). (B) Stacked bar graph showing similar levels of expression in the three RCAS/H3G34R lines of genes expressed by neural progenitors associated with multipotency (FoxG1, Hes5, Hey1, Nes, Oligo2, Sox2), and determination of neuronal (Ascl1/Mash1, Gsx1, Hes6, Sox4), or oligodendrocyte fate (Olig1, Pdgfra, Sox10). (C) Chemiluminescence signal in the brains of mice implanted with RCAS/H3G34R cells (mean±SEM). (D) Hematoxylin/cosin-stained coronal section of cerebral cortex showing the development of a large tumor mass after implantation of RCAS/H3G34R cells. Scale bar=250 microns. Inset shows position of the tumor in a whole brain coronal section. Scale bar=1 mm. Hematoxylin/cosin-stained coronal section of cerebral cortex showing the development of a large tumor mass after implantation of RCAS/H3G34R cells. Scale bar=250 microns. The nucleotide sequence shown in FIG. 10A is SEQ ID NO:111.

FIG. 11A-B. DC vaccination provides survival benefit (A) Schematic outline of the protocol used to treat animals after orthotopic placement of RCAS/H3G34R tumor cells. (B) Kaplan-Meier plot showing a significant survival benefit over time after DC vaccination combined with checkpoint blockade (p=0.0187, Mantel-Cox test).

FIG. 12. Increased CD8⁺ T cells in TIL fractions from ppDC-treated animals. Seven days after the second DC vaccination, TILs were isolated from brains from treated and untreated animals (two animals per group) and analyzed by flow cytometry. TILs were stained with a panel comprising CD45, CD3, CD4, CD8, CD11b, and Ly6C antibodies.

FIG. 13A-C. Higher density of T cells and myeloid cells in sections of brain tumor from an animal treated with DC/peptide. (A and B) Sections showing the extent of neovascularization visualized by CD31 immunostaining (false-colored white). The higher density of DAPI-stained nuclei (blue) demarcates the tumor mass within the section. Scale bar=200 microns. Insets show T cells and myeloid cells in close association with blood vessels located within the tumors. CD8 (turquoise), CD4 (yellow), CD11b/ITGAM (red), CD31/PECAM-1 (false-colored white). Scale bar=25 microns. (C) Quantification of the density of endothelial cells (CD31), myeloid cells (CD11b), and T cells (CD4, CD8) in the sections of tumor.

FIG. 14A-B. Peptide-pulsed DC vaccination increases survival. Mice were treated with DCs pulsed with either the candidate therapeutic peptides (SMLEKTALL—SEQ ID NO: 32, YMYNKVAVL—SEQ ID NO:33, YLFTFNHL—SEQ ID NO:34), or negative control (NC) peptides (KSAPSTGRVK—SEQ ID NO:11, KSAPSTGVVK—SEQ ID NO:9, KSAPSTGGVK—SEQ ID NO:35). (A) Log transformed average radiance values (±standard error) of 15 mice per group plotted against time after implanting tumor cells. Arrows indicate the days DCs were injected (p=0.047, Mann-Whitney test). (B) Kaplan-Meier plot showing a significant survival benefit over time after SYY ppDC vaccination (p=0.0232, Mantel-Cox test).

### DETAILED DESCRIPTION OF THE INVENTION

High-grade gliomas (HGG) are among the most lethal of human cancers; median overall survival is typically under 2 years from initial diagnosis, even with optimal multi-modal therapy. Recurrence is universal, as tumors invade and infiltrate the surrounding brain, making complete surgical excision impossible, while quiescent tumor-initiating cell populations evade adjuvant treatments. Various forms of immunotherapy, including checkpoint blockade and active vaccination modalities, have both shown promise in addressing the issue of recurrence in pre-clinical studies. In general, HGG are relatively low in somatic mutational burden, generally a negative predictor of response to immunotherapy, though potentially not in high-grade gliomas. The subtypes of HGG found in adults, and consequently the subtypes that have been more frequently studied in the context of clinical trials of immunotherapy, are dramatically heterogeneous and often involve copy number variability, without highly-conserved truncal mutations suitable for immune targeting. There are reasons to believe that the juvenile forms of HGG will respond differently to immunotherapy than adult forms of HGG, as they consistently carry a small number of oncogenic driver mutations.

### I. IMMUNOTHERAPIES USING PEPTIDES OF THE DISCLOSURE

A peptide as described herein (e.g., a peptide of one of SEQ ID NOS: 1-107) may be used for immunotherapy in subjects having gliomas. For example, a peptide of one of SEQ ID NOS: 1-107 may be contacted with or used to stimulate a population of T cells to induce proliferation of the T cells that recognize or bind said peptide. A peptide of the disclosure may be administered to a subject, such as a human patient, to enhance the immune response of the subject against a glioma.

A peptide of the disclosure may be included in an active immunotherapy (e.g., a cancer vaccine) or a passive immunotherapy (e.g., an adoptive immunotherapy). Active immunotherapies include immunizing a subject with a purified peptide antigen or an immunodominant peptide (native or modified); alternatively, antigen presenting cells pulsed with a peptide of the disclosure (or transfected with genes encoding an antigen comprising the peptide) may be administered to a subject. The peptide may be modified or contain one or more mutations such as, e.g., a substitution mutation. Passive immunotherapies include adoptive immunotherapies. Adoptive immunotherapies generally involve administering cells to a subject, wherein the cells (e.g., cytotoxic T cells) have been sensitized in vitro to a peptide of the disclosure (scc, e.g., U.S. Pat. No. 7,910,109).

Flow cytometry may be used in the adoptive immunotherapy for rapid isolation of human tumor antigen-specific T-cell clones by using, e.g., T-cell receptor (TCR) Vβ antibodies in combination with carboxyfluorescein succinimidyl ester (CFSE)-based proliferation assay. Scc, e.g., Lcc et al., J. Immunol. Methods, 331:13-26, 2008, which is incorporated by reference for all purposes. Tetramer-guided cell sorting may be used such as, e.g., the methods described in Pollack, et al., J Immunother Cancer. 2014; 2:36, which is herein incorporated by reference for all purposes. Various culture protocols are also known for adoptive immunotherapy and may be used in the methods of the disclosure. Cells may be cultured in conditions which do not require the use of antigen presenting cells (e.g., Hida et al., Cancer Immunol. Immunotherapy, 51:219-228, 2002, which is incorporated by reference). T cells may be expanded under culture conditions that utilize antigen presenting cells, such as dendritic cells (Nestle et al., 1998, incorporated by reference), and artificial antigen presenting cells may be used for this purpose (Maus et al., 2002 incorporated by reference). Additional methods for adoptive immunotherapy are disclosed in Dudley et al. (2003), which is incorporated by reference, that may be used with methods and compositions of the current disclosure. Various methods are known and may be used for cloning and expanding human antigen-specific T cells (see, e.g., Riddell et al., 1990, which is herein incorporated by reference).

The following protocol may be used to generate T cells that selectively recognize peptides of the disclosure. Peptide-specific T-cell lines may be generated from normal donors or HLA-restricted normal donors and patients using methods previously reported (Hida et al., 2002). Briefly, PBMCs (1×10⁵ cells/well) can be stimulated with about 10 μg/ml of each peptide in quadruplicate in a 96-well, U-bottom-microculture plate (Corning Incorporated, Lowell, MA) in about 200 μl of culture medium. The culture medium may consist of 50% AIM-V medium (Invitrogen), 50% RPMI1640 medium (Invitrogen), 10% human AB scrum (Valley Biomedical, Winchester, VA), and 100 IU/ml of interleukin-2 (IL-2). Cells may be restimulated with the corresponding peptide about every 3 days. After 5 stimulations, T cells from each well may be washed and incubated with T2 cells in the presence or absence of the corresponding peptide. After about 18 hours, the production of interferon (IFN)-γ may be determined in the supernatants by ELISA. T cells that secret large amounts of IFN-γ may be further expanded by a rapid expansion protocol (Riddell et al., 1990; Yee et al., 2002b).

An immunotherapy may utilize a peptide of the disclosure that is associated with a cell penetrator, such as a liposome or a cell penetrating peptide (CPP). Antigen presenting cells (such as dendritic cells) pulsed with peptides may be used to enhance antitumour immunity (Celluzzi et al., 1996; Young et al., 1996). Liposomes and CPPs are described in further detail belowAn immunotherapy may utilize a nucleic acid encoding a peptide of the disclosure, wherein the nucleic acid is delivered, e.g., in a viral vector or non-viral vector.

A peptide of the disclosure may be used in an immunotherapy to treat gliomas in a mammalian subject, such as a human patient.

### II. CELL PENETRATING PEPTIDES

A peptide of the disclosure may also be associated with or covalently bound to a cell penetrating peptide (CPP). Cell penetrating peptides that may be covalently bound to a peptide of the disclosure include, e.g., HIV Tat, herpes virus VP22, the Drosophila Antennapedia homeobox gene product, signal sequences, fusion sequences, or protegrin I. Covalently binding a peptide to a CPP can prolong the presentation of a peptide by dendritic cells, thus enhancing antitumour immunity (Wang and Wang, 2002). A peptide of the disclosure (e.g., comprised within a peptide or polyepitope string) may be covalently bound (e.g., via a peptide bond) to a CPP to generate a fusion protein. A peptide or nucleic acid encoding a peptide, according to the current disclosure, may be encapsulated within or associated with a liposome, such as a mulitlamellar, vesicular, or multivesicular liposome.

As used herein, “association” means a physical association, a chemical association or both. For example, an association can involve a covalent bond, a hydrophobic interaction, encapsulation, surface adsorption, or the like.

As used herein, “cell penetrator” refers to a composition or compound which enhances the intracellular delivery of the peptide/polyepitope string to the antigen presenting cell. For example, the cell penetrator may be a lipid which, when associated with the peptide, enhances its capacity to cross the plasma membrane. Alternatively, the cell penetrator may be a peptide. Cell penetrating peptides (CPPs) are known in the art, and include, e.g., the Tat protein of HIV (Frankel and Pabo, 1988), the VP22 protein of HSV (Elliott and O'Hare, 1997) and fibroblast growth factor (Lin et al., 1995).

Cell-penetrating peptides (or “protein transduction domains”) have been identified from the third helix of the Drosophila Antennapedia homeobox gene (Antp), the HIV Tat, and the herpes virus VP22, all of which contain positively charged domains enriched for arginine and lysine residues (Schwarze et al., 2000; Schwarze et al., 1999). Also, hydrophobic peptides derived from signal sequences have been identified as cell-penetrating peptides. (Rojas et al., 1996; Rojas et al., 1998; Du et al., 1998). Coupling these peptides to marker proteins such as β-galactosidase has been shown to confer efficient internalization of the marker protein into cells, and chimeric, in-frame fusion proteins containing these peptides have been used to deliver proteins to a wide spectrum of cell types both in vitro and in vivo (Drin et al., 2002). Fusion of these cell penetrating peptides to a peptide of the disclosure may enhance cellular uptake of the polypeptides.

Cellular uptake may be facilitated by the attachment of a lipid, such as stearate or myristilate, to the polypeptide. Lipidation has been shown to enhance the passage of peptides into cells. The attachment of a lipid moiety is another way that the present invention increases polypeptide uptake by the cell.

A peptide of the disclosure may be included in a liposomal vaccine composition. For example, the liposomal composition may be or comprise a proteoliposomal composition. Methods for producing proteoliposomal compositions that may be used with the present invention are described, e.g., in Neclapu et al. (2007) and Popescu et al. (2007).

By enhancing the uptake of a polypeptide of the disclosure, it may be possible to reduce the amount of protein or peptide required for treatment. This in turn can significantly reduce the cost of treatment and increase the supply of therapeutic agent. Lower dosages can also minimize the potential immunogencity of peptides and limit toxic side effects.

A peptide of the disclosure may be associated with a nanoparticle to form nanoparticle-polypeptide complex. The nanoparticle may be a liposomes or other lipid-based nanoparticle such as a lipid-based vesicle (e.g., a DOTAP: cholesterol vesicle). The nanoparticle may be an iron-oxide based superparamagnetic nanoparticles. Superparamagnetic nanoparticles ranging in diameter from about 10 to 100 nm are small enough to avoid sequestering by the spleen, but large enough to avoid clearance by the liver. Particles this size can penetrate very small capillaries and can be effectively distributed in body tissues. Superparamagnetic nanoparticles-polypeptide complexes can be used as MRI contrast agents to identify and follow those cells that take up the peptide. The nanoparticle may be a semiconductor nanocrystal or a semiconductor quantum dot, both of which can be used in optical imaging. The nanoparticle can be a nanoshell, which comprises a gold layer over a core of silica. One advantage of nanoshells is that polypeptides can be conjugated to the gold layer using standard chemistry. The nanoparticle can be a fullerene or a nanotube (Gupta et al., 2005).

Peptides are rapidly removed from the circulation by the kidney and are sensitive to degradation by proteases in serum. By associating a peptide with a nanoparticle, the nanoparticle-polypeptide complexes of the present invention may protect against degradation and/or reduce clearance by the kidney. This may increase the serum half-life of polypeptides, thereby reducing the polypeptide dose need for effective therapy. Further, this may decrease the costs of treatment, and minimizes immunological problems and toxic reactions of therapy.

### III. POLYEPITOPE STRINGS

A peptide may be included or comprised in a polyepitope string. A polyepitope string is a peptide or polypeptide containing a plurality of antigenic epitopes from one or more antigens linked together. A polyepitope string may be used to induce an immune response in a subject, such as a human subject. Polyepitope strings have been previously used to target malaria and other pathogens (Baraldo et al., 2005; Moorthy et al., 2004; Baird et al., 2004). A polyepitope string may refer to a nucleic acid (e.g., a nucleic acid encoding a plurality of antigens including a peptide of the disclosure) or a peptide or polypeptide (e.g., containing a plurality of antigens including a peptide of the disclosure). A polyepitope string may be included in a cancer vaccine composition.

### IV. APPLICATIONS OF ANTIGENIC PEPTIDES

The disclosure describes the development of and use of antigenic peptides that that are useful for treating and preventing certain gliomas. Antigenic peptides may be produced by chemical synthesis or by molecular expression in a host cell. Peptides can be purified and utilized in a variety of applications including (but not limited to) assays to determine peptide immunogenicity, assays to determine recognition by T cells, peptide vaccines for treatment of gliomas, development of modified TCRs of T cells, and development of antibodies.

Peptides can be synthesized chemically by a number of methods. One common method is to use solid-phase peptide synthesis (SPPS). Generally, SPPS is performed by repeating cycles of alternate N-terminal deprotection and coupling reactions, building peptides from the c-terminus to the n-terminus. The c-terminus of the first amino acid is coupled the resin, wherein then the amine is deprecated and then coupled with the free acid of the second amino acid. This cycle repeats until the peptide is synthesized.

Peptides can also be synthesized utilizing molecular tools and a host cell. Nucleic acid sequences corresponding with antigenic peptides can be synthesized. Synthetic nucleic acids may be synthesized in in vitro synthesizers (e.g., phosphoramidite synthesizer), bacterial recombination system, or other suitable methods. Furthermore, synthesized nucleic acids can be purified and lyophilized, or kept stored in a biological system (e.g., bacteria, yeast). For use in a biological system, synthetic nucleic acid molecules can be inserted into a plasmid vector, or similar. A plasmid vector can also be an expression vector, wherein a suitable promoter and a suitable 3′-polyA tail is combined with the transcript sequence.

The disclosure also describes expression vectors and expression systems that produce antigenic peptides or proteins. These expression systems can incorporate an expression vector to express transcripts and proteins in a suitable expression system. Typical expression systems include bacterial (e.g., E. coli), insect (e.g., SF9), yeast (e.g., S. cerevisiae), animal (e.g., CHO), or human (e.g., HEK 293) cell lines. RNA and/or protein molecules can be purified from these systems using standard biotechnology production procedures.

Assays to determine immunogenicity and/or TCR binding can be performed. One such as is the dextramer flow cytometry assay. Generally, custom-made HLA-matched MHC Class I dextramer:peptide (pMHC) complexes are developed or purchased (Immudex, Copenhagen, Denmark). T cells from peripheral blood mononuclear cells (PBMCs) or tumor-infiltrating lymphocytes (TILs) are incubated the pMHC complexes and stained, which are then run through a flow cytometer to determine if the peptide is capable of binding a TCR of a T cell.

The peptides of the disclosure can also be used to isolate and/or identify T-cell receptors that bind to the peptide. T-cell receptors comprise two different polypeptide chains, termed the T-cell receptor α (TCRα) and β (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T-cell receptor differ from the membrane-bound immunoglobulin that serves as the B-cell receptor: a T-cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Ca domain, where the fold is unlike that of any other immunoglobulin-like domain. The half of the domain that is juxtaposed with the Cβ domain forms a β sheet similar to that found in other immunoglobulin-like domains, but the other half of the domain is formed of loosely packed strands and a short segment of α helix. The intramolecular disulfide bond, which in immunoglobulin-like domains normally joins two β strands, in a Cα domain joins a β strand to this segment of α helix.

There are also differences in the way in which the domains interact. The interface between the V and C domains of both T-cell receptor chains is more extensive than in antibodies, which may make the hinge joint between the domains less flexible. And the interaction between the Cα and Cβ domains is distinctive in being assisted by carbohydrate, with a sugar group from the Cα domain making a number of hydrogen bonds to the Cβ domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the β strand that anchors one end of the loop from one face of the domain to the other. A strand displacement also causes a change in the orientation of the Vβ CDR2 loop in two of the seven Vβ domains whose structures are known. As yet, the crystallographic structures of seven T-cell receptors have been solved to this level of resolution.

The disclosure also describes engineered T cell receptors that bind a peptide of the disclosure, such as a peptide of one of SEQ ID NOS: 1-107. The term “engineered” refers to T cell receptors that have TCR variable regions grafted onto TCR constant regions to make a chimeric polypeptide that binds to peptides and antigens of the disclosure. The TCR may comprise intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

The TCR may comprise non-TCR sequences. Accordingly, also described are TCRs with sequences that are not from a TCR gene. The TCR may be chimeric, in that it contains sequences normally found in a TCR gene, but contains sequences from at least two TCR genes that are not necessarily found together in nature.

### V. ANTIBODIES

The disclosure also describes antibodies that target the peptides of the disclosure, or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, scc, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4:302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (2). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (u), delta (8), gamma (γ), alpha (a), or epsilon (8) chains, respectively. IgG has several subtypes, including, but not limited to, IgG1, IgG2, IgG3, and IgG4. IgM subtypes include IgM1 and IgM2. IgA subtypes include IgA1 and IgA2.

### VI. ANTIBODY CONJUGATES

The disclosure also describes antibodies against a peptide of the disclosure, generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium ¹⁸⁶, rhenium¹⁸⁸, ⁷⁵selenium, ³⁵sulphur, technicium^{99m}) and/or yttrium⁹⁰. ¹²⁵I can be used, and technicium^{99m} and/or indium¹¹¹ can also be used due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl) propionate.

Derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

The antibody may be linked to semiconductor nanocrystals such as those described in U.S. Pat. Nos. 6,048,616; 5,990,479; 5,690,807; 5,505,928; 5,262,357 (all of which are incorporated herein in their entireties); as well as PCT Publication No. 99/26299 (published May 27, 1999). In particular, exemplary materials for use as semiconductor nanocrystals in the biological and chemical assays of the present invention include, but are not limited to those described above, including group II-VI, III-V and group IV semiconductors such as ZnS, ZnSe, ZnTe, CdS, CdSc, CdTe, MgS, MgSe, MgTe, CaS, CaSe, CaTe, SrS, SrSe, SrTe, BaS, BaSc, BaTe, GaN, GaP, GaAs, GaSb, InP, InAs, InSb, AIS, AIP, AlSb, PbS, PbSe, Ge and Si and ternary and quaternary mixtures thereof. Methods for linking semiconductor nanocrystals to antibodies are described in U.S. Pat. Nos. 6,630,307 and 6,274,323.

The present invention concerns immunodetection methods for binding, purifying, removing, quantifying and/or otherwise generally detecting biological components such as T cells or that selectively bind or recognize a peptide of the disclosure. A tetramer assay may be used with the present invention. Tetramer assays generally involve generating soluble peptide-MHC tetramers that may bind antigen specific T lymphocytes, and methods for tetramer assays are described, e.g., in Altman et al. (1996). Some immunodetection methods that may be used include, e.g., enzyme linked immunosorbent assay (ELISA), radioimmunoassay (RIA), immunoradiometric assay, fluoroimmunoassay, chemiluminescent assay, bioluminescent assay, tetramer assay, and Western blot. The steps of various useful immunodetection methods have been described in the scientific literature, such as, e.g., Doolittle and Ben-Zeev, 1999; Gulbis and Galand, 1993; De Jager et al., 1993; and Nakamura et al., 1987, each incorporated herein by reference.

### VII. MHC POLYPEPTIDES

The disclosure describes compositions comprising MHC polypeptides. The MHC polypeptide may comprise at least 2, 3, or 4 MHC polypeptides that may be expressed as separate polypeptides or as a fusion protein. Presentation of antigens to T cells is mediated by two distinct classes of molecules MHC class I (MHC-I) and MHC class II (MHC-II) (also identified as “pMHC” herein), which utilize distinct antigen processing pathways. Peptides derived from intracellular antigens are presented to CD8⁺ T cells by MHC class I molecules, which are expressed on virtually all cells, while extracellular antigen-derived peptides are presented to CD4⁺ T cells by MHC-II molecules. A particular antigen may be identified and presented in the antigen-MHC complex in the context of an appropriate MHC class I or II polypeptide. The genetic makeup of a subject may be assessed to determine which MHC polypeptide is to be used for a particular patient and a particular set of peptides. The MHC class 1 polypeptide may comprise all or part of a HLA-A, HLA-B, HLA-C, HLA-E, HLA-F, HLA-G or CD-1 molecule. The MHC class II polypeptide can comprise all or a part of a HLA-DR, HLA-DQ, or HLA-DP.

Non-classical MHC polypeptides are also contemplated for use in MHC complexes of the invention. Non-classical MHC polypeptides are non-polymorphic, conserved among species, and possess narrow, deep, hydrophobic ligand binding pockets. These binding pockets are capable of presenting glycolipids and phospholipids to Natural Killer T (NKT) cells or certain subsets of CD8+ T-cells such as Qa1, HLA-E-restricted CD8+ T-cells, or MAIT cells. NKT cells represent a unique lymphocyte population that co-express NK cell markers and a semi-invariant T cell receptor (TCR). They are implicated in the regulation of immune responses associated with a broad range of diseases.

### VIII. HOST CELLS

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Transfection can be carried out on any prokaryotic or eukaryotic cell. Electroporation may involve transfection of a human cell. Electroporation may involve transfection of an animal cell. Transfection may involve transfection of a cell line or a hybrid cell type. The cell or cells being transfected may be cancer cells, tumor cells or immortalized cells. Tumor, cancer, immortalized cells or cell lines may be induced and in other instances tumor, cancer, immortalized cells or cell lines enter their respective state or condition naturally. The cells or cell lines can be A549, B-cells, B16, BHK-21, C2C12, C6, CaCo-2, CAP/, CAP-T, CHO, CHO2, CHO-DG44, CHO-KI, COS-1, Cos-7, CV-1, Dendritic cells, DLD-1, Embryonic Stem (ES) Cell or derivative, H1299, HEK, 293, 293T, 293 FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NSO, Panc-1, PC12, PC-3, Peripheral blood cells, Plasma cells, Primary Fibroblasts, RBL, Renca, RLE, SF21, SF9, SH-SY5Y, SK-MES-1, SK—N—SH, SL3, SW403, Stimulus-triggered Acquisition of Pluripotency (STAP) cell or derivate SW403, T-cells, THP-1, Tumor cells, U2OS, U937, peripheral blood lymphocytes, expanded T cells, hematopoietic stem cells, or Vero cells.

### IX. ADDITIONAL AGENTS

### A. Immunostimulators

The method may further comprise administration of an additional agent. The additional agent may be an immunostimulator. The term “immunostimulator” as used herein refers to a compound that can stimulate an immune response in a subject, and may include an adjuvant. An immunostimulator may be an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherichia coli, Salmonella minnesota, Salmonella typhimurium, or Shigella flexneri or specifically with MPL® (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrheac, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

The additional agent may comprise an agonist for pattern recognition receptors (PRR), including, but not limited to Toll-Like Receptors (TLRs), specifically TLRs 2, 3, 4, 5, 7, 8, 9 and/or combinations thereof. Additional agents may comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. The additional agents also may comprise immunostimulatory RNA molecules, such as but not limited to dsRNA, poly I:C or poly I:poly C12U (available as Ampligen®, both poly I:C and poly I:polyC12U being known as TLR3 stimulants), and/or those disclosed in F. Heil et al., “Species-Specific Recognition of Single-Stranded RNA via Toll-like Receptor 7 and 8” Science 303 (5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. An additional agent may be a TLR-4 agonist, such as bacterial lipopolysaccharide (LPS), VSV-G, and/or HMGB-1. Additional agents may comprise TLR-5 agonists, such as flagellin, or portions or derivatives thereof, including but not limited to those disclosed in U.S. Pat. Nos. 6,130,082, 6,585,980, and 7,192,725.

Additional agents may be proinflammatory stimuli released from necrotic cells (e.g., urate crystals). Additional agents may be activated components of the complement cascade (e.g., CD21, CD35, etc.). Additional agents may be activated components of immune complexes. Additional agents also include complement receptor agonists, such as a molecule that binds to CD21 or CD35. The complement receptor agonist may be one that induces endogenous complement opsonization of the synthetic nanocarrier. Immunostimulators may be cytokines, which are small proteins or biological factors (in the range of 5 kD-20 kD) that are released by cells and have specific effects on cell-cell interaction, communication and behavior of other cells. The cytokine receptor agonist may be a small molecule, antibody, fusion protein, or aptamer.

### B. Immunotherapies

The additional therapy may comprise a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

### 1. Inhibition of Co-Stimulatory Molecules

The immunotherapy may comprise an inhibitor of a co-stimulatory molecule. The inhibitor may comprise an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta).

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in antiviral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### 6. Checkpoint Inhibitors and Combination Treatment

The additional therapy limited to, a lipid microsphere, a lipid nanoparticle, immune checkpoint immunotherapy (ICI). These are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. PD-1, PDL1, and PDL2 may be human PD-1, PDL1 and PDL2.

The PD-1 inhibitor may be a molecule that inhibits the binding of PD-1 to its ligand binding partners. The PD-1 ligand binding partners may be PDL1 and/or PDL2. A PDL1 inhibitor may be a molecule that inhibits the binding of PDL1 to its binding partners. PDL1 binding partners may be PD-1 and/or B7-1. The PDL2 inhibitor may be a molecule that inhibits the binding of PDL2 to its binding partners. A PDL2 binding partner may be PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

The PD-1 inhibitor may be an anti-PD-1 antibody (e.g., a human antibody, a humanized antibody, or a chimeric antibody). The anti-PD-1 antibody may be selected from the group consisting of nivolumab, pembrolizumab, and pidilizumab. The PD-1 inhibitor may be an immunoadhesin (e.g., an immunoadhesin comprising an extracellular or PD-1 binding portion of PDL1 or PDL2 fused to a constant region (e.g., an Fc region of an immunoglobulin sequence). The PDL1 inhibitor may comprise AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

The immune checkpoint inhibitor may be a PDL1 inhibitor such as Durvalumab, also known as MEDI4736, atezolizumab, also known as MPDL3280A, avelumab, also known as MSB00010118C, MDX-1105, BMS-936559, or combinations thereof. The immune checkpoint inhibitor may be a PDL2 inhibitor such as rHIgM12B7.

The inhibitor may comprise the heavy and light chain CDRs or VRs of nivolumab, pembrolizumab, or pidilizumab. Accordingly, the inhibitor may comprise the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. The antibody may be one that competes for binding with and/or binds to the same epitope on PD-1, PDL1, or PDL2 as the above-mentioned antibodies. The antibody may have at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. The inhibitor may be one that blocks the CTLA-4 and B7-1 interaction. The inhibitor may be one that blocks the CTLA-4 and B7-2 interaction.

The immune checkpoint inhibitor may be an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

The inhibitor may comprise the heavy and light chain CDRs or VRs of tremelimumab or ipilimumab. Accordingly, the inhibitor may comprise the CDR1, CDR2, and CDR3 domains of the VH region of tremelimumab or ipilimumab, and the CDR1, CDR2 and CDR3 domains of the VL region of tremelimumab or ipilimumab. The antibody may compete for binding with and/or binds to the same epitope on PD-1, B7-1, or B7-2 as the above-mentioned antibodies. The antibody may have at least about 70, 75, 80, 85, 90, 95, 97, or 99% (or any derivable range therein) variable region amino acid sequence identity with the above-mentioned antibodies.

### C. Oncolytic Virus

The additional therapy may comprise an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

### D. Polysaccharides

The additional therapy may comprise polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### E. Neoantigens

The additional therapy may comprise neoantigen administration. Many tumors express mutations. These mutations potentially create new targetable antigens (neoantigens) for use in T cell immunotherapy. The presence of CD8⁺ T cells in cancer lesions, as identified using RNA sequencing data, is higher in tumors with a high mutational burden. The level of transcripts associated with cytolytic activity of natural killer cells and T cells positively correlates with mutational load in many human tumors.

### F. Chemotherapies

The additional therapy may comprise a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidinc analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). Cisplatin may be a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated. The amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. Appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. The chemotherapeutic agent may be administered in an amount effective to cause arrest or regression of the cancer in a host, when the chemotherapy is administered with the construct. The chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### G. Radiotherapy

The additional therapy or prior therapy may comprise radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

The amount of ionizing radiation may be greater than 20 Gy and is administered in one dose. The amount of ionizing radiation may be 18 Gy and is administered in three doses. The amount of ionizing radiation may be at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). The ionizing radiation may be administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

The amount of IR may be presented as a total dose of IR, which is then administered in fractionated doses. For example, the total dose may be 50 Gy administered in 10 fractionated doses of 5 Gy each. The total dose may be 50-90 Gy, administered in 20-60 fractionated doses of 2-3 Gy each. The total dose of IR may be at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). The total dose may be administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. At least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). At least, at most, or exactly 1, 2, 3, 4, 5, 6, 7, 8, 9, 10, 11, or 12 (or any derivable range therein) fractionated doses are administered per day. At least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### H. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### I. Other Agents

It is contemplated that other agents may be used in combination with compositions of the present disclosure to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. Cytostatic or differentiation agents can be used in combination with compositions and methods of the present disclosure to improve the anti-hyperproliferative efficacy of the treatments. Inhibitors of cell adhesion are contemplated to improve the efficacy of the methods and compositions of the disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with compositions and methods of the present disclosure to improve the treatment efficacy.

### X. PROTEINACEOUS COMPOSITIONS

As used herein, a “protein” “peptide” or “polypeptide” refers to a molecule comprising at least five amino acid residues. As used herein, the term “wild-type” refers to the endogenous version of a molecule that occurs naturally in an organism. Wild-type versions of a protein or polypeptide may be employed, however, a modified protein or polypeptide may be employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. A modified/variant protein or polypeptide may have at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. Also described are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The size of a peptide, protein, or polypeptide (wild-type or modified), such as a peptide or protein of the disclosure comprising a peptide of one of SEQ ID NOS: 1-107 may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). It is specifically contemplated that any one or more peptides of SEQ ID NOS: 1-107 may be excluded in the methods or compositions of the disclosure.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous in sequence to at least, or at most 3, 4, 5, 6, 7, 8, or 9 contiguous amino acids of a peptide of one of SEQ ID NOS: 1-107 or nucleic acids encoding a peptide of one of SEQ ID NOS: 1-107. The peptide or polypeptide may be not naturally occurring and/or may be in a combination of peptides or polypeptides.

The polypeptides, peptide, or polynucleotides encoding such polypeptides or peptides may comprise one or more substitutions of the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 (or combinations thereof) of SEQ ID NO:1-107 with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

The protein or polypeptide may comprise amino acids 1 to 2, 3, 4, 5, 6, 7, 8, 9, 10, 11, 12, 13, 14, 15, 16, 17, 18, 19, or 20 (or any derivable range therein) of a peptide of one of SEQ ID NOS: 1-107. The peptides of the disclosure may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) flanking the caboxy and/or flanking the amino end of a peptide comprising or consisting of 3, 4, 5, 6, 7, 8, 9, 10, 11, 12, 13, 14, 15, 16, 17, 18, 19, or 20 contiguous amino acids of a peptide of one of SEQ ID NOS: 1-107.

The protein, polypeptide, or nucleic acid may comprise 1, 2, 3, 44, 5, 6, 7, 8, 9, 10, 11, 12, 13, 14, 15, 16, 17, 18, 19, or 20 (or any derivable range therein) contiguous amino acids of a peptide of one of SEQ ID NOS: 1-107.

The polypeptide, protein, or nucleic acid may comprise at least, at most, or exactly 1, 2, 3, 4, 5, 6, 7, 8, 9, 10, 11, 12, 13, 14, 15, 16, 17, 18, 19, or 20 (or any derivable range therein) contiguous amino acids of a peptide of one of SEQ ID NOS: 1-107 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to a peptide of one of SEQ ID NOS: 1-107.

The disclosure provides for a polypeptide (or a nucleic acid molecule encoding such a polypeptide) starting at position 1, 2, 3, 4, 5, 6, 7, 8, 9, 10, 11, 12, 13, 14, 15, 16, 17, 18, 19, or 20 of a peptide of one of SEQ ID NOS: 1-107 and comprising at least, at most, or exactly 2, 3, 4, 5, 6, 7, 8, 9, 10, 11, 12, 13, 14, 15, 16, 17, 18, or 19 (or any derivable range therein) contiguous amino acids of a peptide of one of SEQ ID NOS: 1-107.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. Areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, the substitution of amino acids whose hydropathy indices which are within ±2 is included. Those that are within ±1 may be included. Those within ±0.5 may be included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. The greatest local average hydrophilicity of a protein, as governed by the hydrophilicity of its adjacent amino acids, may correlate with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, the substitution of amino acids whose hydrophilicity values are within ±2 may be included, or those which are within ±1 may be included, or those within ±0.5 may be included. One may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of a polypeptide with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

Amino acid substitutions may be made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (e.g. conservative amino acid substitutions) may be made in the naturally occurring sequence. Substitutions can be made in that portion of the antibody that lies outside the domain(s) forming intermolecular contacts. Conservative amino acid substitutions can be used that do not substantially change the structural characteristics of the protein or polypeptide (e.g., one or more replacement amino acids that do not disrupt the secondary structure that characterizes the native antibody).

### XI. NUCLEIC ACIDS

Nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

The disclosure provides for polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). The isolated polynucleotide may comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the case of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. One or more particular amino acid residues may be changed using, for example, a site-directed mutagenesis protocol. One or more randomly selected residues may be changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

Nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

The nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing the engineered cells of the disclosure. The nucleic acid molecules may be oligonucleotides.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### XII. POLYPEPTIDE EXPRESSION

The disclosure also provides for nucleic acid molecule encoding polypeptides or peptides of the disclosure (e.g antibodies, TCR genes, MHC molecules, and immunogenic peptides). These may be generated by methods known in the art, e.g., isolated from B cells of mice that have been immunized and isolated, phage display, expressed in any suitable recombinant expression system and allowed to assemble to form antibody molecules or by recombinant methods.

The nucleic acid molecules may be used to express large quantities of polypeptides. If the nucleic acid molecules are derived from a non-human, non-transgenic animal, the nucleic acid molecules may be used for humanization of the antibody or TCR genes.

### A. Vectors

Also contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. Expression vectors comprising nucleic acid molecules may encode fusion proteins, antigenic peptides and polypeptides, TCR genes, MHC molecules, modified antibodies, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. A vector that encodes a functionally complete human CH or CL immunoglobulin sequence with appropriate restriction sites engineered so that any VH or VL sequence can be easily inserted and expressed. A vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed for use in the methods of the disclosure to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau ct al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### D. Host Cells

Contemplated are the use of host cells into which a recombinant expression vector has been introduced. Polypeptides can be expressed in a variety of cell types. An expression construct encoding a polypeptide or peptide of the disclosure can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### XIII. FORMULATIONS AND CULTURE OF THE CELLS

The cells of the disclosure may be specifically formulated and/or they may be cultured in a particular medium. The cells may be formulated in such a manner as to be suitable for delivery to a recipient without deleterious effects.

The medium can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the perspective of preventing contamination with heterogeneous animal-derived components, serum can be derived from the same animal as that of the stem cell(s). The serum-free medium refers to medium with no unprocessed or unpurified scrum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptocthanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

The medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescinc 2HCl; Sodium Selenite; and/or T3 (triodo-I-thyronine). One or more of these may be explicitly excluded.

The medium may further comprise vitamins. The medium may comprise 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. The medium may comprise or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. The vitamins may include or consist essentially of biotin, DL alpha tocopherol acetate, DL alpha-tocopherol, vitamin A, or combinations or salts thereof. The medium may further comprise proteins. The proteins may comprise albumin or bovine serum albumin, a fraction of BSA, catalase, insulin, transferrin, superoxide dismutase, or combinations thereof. The medium may further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. The medium may comprise one or more of the following: a B-27® supplement, xeno-free B-27® supplement, GS21TM supplement, or combinations thereof. The medium may comprise or futher comprises amino acids, monosaccharides, inorganic ions. The amino acids may comprise arginine, cystine, isoleucine, leucine, lysine, methionine, glutamine, phenylalanine, threonine, tryptophan, histidine, tyrosine, or valine, or combinations thereof. The inorganic ions may comprise sodium, potassium, calcium, magnesium, nitrogen, or phosphorus, or combinations or salts thereof. The medium may further comprise one or more of the following: molybdenum, vanadium, iron, zinc, selenium, copper, or manganese, or combinations thereof. The medium may comprise or consist essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. One or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. One or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

The cells of the disclosure may be specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. The cells may be in a formulated cell suspension that is stable at room temperature for 1, 2, 3, or 4 hours or more from time of thawing.

The method may further comprise priming the T cells. The T cells may be primed with antigen presenting cells. The antigen presenting cells may present tumor antigens or peptides, such as those disclosed herein.

The cells of the disclosure may comprise an exogenous TCR, which may be of a defined antigen specificity, such as defined antigen specificity to one of SEQ ID NOS: 1-107. The TCR can be selected based on absent or reduced alloreactivity to the intended recipient (examples include certain virus-specific TCRs, xeno-specific TCRs, or cancer-testis antigen-specific TCRs). In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. The choice of exogenous TCR may not necessarily be defined based on lack of alloreactivity. The endogenous TCR genes may have been modified by genome editing so that they do not express a protein. Methods of gene editing such as methods using the CRISPR/Cas9 system are known in the art and described herein.

### XIV. ADMINISTRATION OF THERAPEUTIC COMPOSITIONS

Methods of the disclosure relate to the treatment of subjects with cancer. The treatment may be directed to those that have or have been determined to have a cancer for a particular peptide of the disclosure, such as a peptide of one of SEQ ID NOS: 1-107. The methods may be employed with respect to individuals who have tested positive for such cancer, who have one or more symptoms of a cancer, or who are deemed to be at risk for developing such a cancer.

The therapy provided herein may comprise administration of a combination of therapeutic agents, such as a first anti-cancer therapy and a second anti-cancer therapy. The therapies may be administered in any suitable manner known in the art. For example, the first and second cancer treatment may be administered sequentially (at different times) or concurrently (at the same time). The first and second cancer treatments may be administered in a separate composition. The first and second cancer treatments may be in the same composition.

Described herein are compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. The cancer therapy may be administered intravenously, intramuscularly, subcutaneously, topically, orally, transdermally, intraperitoneally, intraorbitally, by implantation, by inhalation, intrathecally, intraventricularly, or intranasally. The antibiotic may be administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. A unit dose may comprise a single administrable dose.

The quantity to be administered, both according to number of treatments and unit dose, depends on the treatment effect desired. An effective dose is understood to refer to an amount necessary to achieve a particular effect. Doses in the range from 10 mg/kg to 200 mg/kg may affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

The effective dose of the pharmaceutical composition may be one which can provide a blood level of about 1 μM to 150 μM. The effective dose may be one that provides a blood level of about 4 μM to 10 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 5 μM; or about 25 μM to 150 μM; or about 25 μM to 100 μM; or about 25 μM to 50 μM; or about 50 μM to 150 μM; or about 50 μM to 100 μM (or any range derivable therein). The dose may be one that can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. The therapeutic agent may be one that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

A peptide of the disclosure may be comprised in a vaccine composition and administered to a subject to induce a therapeutic immune response in the subject towards a cancer. A vaccine composition for pharmaceutical use in a subject may comprise a peptide composition disclosed herein and a pharmaceutically acceptable carrier.

The phrases “pharmaceutical,” “pharmaceutically acceptable,” or “pharmacologically acceptable” refers to molecular entities and compositions that do not produce an adverse, allergic or other untoward reaction when administered to an animal, such as, for example, a human, as appropriate. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington: The Science and Practice of Pharmacy, 21st edition, Pharmaceutical Press, 2011, incorporated herein by reference). Except insofar as any conventional carrier is incompatible with the active ingredient, its use in the vaccine compositions of the present invention is contemplated.

As used herein, a “protective immune response” refers to a response by the immune system of a mammalian host to a cancer. A protective immune response may provide a therapeutic effect for the treatment of a cancer, e.g., decreasing tumor size, increasing survival, etc.

The vaccine composition may be administered by microstructured transdermal or ballistic particulate delivery. Microstructures as carriers for vaccine formulation are a desirable configuration for vaccine applications and are widely known in the art (Gerstel and Place 1976 (U.S. Pat. No. 3,964,482); Ganderton and McAinsh 1974 (U.S. Pat. No. 3,814,097); U.S. Pat. Nos. 5,797,898, 5,770,219 and 5,783,208, and U.S. Patent Application 2005/0065463). Such a vaccine composition formulated for ballistic particulate delivery may comprise an isolated peptide disclosed herein immobilized on a surface of a support substrate. A support substrate can include, but is not limited to, a microcapsule, a microparticle, a microsphere, a nanocapsule, a nanoparticle, a nanosphere, or a combination thereof.

A vaccine composition may comprise an immobilized or encapsulated peptide or antibody as disclosed herein and a support substrate. A support substrate can include, but is not limited to, a lipid microsphere, a lipid nanoparticle, an ethosome, a liposome, a niosome, a phospholipid, a sphingosome, a surfactant, a transferosome, an emulsion, or a combination thereof. The formation and use of liposomes and other lipid nano- and microcarrier formulations is generally known to those of ordinary skill in the art, and the use of liposomes, microparticles, nanocapsules and the like have gained widespread use in delivery of therapeutics (e.g., U.S. Pat. No. 5,741,516, specifically incorporated herein in its entirety by reference). Numerous methods of liposome and liposome-like preparations as potential drug carriers, including encapsulation of peptides, have been reviewed (U.S. Pat. Nos. 5,567,434; 5,552,157; 5,565,213; 5,738,868 and 5,795,587, each of which is specifically incorporated in its entirety by reference).

In addition to the methods of delivery described herein, a number of alternative techniques are also contemplated for administering the disclosed vaccine compositions. By way of nonlimiting example, a vaccine composition may be administered by sonophoresis (i.e., ultrasound) which has been used and described in U.S. Pat. No. 5,656,016 for enhancing the rate and efficacy of drug permeation into and through the circulatory system; intraosscous injection (U.S. Pat. No. 5,779,708), or feedback-controlled delivery (U.S. Pat. No. 5,697,899), and each of the patents in this paragraph is specifically incorporated herein in its entirety by reference.

### XV. DETECTION AND VACCINATION KITS

A peptide or antibody of the disclosure may be included in a kit. The peptide or antibody in the kit may be detectably labeled or immobilized on a surface of a support substrate also comprised in the kit. The peptide(s) or antibody may, for example, be provided in the kit in a suitable form, such as sterile, lyophilized, or both.

The support substrate comprised in a kit of the invention may be selected based on the method to be performed. By way of nonlimiting example, a support substrate may be a multi-well plate or microplate, a membrane, a filter, a paper, an emulsion, a bead, a microbead, a microsphere, a nanobead, a nanosphere, a nanoparticle, an ethosome, a liposome, a niosome, a transferosome, a dipstick, a card, a celluloid strip, a glass slide, a microslide, a biosensor, a lateral flow apparatus, a microchip, a comb, a silica particle, a magnetic particle, or a self-assembling monolayer.

As appropriate to the method being performed, a kit may further comprise one or more apparatuses for delivery of a composition to a subject or for otherwise handling a composition of the invention. By way of nonlimiting example, a kit may include an apparatus that is a syringe, an eye dropper, a ballistic particle applicator (e.g., applicators disclosed in U.S. Pat. Nos. 5,797,898, 5,770,219 and 5,783,208, and U.S. Patent Application 2005/0065463), a scoopula, a microslide cover, a test strip holder or cover, and such like.

A detection reagent for labeling a component of the kit may optionally be comprised in a kit for performing a method of the present invention. The labeling or detection reagent may be selected from a group comprising reagents used commonly in the art and including, without limitation, radioactive elements, enzymes, molecules which absorb light in the UV range, and fluorophores such as fluorescein, rhodamine, auramine, Texas Red, AMCA blue and Lucifer Yellow. A kit may be provided comprising one or more container means and a BST protein agent already labeled with a detection reagent selected from a group comprising a radioactive element, an enzyme, a molecule which absorbs light in the UV range, and a fluorophore.

When reagents and/or components comprising a kit are provided in a lyophilized form (lyophilisate) or as a dry powder, the lyophilisate or powder can be reconstituted by the addition of a suitable solvent. The solvent may be a sterile, pharmaceutically acceptable buffer and/or other diluent. It is envisioned that such a solvent may also be provided as part of a kit.

When the components of a kit are provided in one and/or more liquid solutions, the liquid solution may be, by way of non-limiting example, a sterile, aqueous solution. The compositions may also be formulated into an administrative composition. In this case, the container means may itself be a syringe, pipette, topical applicator or the like, from which the formulation may be applied to an affected area of the body, injected into a subject, and/or applied to or mixed with the other components of the kit.

### XVI. SEQUENCES

TABLE 1**

| | | SEQ | NetMHC | | HLAthena | | |
|---|---|---|---|---|---|---|---|
| | | ID | BA 4.1 | NetMHC | (log | PRIME | |
| HLA allele | Peptide** | NO: | (IC50)* | EL 4.1† | likelihood)† | score | Gene |
| HLA-A*02:01 | tLSQAIVKv | 1 | 48.4 | 0.9274 | 0.9988 | 0.1998 | U2SURP |
| | SLIGYslFSV | 2 | 3.37 | 0.6972 | 0.9965 | 0.1858 | SLC37A3 |
| | SLINPTviV | 3 | 60.73 | 0.7517 | 0.9776 | 0.1839 | TPRKB |
| | KLTAqvEEL | 4 | 65.12 | 0.8358 | 0.9320 | 0.1837 | NUMA1 |
| | FLaqKCHTL | 5 | 10.71 | 0.9180 | 0.8958 | 0.1991 | HAUS2 |
| | LLGNFeeSV | 6 | 26.43 | 0.5995 | 0.8932 | 0.1681 | FAM214A |
| | YLEHYLdnL | 7 | 45.25 | 0.4447 | 0.6053 | 0.1900 | ING4 |
| HLA-A*02:02 | tLSQAIVKv | 1 | 16.62 | 0.9436 | 0.9948 | 0.1727 | U2SURP |
| | FLaqKCHTL | 5 | 3.64 | 0.9687 | 0.9995 | 0.1815 | HAUS2 |
| | KLTAqvEEL | 4 | 11.43 | 0.9476 | 0.9852 | 0.1580 | NUMA1 |
| | LLGNFeeSV | 6 | 14.71 | 0.6782 | 0.9560 | 0.1650 | FAM214A |
| | SLIGYslFSV | 2 | 3.93 | 0.6058 | 0.9363 | 0.1952 | SLC37A3 |
| | YLEHYLdnL | 7 | 10.84 | 0.6594 | 0.8696 | 0.1965 | ING4 |
| HLA-A*02:03 | tLSQAIVKv | 1 | 11.65 | 0.9395 | 0.9991 | 0.1999 | U2SURP |
| | SLIGYslFSV | 2 | 3.21 | 0.6362 | 0.9985 | 0.1922 | SLC37A3 |
| | SLINPTviV | 3 | 17.65 | 0.7997 | 0.9189 | 0.1858 | TPRKB |
| | FLaqKCHTL | 5 | 5.19 | 0.9181 | 0.7860 | 0.1964 | HAUS2 |
| | KLTAqvEEL | 4 | 80.12 | 0.6369 | 0.7784 | 0.1624 | NUMA1 |
| HLA-A*02:06 | tLSQAIVKv | 1 | 102.87 | 0.8465 | 0.9357 | 0.1800 | U2SURP |
| | KLTAqvEEL | 4 | 262.01 | 0.5919 | 0.7728 | 0.1760 | NUMA1 |
| | FLaqKCHTL | 5 | 32.56 | 0.7320 | 0.7247 | 0.1773 | HAUS2 |
| | IqaGIFQEF | 8 | 121.12 | 0.5148 | 0.6038 | 0.1706 | RAD1 |
| | SLINPTviV | 3 | 83.9 | 0.6583 | 0.5676 | 0.1695 | TPRKB |
| HLA-A*03:01 | KSAPSTGVVK | 9 | 110.13 | 0.7739 | 0.7688 | 0.1883 | H3-3A |
| | STwaRSWAY | 10 | 172.39 | 0.3368 | 0.5450 | 0.1685 | EPSTI1 |
| | KSAPSTGRVK | 11 | 128.42 | 0.6381 | 0.3947 | 0.1758 | H3-3A |
| HLA-A*11:01 | KSAPSTGVVK | 9 | 63.21 | 0.7453 | 0.9082 | 0.1875 | H3-3A |
| | STwaRSWAYR | 10 | 16.05 | 0.3733 | 0.7668 | 0.1800 | EPSTI1 |
| | STwaRSWAY | 10 | 25.24 | 0.6095 | 0.6110 | 0.1658 | EPSTI1 |
| | KSAPSTGRVK | 11 | 163.83 | 0.4444 | 0.6089 | 0.1758 | H3-3A |
| HLA-A*23:01 | VYKdrLIYF | 12 | 33.83 | 0.9696 | 0.9992 | 0.2102 | KLHDC1 |
| | VYLSAqvQL | 13 | 75.81 | 0.9129 | 0.9971 | 0.1919 | RNF217 |
| | SYFEkeTLTF | 14 | 7.28 | 0.9830 | 0.9925 | 0.1821 | ZNF431 |
| | TYNWKglLF | 15 | 22.62 | 0.9039 | 0.9894 | 0.2079 | ITGA7 |
| | PWFEglESRF | 16 | 89.32 | 0.5657 | 0.9882 | 0.1601 | DFFB |
| | RApeTWFEF | 17 | 40.99 | 0.5297 | 0.9138 | 0.1895 | PLA2G4C |
| | IqaGIFQEF | 8 | 70.42 | 0.6968 | 0.8729 | 0.1760 | RAD1 |
| | YFEkeTLTF | 18 | 77.57 | 0.8138 | 0.8658 | 0.1887 | ZNF431 |
| | MYGSLDsaF | 19 | 36.93 | 0.6133 | 0.5794 | 0.1661 | INTU |
| HLA-A*24:02 | VYKdrLIYF | 12 | 45.27 | 0.9756 | 0.9997 | 0.2102 | KLHDC1 |
| | SYFEkeTLTF | 14 | 10.96 | 0.9802 | 0.9958 | 0.1833 | ZNF431 |
| | TYNWKglLF | 15 | 20.3 | 0.9509 | 0.9933 | 0.2111 | ITGA7 |
| | VYLSAqvQL | 13 | 105.38 | 0.3454 | 0.9932 | 0.1868 | RNF217 |
| | PWFEglESRF | 16 | 229.12 | 0.5153 | 0.9885 | 0.1620 | DFFB |
| | GYmdYAQARF | 20 | 44.95 | 0.8115 | 0.9464 | 0.1827 | CHKA |
| | RApeTWFEF | 17 | 47.21 | 0.6137 | 0.8937 | 0.1807 | PLA2G4C |
| | IqaGIFQEF | 8 | 119.42 | 0.6824 | 0.7917 | 0.1824 | RAD1 |
| | YFEkeTLTF | 18 | 94.36 | 0.8493 | 0.7021 | 0.1853 | ZNF431 |
| | RYQTMkgLI | 21 | 73.71 | 0.3262 | 0.6823 | 0.1895 | POLM |
| | MYGSLDsaF | 19 | 33.61 | 0.7527 | 0.5108 | 0.1763 | INTU |
| HLA-A*30:01 | KSAPSTGRVK | 11 | 55.04 | 0.6001 | 0.9594 | 0.1683 | H3-3A |
| | KSAPSTGVVK | 9 | 62.08 | 0.6259 | 0.9082 | 0.1667 | H3-3A |
| | RIktLRRCY | 22 | 112.88 | 0.3484 | 0.9422 | 0.1961 | MSANTD2 |
| | RlrSKRENV | 23 | 43.02 | 0.3723 | 0.8896 | 0.1748 | BIRC6 |
| | KTRLqaRPR | 24 | 21.52 | 0.5041 | 0.7513 | 0.1648 | AGBL5 |
| HLA-A*31:01 | KTRLqaRPR | 24 | 12.03 | 0.7528 | 0.8919 | 0.1794 | AGBL5 |
| | GYmdYAQAR | 25 | 85.81 | 0.4744 | 0.8553 | 0.1843 | CHKA |
| | FLFtsRIRR | 26 | 22.59 | 0.6695 | 0.7256 | 0.1703 | COX20 |
| | CIKTRLqaR | 27 | 35.6 | 0.4492 | 0.7012 | 0.1859 | AGBL5 |
| | HFLFtsRIR | 28 | 52.04 | 0.4472 | 0.6404 | 0.1635 | COX20 |
| | YTNRlrSKR | 29 | 15.53 | 0.7924 | 0.3820 | 0.1679 | BIRC6 |
| HLA-A*32:01 | RApeTWFEF | 17 | 307.65 | 0.3090 | 0.4626 | 0.1865 | PLA2G4C |
| | IqaGIFQEF | 8 | 228.74 | 0.5725 | 0.2651 | 0.1713 | RAD1 |
| HLA-A*33:01 | STwaRSWAYR | 10 | 8.46 | 0.6521 | 0.9388 | 0.2051 | EPSTI1 |
| | CIKTRLqaR | 27 | 85.01 | 0.4301 | 0.8160 | 0.1679 | AGBL5 |
| | GYmdYAQAR | 25 | 261.9 | 0.3800 | 0.7282 | 0.1573 | CHKA |
| | YTNRlrSKR | 29 | 36.11 | 0.6707 | 0.5681 | 0.1749 | BIRC6 |
| | HFLFtsRIR | 28 | 59.19 | 0.5012 | 0.5311 | 0.1827 | COX20 |
| HLA-A*33:03 | STwaRSWAYR | 10 | 5.85 | 0.7785 | 0.9366 | 0.1749 | EPSTI1 |
| | TWArsWAYR | 30 | 19.7 | 0.5386 | 0.8544 | 0.1679 | EPSTI1 |
| | CIKTRLqaR | 27 | 51.61 | 0.4813 | 0.7582 | 0.2051 | AGBL5 |
| | HFLFtsRIR | 28 | 45.18 | 0.5354 | 0.7464 | 0.1755 | COX20 |
| | FLFtsRIRR | 26 | 15.83 | 0.7663 | 0.6599 | 0.1791 | COX20 |
| | YTNRlrSKR | 29 | 14.95 | 0.8109 | 0.6334 | 0.1827 | BIRC6 |
| HLA-A*68:01 | STwaRSWAYR | 10 | 6.03 | 0.7090 | 0.6348 | 0.1849 | EPSTI3 |
| | YTNRlrSKR | 29 | 13.41 | 0.8234 | 0.5234 | 0.1633 | BIRC6 |
| | FLFtsRIRR | 26 | 16.86 | 0.6341 | 0.4324 | 0.1703 | COX20 |
| HLA-A*68:02 | tLSQAIVKv | 1 | 497.55 | 0.4229 | 0.8688 | 0.1859 | U2SURP |
| HLA-B*08:01 | FLaqKCHTL | 5 | 16.49 | 0.9173 | 0.9856 | 0.1936 | HAUS2 |
| | YQTMkgLIL | 31 | 505.77 | 0.3047 | 0.6491 | 0.1786 | POLM |
| HLA-B*15:01 | IqaGIFQEF | 8 | 12.08 | 0.9699 | 0.9983 | 0.1977 | RAD1 |
| HLA-B*58:03 | RApeTWFEF | 17 | 198.83 | 0.4831 | 0.6047 | 0.1845 | PLA2G4C |

**alternative splice junction is represented by lower case

### XVII. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Targeting Alternative Splicing Variants to Stimulate Anti-Tumor Immunity in a Novel Syngeneic Mouse Model of H3G34R High-Grade Glioma

Juvenile presentation of HGG has recently been shown to frequently involve somatic mutations of the H3-3A gene, which encodes the histone H3 variants. Missense mutations at codons K27 or G34 have divergent effects on gene expression that is context-dependent. H3G34R/V HGG display distinct characteristics that differentiate it from the better-characterized H3K27M HGG. H3G34R/V HGG most commonly occur later in childhood, and is typically cortical and lobar in location, in contrast with the midline or diencephalic location of H3K27M HGG. Concurrent TP53, ATRX/DAXX, and PDGFRA alterations are frequent in H3G34R/V HGGs. Profiling of resected H3G34R/V tumors has revealed that they are relatively devoid of infiltrating immune cells, indicating that there may be 1) few immunogenic tumor-specific antigens (TSAs), 2) inhibited trafficking of cytotoxic T lymphocytes (CTLs) into the tumor microenvironment, and 3) immune-evasive characteristics of HGG within the tumor microenvironment.

To address the paucity of identified TSAs in H3G34R/V HGG, the inventors identified peptide sequences that result from alternate exon joining events highly enriched in cancer cells that are predicted to bind the HLA class I alleles carried by the individual patient. In the present study, the inventors identified three candidate peptides from the transcriptome of an H3G34R HGG patient, which were also predicted to bind the MHC I H-2K^{b} allele carried by C57Bl/6J mice. Using the Replication-Competent Avian leukosis virus long terminal repeat with Splice acceptor (RCAS)/tumor virus-a (tv-a) system they generated syngeneic H3G34R tumor lines, which allowed them to test the efficacy of targeted dendritic cell (DC) vaccination in combination with anti-PD-1 checkpoint blockade in an immunocompetent pre-clinical model.

### a. Results

### 1. Generation of Syngeneic Murine H3G34R Gliomas

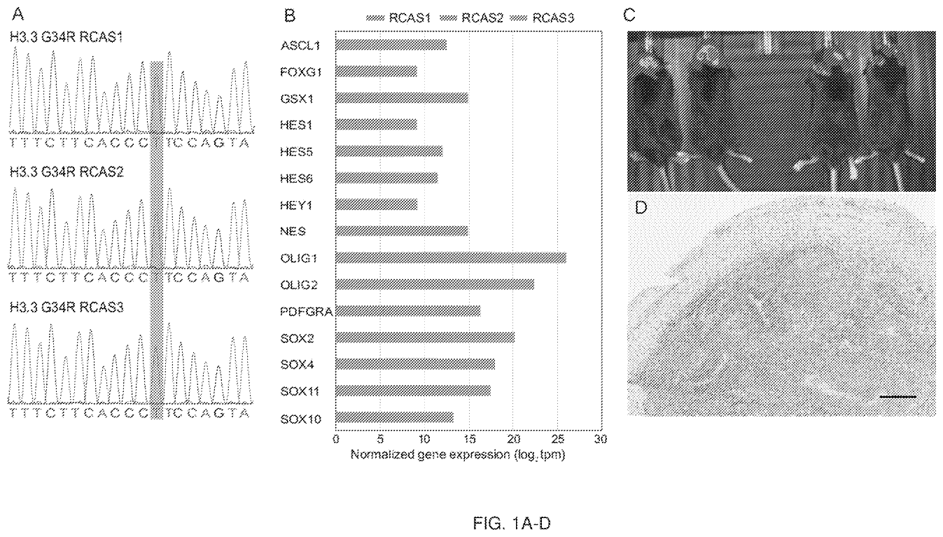

The inventors used the RCAS/tv-A system to develop a preclinical model of H3G34R HGG. Intraventricular injections of Ova cells producing viruses encoding PDGF-B, Cre recombinase, and H3-3A G34R into postnatal day 2-3 C56Bl/6J Tp53^{fl/fl} Tg (Nestin-tv-A) mice were made, and three non-clonal tumor lines were established from three different animals. The presence of the H3G34R mutation in each line was confirmed by genomic PCR (FIG. 1A). From RNA sequencing, they found that the three populations express similar levels of nestin (Nes) and PDGF-α receptor transcripts (Pdgfra), and mRNAs specifying transcription factor (TF) genes that maintain multipotent neural progenitors (FoxG1, Hes1, Hes5, Hey1, Sox2, and Olig2), as well as regulating neuronal (Ascl1, and Gsx1, Hes6, and Sox4), and oligodendrocyte lineages (Olig1, Olig2, and Sox10) of the ventral telencephalon (FIG. 1B). Orthotopic delivery of 1×10⁵ H3G34R/RCAS2 cells, expressing firefly luciferase, resulted in the robust growth of intracranial tumors after 28 days (FIGS. 1C and D).

2. Treatment with Tumor Lysate-Pulsed Dendritic Cells and Anti-PD-1 Monoclonal Antibodies

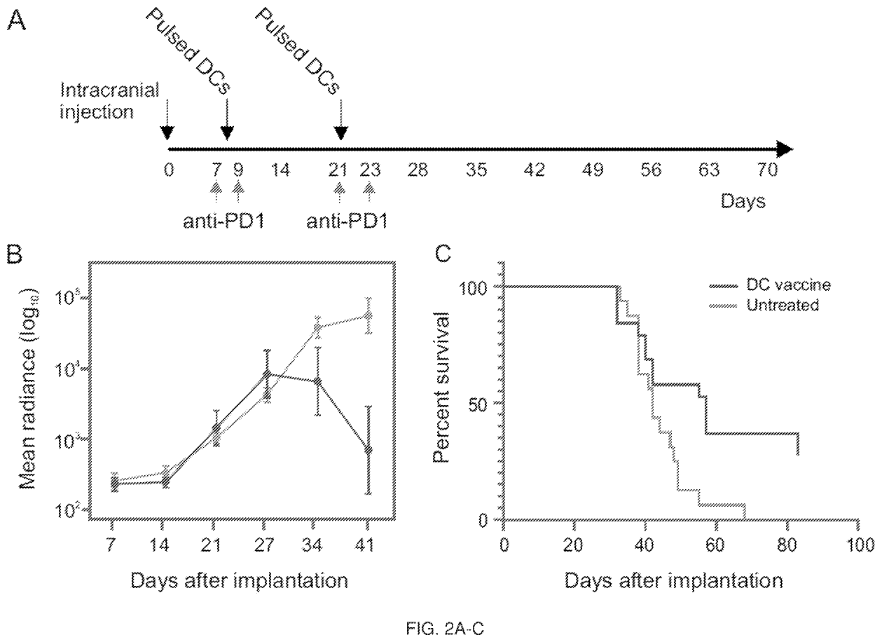

The inventors next asked whether DC vaccination together with anti-PD-1 checkpoint blockade would provide a survival benefit in this preclinical model. H3G34R/RCAS2 tumor cells were implanted into 20 animals. At day 7 post-implantation, 10⁶ spleen derived DCs pulsed with tumor lysate were injected intradermally, and PD-1 monoclonal antibody (mAb) was injected intraperitoneally into ten animals comprising the treatment group. Control animals were injected with PD-1 mAb and a nonspecific isotype control, but did not receive DC vaccination. PD-1 mAb was again administered 48 hours later. The treatment group received a second round of DC vaccination and PD-1 mAb after 14 days (FIG. 2A); tumor growth was monitored by chemiluminescence imaging. Tumors grew at approximately the same rate in both treated and untreated animals up until 28 days post implantation, at which point the mean radiance in the treatment group plateaued and subsequently declined (FIG. 2B), concordant with the survival data (FIG. 2C).

3. Identification of Candidate Neoantigens from an H3G34R Tumor

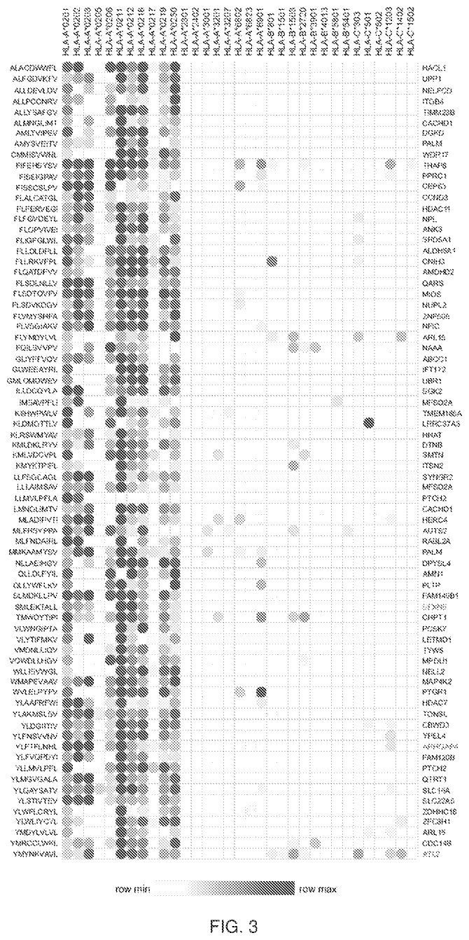

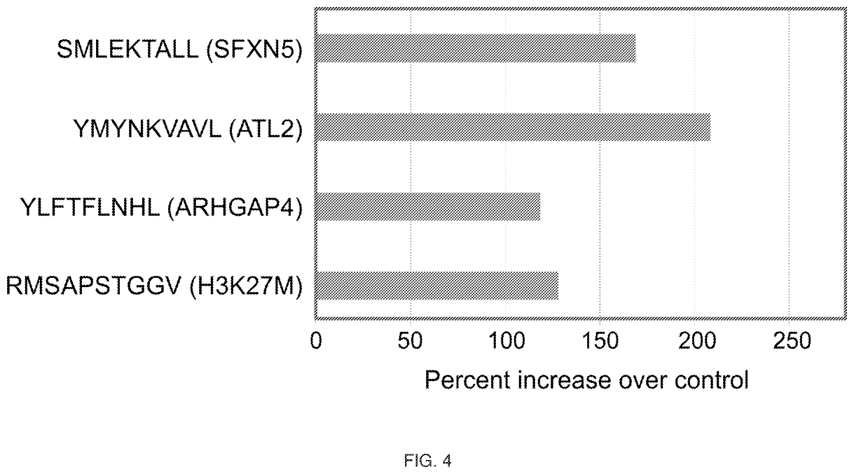

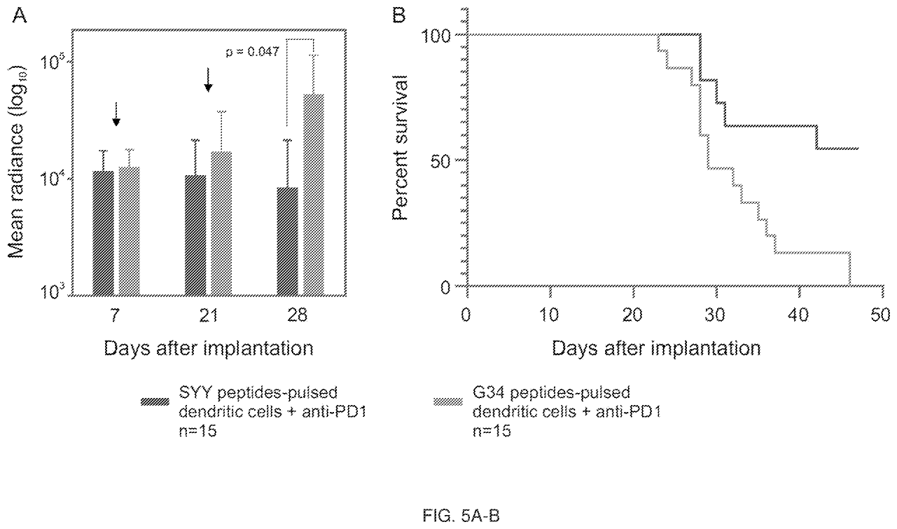

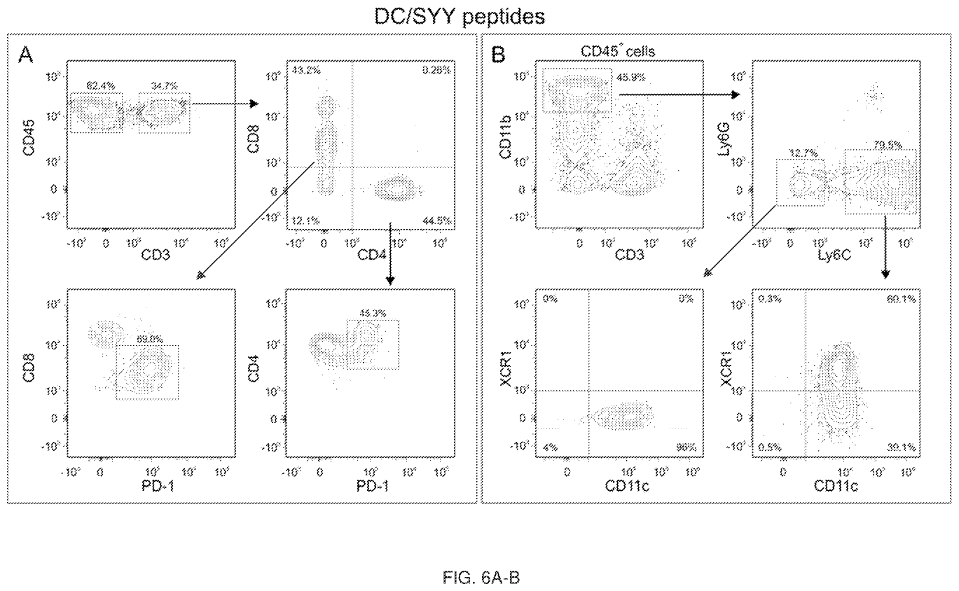

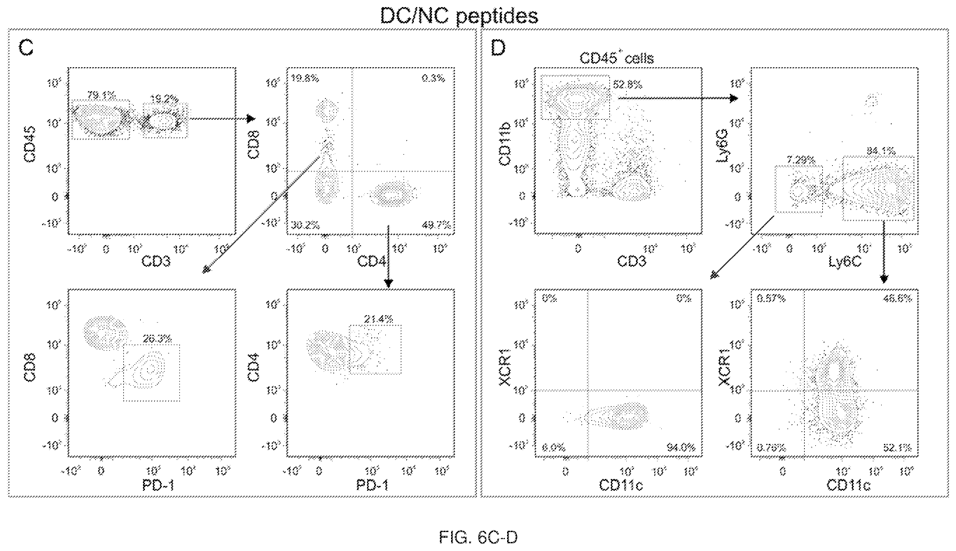

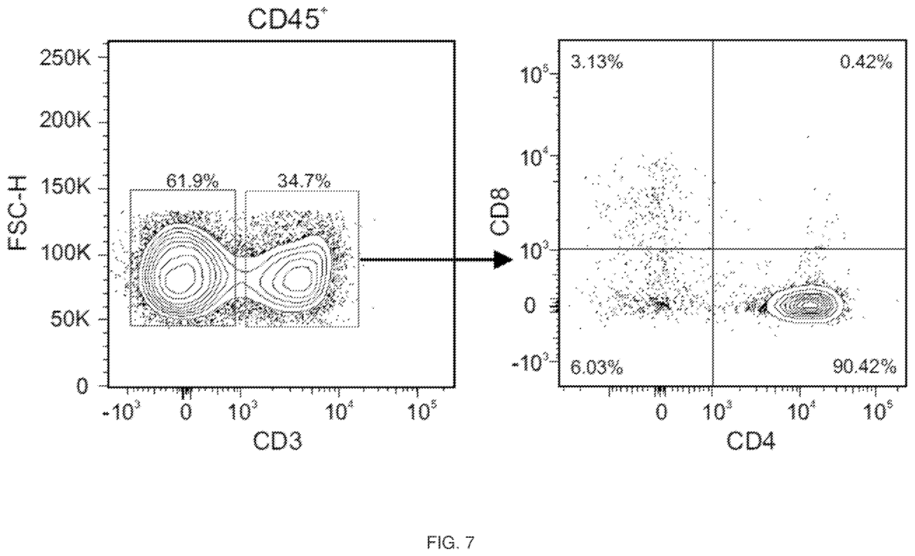

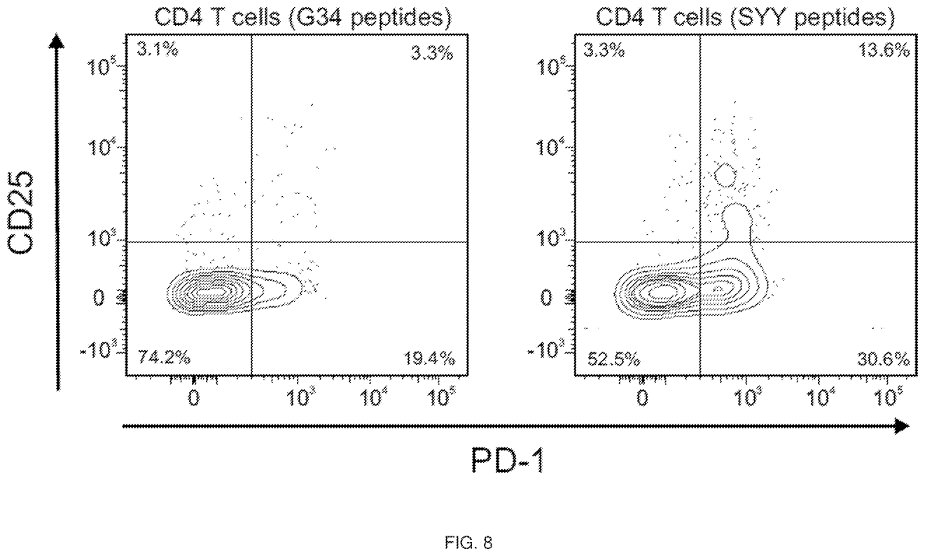

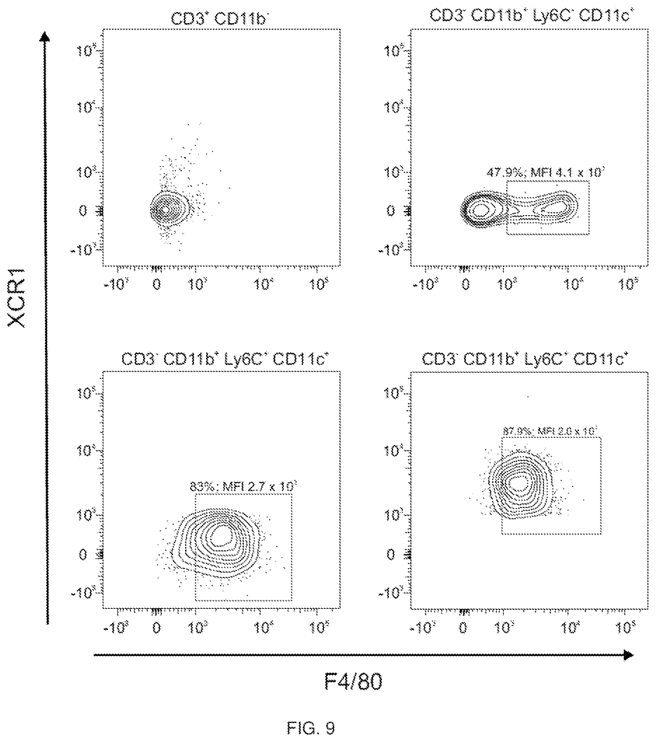

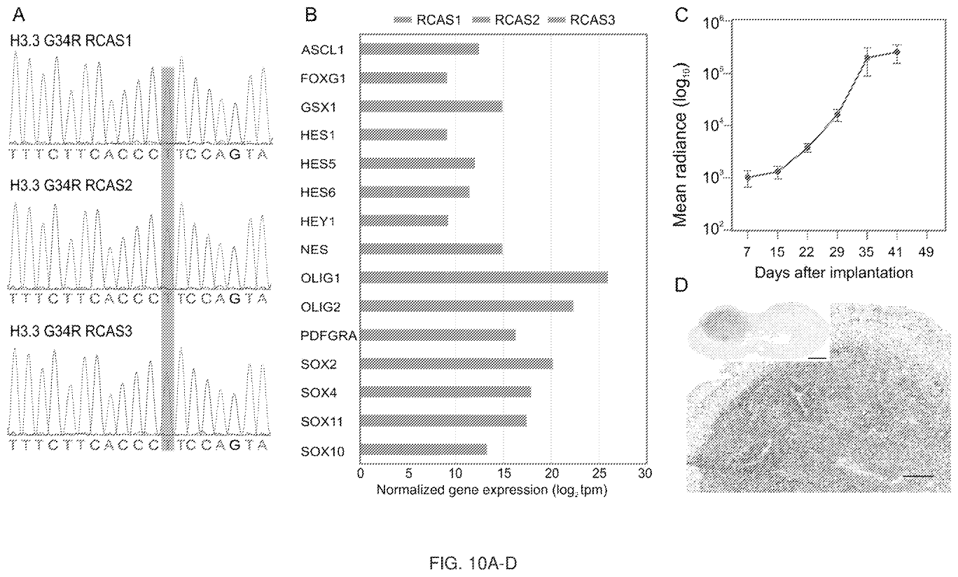

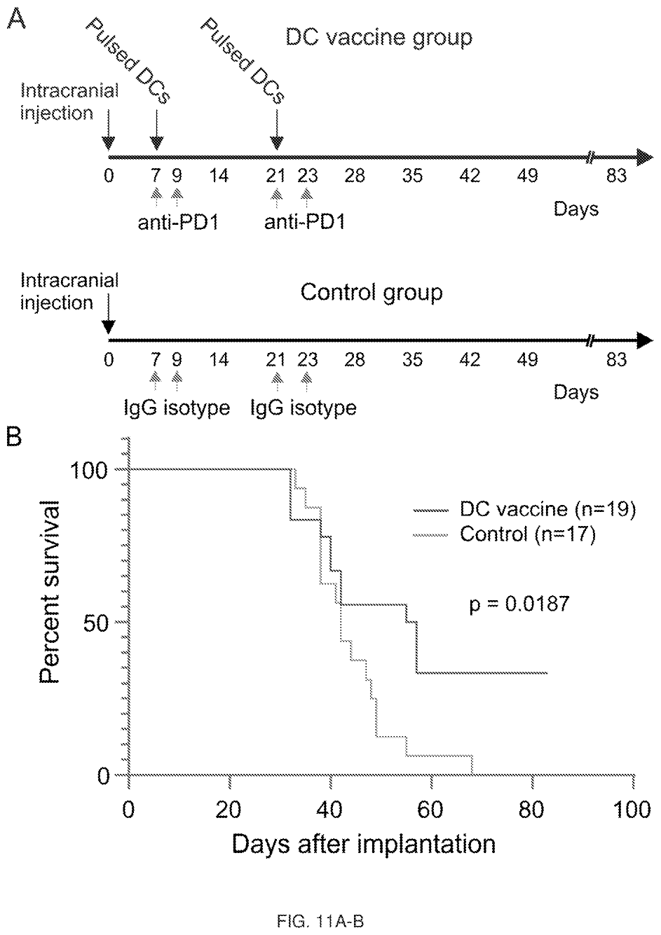

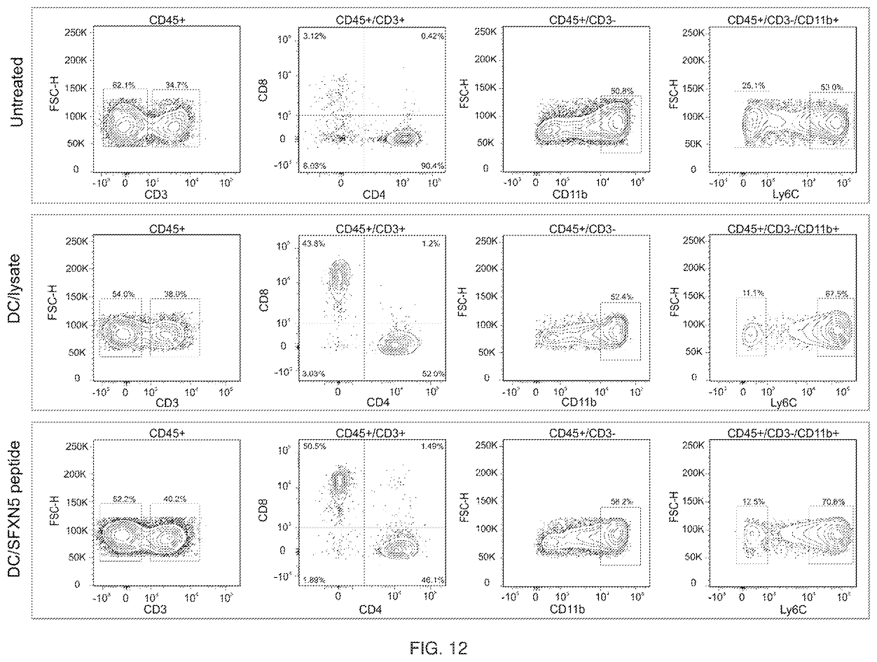

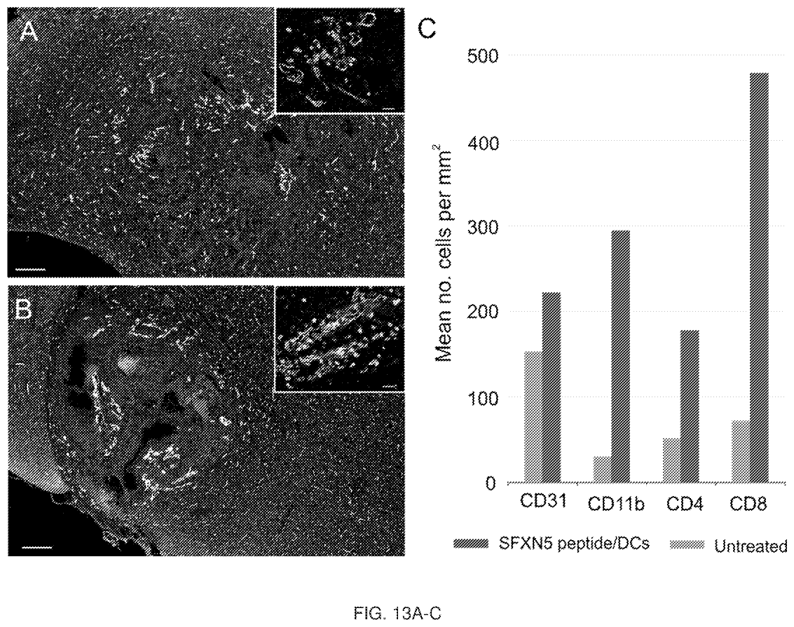

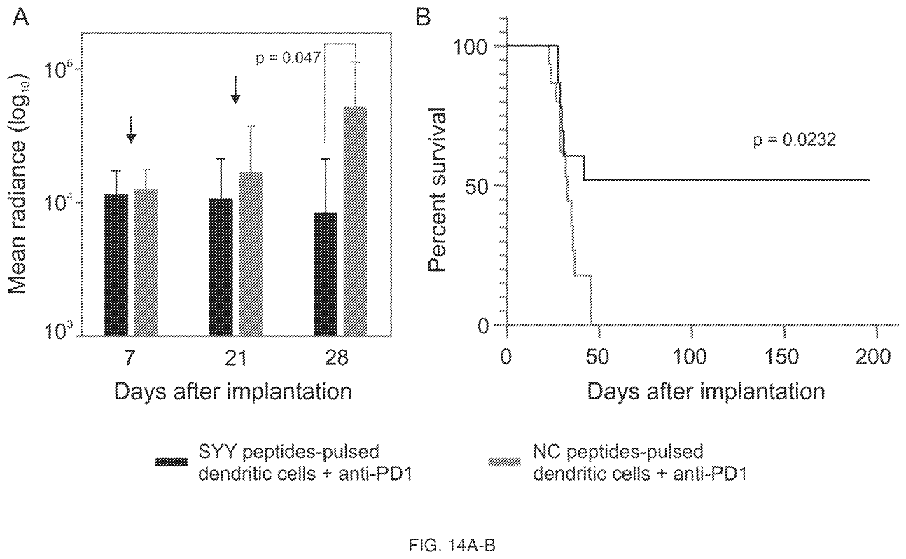

Having established that DC vaccination with tumor lysate was a feasible treatment modality in the H3G34R HGG model, the inventors next investigated whether a peptide vaccine would also provide a survival benefit. To identify potential peptide candidates, the inventors obtained RNA sequencing data from an H3G34R tumor specimen taken from a patient who underwent resection at UCLA. Since the patient was HLA-A*02:01-positive, the inventors focused on peptides that were predicted to bind with high affinity to this HLA class I allele. The screen yielded 74 candidate nonapeptide epitopes that bound with a calculated Kd of <100 nM to HLA-A*02:01. Fifty-one peptide epitopes resulted from exon skipping, and 23 from exon inclusion events. Re-screening the peptides against the HLA class I alleles curated in the NetMHC database indicated that all of the peptides may bind with high affinity to at least one other HLA-A allele (FIG. 3; Table S2). The inventors focused on the three peptides that were also predicted to bind, with high affinity to the MHC class I allele H-2K^{b} which is carried by C57Bl/6J mice (Table 2). The inventors employed the T2 cell HLA stabilization assay, with the K27M peptide identified by Okada's group as a positive control, to verify that the three peptides bound HLA-A*0201 (FIG. 4), and confirmed that the three genes from which the peptide sequences were derived were expressed in implanted H3G34R/RCAS2 tumors (Table 2).

4. Treatment with Tripeptide-Pulsed Dendritic Cells and Anti-PD-1 Checkpoint Inhibitor

The inventors followed the same treatment protocol as described previously, replacing tumor lysate with the three peptides. Tumor growth was monitored by chemiluminescence over the first 28 days post implantation, and animal survival was recorded over 48 days. Animals in the control group received DCs pulsed with three H3.3 decapeptides that span glycine 34 (wild-type, G34R, and G34V sequences), and checkpoint blockade. None of these peptides were predicted to bind mouse MHC class I alleles (Table 2). As shown in FIG. 5 vaccination with SYY peptide pulsed DCs slowed tumor growth and decreased mortality compared with control group. During the course of the experiment, the inventors did not observe any immune pathology indicative of an autoimmune response, such as vitiligo. To investigate the cellular immune response, the inventors sacrificed three animals from each group seven days after the second DC injection, and prepared leukocytes from brain, lymph nodes and spleen from four animals, two from each group. Profiling these cells by flow cytometry showed that T cells in tumor infiltrating leukocytes (TILs) from the two animals treated with DC-pulsed SYY peptides comprised 43.2% CD8⁺ and 44.5% CD4⁺ T cells of which 69% and 45.3% respectively expressed PD-1 (FIG. 6A). In contrast, T cells in the TILs from the two animals treated with control group comprised fewer CD8⁺ T cells of which only 26.3% expressed PD-1 (FIG. 6C). There was about the same percentage of CD4⁺ T cells between the two groups however there were more double negative T cells in the control group. The inventors do not know whether these latter T cells were originally CD4⁺, although they suspect that they were because analysis of TILs from an untreated tumor-bearing animal showed that almost all of the CD3⁺ TILs expressed CD4 (FIG. S1). The inventors found that only a small fraction of the CD4⁺ T cells expressed CD25 (6.4% in the G34 peptide TILs, and 16.9% in the SYY peptide TILs), and the majority of these cells were also PD-1⁺ in the SYY peptide TILs (FIG. 2S). FoxP3 was not in the antibody panel, thus the inventors do not know how many of the CD25⁺ CD4⁺ T cells were likely to be Tregs. From the total number of TILs isolated, the inventors estimate that treatment with the therapeutic peptide vaccine resulted in approximately six times as many infiltrating T cells (Table S3).

Staining TILs with the myeloid antibody panel identified four different populations of CD3-CD11b⁺ cells (FIG. 6 and FIG. S3): Ly6C⁺ CD11c⁺ F4/80⁺ XCR1⁺, Ly6C⁺ CD11c⁺ F4/80⁺ XCR1⁻, Ly6C-CD11c⁺ F4/80⁺ XCR1⁻, Ly6C⁻ CD11c⁺ F4/80⁻ XCR1⁻. There was a relative increase in the percentage of CD11b⁺ Ly6C⁺ CD11c⁺ XCR1⁺ myeloid cells in the TILs from mice treated with the SYY peptides compared with TILs from mice treated the H3G34 peptides. Since XCR1 is a marker found on cross presenting conventional type 1 DCs, the inventors infer that these cells were likely to be monocyte-derived dendritic cells (moDCs) or macrophages that can cross-present MHC class I-specific peptides to CD8⁺ T cells. The Ly6C⁻ CD11c⁺ myeloid cells that did not express F4/80 were likely to be conventional type 2 DCs (FIG. S3). Based on the total TIL counts, the inventors estimate that there were about three times as many of myeloid cells in the TILs from animals treated with the SYY peptides (Table S3). To confirm these results, the inventors fixed the brain from the third animal from each group and performed multiplex immunofluorescence.

### B. Discussion

In this report, the inventors provide preclinical support for treating H3G34R/V HGG with DCs pulsed with identified TSAs combined with PD-1 mAb. In order to test this therapeutic modality, the inventors developed an H3G34R syngeneic tumor model using the RCAS/tv-A system. Transcriptome analysis of three independently derived non-clonal H3G34R lines showed that they expressed TFs that maintain neural stem cells in a proliferative uncommitted state, including SOX2, HES1, HES5, FOXG1, and OLIG2. OLIG2 expression persists in the oligodendrocyte lineage, but the phosphorylation state of OLIG2 determines whether it promotes the proliferation of neural progenitors or oligodendrocyte differentiation. It has been reported that OLIG2 is not expressed in H3G34R tumors, as a result of DNA methylation-induced transcriptional silencing. However, HGG characterized by PDGFRA amplification do express OLIG2, suggesting that the expression of OLIG2 in the mouse model, and possibly OLIG1 and SOX10 are a consequence of the forced overexpression of PDGF-B. Recent reports provide evidence that the cell of origin of H3G34R tumors may be a ventral forebrain interneuron progenitor. The H3G34R cell lines that the inventors generated also express proneural TFs that are required for the formation of interneurons, namely ASCL1 and GSX1. The cells expressed much less GSX2 (Table S2), a second closely related homeodomain TF that is also required for interneuron differentiation. Based on the close proximity of GSX2 to PDFGRA on chromosome 4 recent evidence has been presented to explain how aberrant expression of PDGFRA may occur in an interneuron progenitor leading to oncogenesis. Single-cell RNA sequencing will resolve whether the same cells express TFs for determining both interneuron and oligodendrocyte lineages in the H3G34R model.

The inventors identified TSAs that result from alternative RNA splicing (AS) events that occur more frequently in tumor cells than in comparable normal tissues. Candidate nonamers were selected based on binding to HLA-A*02:01 with a predicted K_{d}<100 nM, and resulted from exon skipping or exon inclusion events, and not from aberrant intron retention. Three of the peptides were also predicted to bind H2-K^{b} with affinities <100 nM, which allowed us to test their therapeutic efficacy in the H3G34R model. The inventors found a statistically significant increase in survival after treatment with DCs pulsed with the three peptides in combination with anti-PD-1 checkpoint inhibitor. The failure of PD-1 blockade combined with DCs pulsed with negative control peptides not predicted to bind H2-K^{b} provides evidence that the therapeutic effect that the inventors observed was directly attributable to the peptides that the inventors selected based on their predicted binding to H2-K^{b}. Profiling isolated TILs from tumor-bearing animals showed that there were more T cells and monocytes in the TME after exposure to the candidate therapeutic peptides compared with peptides that were not predicted to bind mouse MHC class I alleles even though both groups received a checkpoint inhibitor. This observation indicates that animals treated with the therapeutic peptides mounted a more efficacious cellular immune response which led to an increase in the number of TILs, and a positive therapeutic outcome. The three peptides are self-antigens resulting from AS events; thus, the inventors assume that the CD8⁺ T cells that can recognize them must have escaped central and peripheral tolerance mechanisms. There were CD4+ and CD8⁺ T cells in the TME of treated and control animals, and both subsets expressed PD-1, indicating that they could be made up of terminal exhausted effector T cells (TEX) and/or precursor exhausted T cells (TPEX). Irrespective of whether animals were treated with DC vaccine and PD-1 mAb, CD4⁺ T cells were found in the TME in the tumor model (FIG. 1S). Only a small fraction of CD4⁺ T cells in treated animals expressed CD25, thus may be Tregs, whereas the majority of T cells likely comprise other subsets that may promote or suppress an anti-tumor immune response. The importance of CD4⁺ T cells in the TME has been known for some time, and has guided the design of cancer peptide vaccines that engage CD4⁺ T cells as well as CD8⁺ T cells. In the H3G34R model, the presence of CD4⁺ T cells in treatment naïve tumor-bearing animals does not appear to provide any survival benefit; however, after DC vaccination, CD4⁺ T cells in the TME could be contributing to the therapeutic efficacy of CD8⁺ T cells that infiltrate the tumor.

The majority of myeloid cells in TILs from both SYY peptide and negative control peptide-treated animals were Ly6C⁺, attesting to their monocytic origin. Based on the marker panel the inventors cannot distinguish between monocyte-derived DCs (moDCs) or inflammatory DCs on the one hand, and monocyte derived myeloid derived suppressor cells (moMDSCs) or tumor-associated macrophages (TAMs) on the other. However, a recent CITEseq analysis of myeloid cells in a murine HGG model suggests that the cells are likely to be transitory TAMs. The expression of XCR1 by a large fraction of the cells suggests that they may promote an anti-tumor CTL response. XCR1 is a marker for cross presenting conventional type 1 DCs and it has been shown that both moDCs and macrophages in humans can cross-present antigens to CD8⁺ T cells. Single-cell RNA will provide more information about the functionality of the myeloid populations in the TME. Likewise, RNAseq combined with single-cell TCR sequencing will allows us to better define the CD8⁺ and CD4⁺ subsets in the TME in treatment naïve and DC vaccine-treated animals, and determine the extent of clonal restriction. Single-cell TCR sequencing will also provide us with the sequence information to clone individual TCRs to directly test their reactivity to the therapeutic peptides and cytotoxic function ex vivo.

In summary, in a preclinical model, the inventors have demonstrated the immunotherapeutic benefit of DC vaccination targeting TSAs derived from AS events that are enriched in the transcriptome of H3G34R HGG.

### C. Materials and Methods

### 1. Derivation of a H3G34R RCAS/Tv-a Model

All of the animal experiments were approved by the UCLA Institutional Animal Care and Use Committee. Surgical procedures were carried out in a BSL2 biological safety cabinet. G34R HGG were generated essentially as described by Misuraca et al., Postnatal day 2-3 pups were anesthetized with a mixture isofluorane/oxygen delivered through a nose conc trimmed to fit the neonate. Two microliters of packages cells comprising equal numbers of DF1 cells producing RCAS viruses encoding H3G34R, Cre recombinase, and PDGF-B in phosphate-buffered saline (PBS) were injected (1×10⁵ cells per μl) into the left lateral ventricle through a 28-gauge needle attached to a 101 Hamilton syringe. The needle penetrated the skull to a depth of ˜2 mm at a location ˜0.25 mm lateral to the sagittal suture and 0.5-0.75 mm rostral to the neonatal coronary suture. Animals were returned to their home cages and monitored daily after weaning for signs of tumor formation as evidenced by loss of body weight and problems with locomotion at which point the animal was euthanized with C0₂, and the brain removed. A Brain Tumor Dissociation Kit (P) (Miltenyi Biotec Inc., Auburn, CA) was used to dissociate the mouse brains, which were then fractionated on a 70%/30% Percoll® gradient (Sigma-Aldrich, St. Louis, MO) to the remove myelin. Cells were placed in culture in medium formulated to support the growth of human pediatric HGG. All injected animals were sacrificed after twelve weeks.

### 2. Orthotopic Delivery of Tumor Cells and Treatment Protocol

Under ketamine/xylazine anesthesia, a burr hole was drilled in the skull 2 mm lateral to Bregma and 0.5 mm caudal and injections of 1×10⁵ H3G34R RCAS tumor cells in 2 μL of PBS were made into the left hemispheres of eight-week-old syngeneic C57Bl/6J female mice. Cells were injected through a 28-gauge needle attached to a 10 μL Hamilton syringe at a depth of 2.5 mm. DCs were prepared from bone marrow (BM) essentially as previously described. In brief, BM cells were plated in RPMI-1640 plus 10% fetal bovine serum (FBS) with antibiotics, and after 24 hours the nonadherent cells were counted and re-plated in 12-well tissue culture plates (˜2×10⁶ cells per well) in RPMI-1640 plus 10% FBS supplemented with murine granulocyte-macrophage colony stimulating factor (GM-CSF, 100 ng/ml; Biolegend, San Diego, CA) and interleukin-4 (IL-4, 500 U/ml; Biolegend). The medium was replaced after three days, and three days later the adherent differentiated DCs were pulsed overnight with 250 μg per well of a freeze thawed lysate of H3G34R RCAS2 cells or 10 μg of each peptide per well. Cells were harvested, washed, resuspended in PBS and 1×10⁶ cells were injected intradermally seven days after tumor implantation together with an IP injection of anti-mouse PD-1 (250 μg per animal; clone RMP1-14; Bio X Cell, Lebanon, NH). PD-1 mAb was re-administered 48 hours later. The same treatment regimen was repeated 21 days after tumor implantation. In vivo imaging was performed under isoflurane anesthesia after IP injection of luciferin (100 μl of 5 mg/ml stock solution). Chemiluminescence images were captured using an IVIS Lumina II imaging system (Perkin Elmer, Waltham, MA). Log transformed mean radiance values and standard errors, and survival data were analyzed and plotted in GraphPad Prism (GraphPad Software Corp., San Diego, CA). Graphs were exported as enhanced metafiles to CorelDraw2017 (Corel Corporation, Ottawa, Canada).

### 3. DNA Sequencing

Genomic DNA was isolated from cultured RCAS tumor cells using a commercially available kit (Quick-DNA miniprep kit, Zymo Research, Irvine CA). To confirm the presence of the G34R mutation, PCR primers flanking the cloning site in the RCAS vector were used to amplify inserted sequences (forward primer 5′ GTCTGTGTGCTGCAGGAGCTGAGCTGACTCTGCTG 3′ (SEQ ID NO:108), reverse primer 5′ GATACGCGTATATCTGGCCCGTACATCGCATCG 3′ (SEQ ID NO:109)), which were treated with ExoSAP-IT™ (ThermoFisher, Waltham, MA) then sequenced using an H3.3-specific primer (5′GCACGTTCTCCACGTATGCGGCGTG 3′ (SEQ ID NO:110)).

### 4. RNA Sequencing and IRIS Pipeline

Total RNA was extracted from human and mouse tumors and cells using commercially available kits either RNAeasy kit (Qiagen, Germantown, MD) or Direct-zol RNA kit (Zymo Research). The library constructed from the human tumor LB3570 was pair-end sequenced (100 bp). The mouse tumors were single-end sequenced (50 bp). Multivariate Analysis of Transcript Splicing (MATS) with a statistically robust filtering step to identify splice variants that are over-represented or exclusively expressed in the tumor transcriptome. The heat map of calculated affinities determined by NetMHC was generated using Morpheus software (www.broadinstitute.org) and exported to CorelDraw2017 as a scalable vector graphic (svg) filc.

### 5. Flow Cytometry

A leukocyte fraction was isolated from tumor-bearing animals by dissociating brain tissue as described above and fractionating on a 30%: 70% Percoll® gradient. Isolated lymph nodes and spleens were macerated in RPMI-1640 and strained through a 70-micron sieve. Cells were labeled with the following two antibody panels: lymphoid panel-CD45 (Alexa Fluor (AF) 700-conjugated; clone 30-F11), CD3 (Allophycocyanin (APC)-Cyanine 7 (Cy7)-conjugated; clone 145-2C11), CD4 (APC-conjugated; clone RM4-5), CD8 (Phycocrythrin (PE)-Cy7-conjugated; clone 53-6.7), CD25 (Brilliant Violet (BV) 650-conjugated; clone PC61), PD-1 (PE-conjugated; clone RMP1-14), CD45R/B220 (Peridinin chlorophyll protein (PerCP)-Cy5.5-conjugated; clone RA3-6B2), and NK1.1 (Fluorescein isothiocyanate (FITC)-conjugated; clone PK136), plus Zombie Violet™; myeloid panel-CD45 (AF700-conjugated), CD3 (APC-Cy7-conjugated), CD11b (PE-conjugated; clone M1/70), CD11c (PerCP-Cy5.5 conjugated; clone N418), Ly6C (BV650-conjugated; clone Hk1.4), Ly6G (PE-Cy-conjugated; clone RB6-8C5), F4/80 (FITC-conjugated; clone BM8), and XCR1 (APC-conjugated; clone ZET) plus Zombie Violet™. All antibodies were purchased from Biolegend, and samples were run on a LSRII analytical flow cytometer (Becton Dickinson, Franklin Lakes, NJ). Individual FCS files were analyzed using FlowJo® software (FlowJo LLC, Ashland, OR); 2-D contour plots were exported as svg files to CorelDraw2017.

### 6. T2 Cell HLA Stabilization Assay

HLA-A*02:01 TAP deficient T2 cells (2×10⁵ in 200 μL) were incubated with peptides (25 μg per ml in 10% DMSO in PBS) in AIM-V plus 1% human serum at 25° C. overnight to facilitate peptide binding by reducing HLA turnover. The following day the cells were returned to 37° C. for 5 hours then stained with a pan HLA antibody (FITC-conjugated HLA, B, C; clone W6/32). HLA class I expression was measured by flow cytometry, and the percent increase in mean fluorescence intensity over cells incubated with 10% DMSO alone was calculated.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### D. Tables

TABLE 2
Calculated affinities of peptides and gene expression.

| | | H-2 kb | |
|---|---|---|---|
| | | affinity | Gene |
| Gene | Peptide | (nM)* | expression^{¶} |
| Arhgap4 | YLFTFLNHL | 55.84 | 53.88 ± 1.22 |
| | (SEQ ID NO: 39) | | |
| At12 | YMYNKVAVL | 53.98 | 9.94 ± 0.62 |
| | (SEQ ID NO: 33) | | |
| Sfxn5 | SMLEKTALL | 86.5 | 56.93 ± 8.93 |
| | (SEQ ID NO: 32) | | |
| H3-3a | KSAPSTGRVK | 27883.23 | 12.22 ± 0.22 |
| | (SEQ ID NO: 11) | | |
| | KSAPSTGVVK | 34052.35 | |
| | (SEQ ID NO: 9) | | |
| | KSAPSTGGVK | 31141.75 | |
| | (SEQ ID NO: 35) | | |

*Affinities predicted by NetMHC4.0
^{¶}TMM normalized counts (n = 3, mean ± SEM)

TABLE S2
Predicted binding affinities of tumor-associated antigens to HLA class I alleles

| | SEQ | | | | | | | | | | | |
|---|---|---|---|---|---|---|---|---|---|---|---|---|
| | ID | HLA- | HLA- | HLA- | HLA- | HLA- | HLA- | HLA- | HLA- | HLA- | HLA- | HLA- |
| Peptide | NO: | A*0201 | A*0202 | A*0203 | A*0205 | A*0206 | A*0211 | A*0212 | A*0216 | A*0217 | A*0219 | A*0250 |
| ALACDWWFL | 37 | 5.84 | 3.75 | | | 4.82 | 3.58 | 7.72 | 3.98 | 55.66 | 11.01 | 3.68 |
| ALFGDVKFV | 38 | 12.87 | 8.03 | 5.89 | | | 2.84 | 11.92 | 3.87 | | 7.71 | 4.58 |
| ALLDEVLDV | 39 | 8.09 | | 17.37 | | 19.35 | 8 | 13.15 | 6.11 | | 19.85 | 4.07 |
| ALLPCCNRV | 40 | 15.61 | 17.09 | | | 15.87 | | 29.36 | 56.71 | | 52.59 | 5.69 |
| ALLYSAFGV | 41 | 5.49 | | | | 6.48 | 2.66 | 3.66 | 4.31 | 68.01 | 10.46 | 3.42 |
| ALMNGLIMT | 42 | 20.18 | | 16.83 | | | 5.96 | 70.74 | 25.19 | | 37.56 | 19.48 |
| AMLTVIPEV | 43 | 2.94 | | 11.27 | | 4.91 | 2.02 | 3.29 | 2.13 | 72.34 | 4.9 | 9.98 |
| AMYSVEITV | 44 | 7.03 | 11.19 | 10.33 | | | 1.68 | 5.25 | 2.67 | | 54.92 | 9.96 |
| CMMISVWNL | 45 | 14.84 | | | | | 2.39 | 2.92 | 4.45 | 42.19 | 3.39 | 5.53 |
| FIFEHSYSV | 46 | 2.77 | 2.48 | 2.23 | | 2.17 | 1.89 | 2.27 | 2.54 | 30.13 | 3.95 | 4.52 |
| FISEIGPAV | 47 | 6.72 | 4.98 | 4.54 | | 3.61 | 2.83 | 7.04 | 6.87 | | 39.84 | 12.54 |
| FISSCSLPV | 48 | 4.93 | 4.04 | 3.59 | | 4.24 | | 27.11 | 14.94 | | 48.35 | |
| FLALCATGL | 49 | 16.07 | 3.3 | 5.58 | | 22.75 | | 32.81 | 61 | | 11.85 | 5.39 |
| FLFERVEGI | 50 | 5.29 | 3.29 | 2.77 | | 6.35 | 1.69 | 3.88 | 4 | 88.26 | 4.97 | 14.4 |
| FLFGVDEYL | 51 | 5.05 | 2.23 | 10.75 | | 8.28 | 1.85 | 5.75 | 2.24 | 41.82 | 4 | 11.6 |
| FLGPVIVEI | 52 | 7.2 | 4.62 | 4.86 | | 24.84 | 1.69 | 2.86 | 2 | 48.67 | 6.97 | 7.73 |
| FLIGFGLWL | 53 | 8.12 | 7.05 | 11.63 | | | 4.59 | 23.27 | 17.12 | 59.96 | | 7.67 |
| FLLDLDPLL | 54 | 3.08 | 6.33 | | | 3.88 | 3.98 | 3.94 | 2.14 | 22.18 | 4.41 | 6.69 |
| FLLRKVFPL | 55 | 2.94 | 7.65 | 15.79 | | 5.3 | 2.16 | 2.25 | 2.38 | 10.48 | 3 | 15.03 |
| FLQATDFVV | 56 | 6.23 | 8.37 | | | 12.89 | 2.83 | 2.24 | 2.89 | 45.58 | 4.4 | 4.18 |
| FLSDLNLLV | 57 | 2.79 | 2.15 | 2.86 | | 5.24 | 3.4 | 6.04 | 6.23 | | 6.69 | 4.95 |
| FLSDTQVFV | 58 | 3.14 | 2.16 | 2.52 | | 4.66 | 1.96 | 2.28 | 2.3 | 90.9 | 2.09 | 3.23 |
| FLSDVKDGV | 59 | 9.87 | 3.15 | 7.16 | | 7.73 | 2.36 | 5.55 | 3.41 | | 4.85 | 3.95 |
| FLVMYSHFA | 60 | 8.93 | 4.76 | 5.56 | | 10.76 | 6.27 | 4.13 | 4.45 | | 16.07 | 3.59 |
| FLVSGIAKV | 61 | 3.79 | 4.51 | 1.98 | 54.63 | 4.14 | 2.14 | 3.34 | 2.61 | 64.31 | 4.38 | 4.8 |
| FLYMDYLVL | 62 | 18.68 | | | | | 14.63 | 32.73 | 16.67 | | | 4.45 |
| FQILSVVPV | 63 | 13.94 | | 7.19 | | 2.93 | 7.68 | 7.78 | 12.81 | | | |
| GLIYFFVQV | 64 | 15.54 | 12.29 | 16.03 | | 16.42 | 6.52 | 12.03 | 21.84 | | | 21.15 |
| GLWEEAYRL | 65 | 7.61 | | | | | 4.69 | 4.38 | 6.57 | | 10.9 | 8.1 |
| GMLQMDWEV | 66 | 4.34 | | | | 7.02 | 2.84 | 2.5 | 1.86 | 80.48 | 4.48 | 2.88 |
| ILLDCQYLA | 67 | 6.64 | 6.83 | | | 23.3 | 14.6 | 12.15 | 11.03 | | 9.77 | 17 |
| IMSAVPFLI | 68 | 8.1 | 3.42 | | | | 2.59 | 56.83 | 38.58 | | 29.83 | |
| KIIHWPWLV | 69 | 4.01 | | 9.06 | | 10.51 | 3.77 | 12.02 | 7.41 | 61.79 | | 14.39 |
| KLDMGTTLV | 70 | 10.76 | | | | 18.21 | | 60.05 | 33.74 | | 44.76 | 23.46 |
| KLRSWMYAV | 71 | 9.55 | 8.8 | 4.09 | 76.09 | 23.46 | 2.08 | 5.61 | 7.66 | 86.6 | 67.28 | 18.9 |
| KMLDKLRYV | 72 | 4.18 | 15.88 | 3.37 | | 5.51 | 1.72 | 3.15 | 3.65 | | 3.24 | 5.11 |
| KMLVDCVPL | 73 | 15.05 | | | | 19.4 | 11.4 | 21.44 | 27.42 | 74.55 | | 38.77 |
| KMYKTPIFL | 74 | 11.18 | 17.79 | | | | 2.39 | 6.19 | 9.04 | | 63.38 | 29.11 |
| LLFSGCAGL | 75 | 10.46 | 4.93 | 5.56 | | | 6.89 | 39.92 | 10.56 | 39.18 | 12.61 | 12.83 |
| LLLAIMSAV | 76 | 6.99 | 7.11 | 5.22 | | 8.13 | 2.68 | 8.85 | 22.79 | 61.65 | 7.9 | 22.17 |
| LLMVLPFLA | 77 | 11.5 | 19.07 | | | | | | | | | |
| LMNGLIMTV | 78 | 7.03 | 6.22 | 3.15 | | | 1.75 | 2.85 | 2.98 | | 3.87 | 4.53 |
| MLADIPVTI | 79 | 5.1 | 2.62 | 2.81 | | | 2.54 | 10.32 | 20.43 | | 9.06 | 15.38 |
| MLFHSYPPA | 80 | 5.55 | 12.37 | 3.88 | | 6.39 | 10.37 | 42.82 | 5.62 | | | |
| MLFNDAIRL | 81 | 11.67 | 12.07 | | | | 10.66 | | 30.79 | | 90.82 | 36.6 |
| MMKAAMYSV | 82 | 9.02 | 3.5 | 2.75 | | 12.55 | 2.49 | 4.26 | 15.19 | 19.85 | 23.18 | 6.41 |
| NLLAEIHGV | 83 | 7.61 | | 13.24 | | 8.65 | 2.78 | 3.02 | 3.82 | | 3.16 | 3.89 |
| QLLDLFYIL | 84 | 14.29 | | | | 12.63 | | 9.64 | 25.82 | | 34.74 | 11.04 |
| QLLYWFLKV | 85 | 15.11 | | | | 13.88 | 7.09 | 52.28 | 22.19 | | | 6.69 |
| SLMDKLLPV | 86 | 2.12 | 3.22 | 1.77 | | 2.51 | 2.12 | 2.33 | 5.55 | 53.03 | 2.74 | 8.07 |
| SMLEKTALL | 32 | 8.33 | 10.28 | 15.63 | | | 3.64 | 3.78 | 9.73 | | 10.18 | 28.66 |
| TMWDYTIPI | 87 | 2.5 | 9.18 | 9.46 | | 6.21 | 1.89 | 2.75 | 4.72 | 75.52 | 3.92 | 15.04 |
| VLWNGIPTA | 88 | 6.03 | | | | | 3.1 | 17.74 | 3.3 | | 7.58 | 27.4 |
| VLYTIFMKV | 89 | 9.97 | | 6.01 | | | 4.8 | 24.11 | | | 57.14 | 12.89 |
| VMDNLLIQV | 90 | 8.76 | | | | 14.92 | 1.79 | 4.98 | 3.4 | | 6.78 | 24.24 |
| VQWDLLHGV | 91 | 6.34 | | | | 2.9 | 2.23 | 4.87 | 9.37 | | 5.53 | 7.01 |
| WLLISVWGL | 92 | 15.26 | | | | | 3.16 | 5.26 | 2.55 | 38.37 | 4.15 | 3.52 |
| WMAPEVAAV | 93 | 7.24 | 4.68 | 2.46 | | 5.28 | 2.53 | 3.72 | 6.99 | | 6.49 | 2.31 |
| WVLELPYFV | 94 | 5 | | | | 6.34 | 2.69 | 4.58 | 3.96 | | 3.59 | 10.56 |
| YLAAFRFWI | 95 | 4.21 | 2.85 | 9.5 | | 20.3 | 3.86 | 47.59 | 23.58 | 69.08 | 50.78 | 8.52 |
| YLAKMSLSV | 96 | 3.8 | 3.89 | 2.2 | 24.31 | 7.25 | 3.95 | 5.89 | 4.86 | 60.14 | 5.79 | 3.35 |
| YLDGIITIV | 97 | 4.35 | 6.7 | 5.29 | | 13.11 | 2.18 | 4.21 | 3.07 | | 3.9 | 4.12 |
| YLFNSVVNV | 98 | 2.97 | 5.77 | 2.31 | | 7.84 | 1.71 | 2.69 | 2.08 | | 3.38 | 6.17 |
| YLFTFLNHL | 39 | 4.48 | 2.94 | 3.33 | | 8.45 | 2.93 | 8.49 | 18.69 | 38.54 | 14.21 | 11.8 |
| YLFVQPDYI | 99 | 9.63 | 3.65 | 11.11 | | | 3.64 | 9.34 | 7.99 | | 31.79 | 30.75 |
| YLLMVLPFL | 100 | 3.21 | | | | 6.79 | 2.48 | 4.88 | 2.54 | 9.71 | 3.42 | 6.9 |
| YLMGVGALA | 101 | 8.7 | 4.52 | 3.1 | | 18.95 | 3.43 | 8.09 | 11.23 | | 8.12 | 4.88 |
| YLQAYSATV | 102 | 3.76 | 4.62 | 3.01 | 8.79 | 10.77 | 1.76 | 1.87 | 3.85 | 49.29 | 4.19 | 2.57 |
| YLSTIVTEV | 103 | 2.96 | 2.76 | 1.97 | 87.03 | 11.9 | 4.99 | 6.22 | 7.36 | | 4.46 | 6.62 |
| YLWFLCRYL | 104 | 12.73 | | | | | 3.21 | 74.97 | 30.39 | | 13.66 | 8.22 |
| YLWLIYCYL | 105 | 6.5 | 11.62 | | | | 1.96 | 5.52 | 3.31 | 42.68 | 3.2 | 2.74 |
| YMDYLVLVL | 106 | 9.45 | | | | | 3.14 | 9.93 | 12.42 | | 78.73 | 17.69 |
| YMRCCLWKL | 107 | 13.57 | 6.91 | 8 | | 15.06 | 2.44 | 5.27 | 5.17 | 69.2 | 20.16 | 3.13 |
| YMYNKVAVL | 33 | 9.31 | 14.54 | 2.98 | | | 1.89 | 3.04 | 3.94 | | 5.44 | 5.02 |

TABLE S2
Predicted binding affinities of tumor-associated antigens to HLA class I alleles (continued)

| | SEQ | | | | | | | | | | | |
|---|---|---|---|---|---|---|---|---|---|---|---|---|
| | ID | HLA- | HLA- | HLA- | HLA- | HLA- | HLA- | HLA- | HLA- | HLA- | HLA- | HLA- |
| Peptide | NO: | A*2301 | A*2402 | A*3001 | A*3201 | A*3207 | A*6802 | A*6823 | A*6901 | B*801 | B*1501 | B*1503 |
| ALACDWWFL | 37 | | | | | | | | | | | |
| ALFGDVKFV | 38 | | | | | | | | | | | |
| ALLDEVLDV | 39 | | | | | | | | | | | |
| ALLPCCNRV | 40 | | | | | | | | | | | |
| ALLYSAFGV | 41 | | | | | | | | 95.95 | | | |
| ALMNGLIMT | 42 | | | | | | | | | | | |
| AMLTVIPEV | 43 | | | | | | | | | | | |
| AMYSVEITV | 44 | | | | | | | | | | | |
| CMMISVWNL | 45 | | | | | | | | | | | |
| FIFEHSYSV | 46 | | | | 93.05 | 92.75 | 5.84 | | 4.18 | 31.16 | | |
| FISEIGPAV | 47 | | | | | | | | 20.04 | | | |
| FISSCSLPV | 48 | | | | | | 10.17 | | 76.72 | | | |
| FLALCATGL | 49 | | | | | | | | | | | |
| FLFERVEGI | 50 | | | | | | | | | | | |
| FLFGVDEYL | 51 | | | | | | | | | | | |
| FLGPVIVEI | 52 | | | | | | | | | | | |
| FLIGFGLWL | 53 | | | | | | | | | | | |
| FLLDLDPLL | 54 | | | | | | | | | | | |
| FLLRKVFPL | 55 | | | | | | | | | 4.21 | | |
| FLQATDFVV | 56 | | | | | | | | | | | |
| FLSDLNLLV | 57 | | | | | | | | | | | |
| FLSDTQVFV | 58 | | | | | | 33.65 | | | | | |
| FLSDVKDGV | 59 | | | | | | 20.51 | | | | | |
| FLVMYSHFA | 60 | | | | | | | | | | | |
| FLVSGIAKV | 61 | | | | | | | | 35.16 | | | |
| FLYMDYLVL | 62 | | | | | | | | | 56.73 | | 14.66 |
| FQILSVVPV | 63 | | | | | | | | | | | 10.13 |
| GLIYFFVQV | 64 | | | | | | | | | | | |
| GLWEEAYRL | 65 | | | | | | | | | | | |
| GMLQMDWEV | 66 | | | | | 96.94 | | | | | | |
| ILLDCQYLA | 67 | | | | | | | | | | | |
| IMSAVPFLI | 68 | | 84.12 | | | | | | | | | |
| KIIHWPWLV | 69 | | | | 86.83 | 66.49 | | | 84.36 | | | |
| KLDMGTTLV | 70 | | | | | | | | | | | |
| KLRSWMYAV | 71 | | | | | 58.84 | | | | | | |
| KMLDKLRYV | 72 | | | | | | | | | | | 15.38 |
| KMLVDCVPL | 73 | | | | 71.6 | | | | | | | |
| KMYKTPIFL | 74 | | | | | | | | | | | 6.98 |
| LLFSGCAGL | 75 | | | | | | | | | | | |
| LLLAIMSAV | 76 | | | | | | | | | | | |
| LLMVLPFLA | 77 | | | | | | | | | | | |
| LMNGLIMTV | 78 | | | | | | | | | | | |
| MLADIPVTI | 79 | | | | 14.47 | | 11.4 | | 60.74 | | | |
| MLFHSYPPA | 80 | | | 30.3 | | 64.97 | | | | | | 24.54 |
| MLFNDAIRL | 81 | | | | | | | | | | | |
| MMKAAMYSV | 82 | | | 15.31 | | | 39.13 | | 28.14 | 77.67 | | |
| NLLAEIHGV | 83 | | | | | | | | 10.5 | | | |
| QLLDLFYIL | 84 | | | | | | | | | | | |
| QLLYWFLKV | 85 | | | | | | | | 20.5 | | | |
| SLMDKLLPV | 86 | | | | | | | | 69.3 | 42.14 | | |
| SMLEKTALL | 32 | | | | | | | | | | | |
| TMWDYTIPI | 87 | | | | 10.26 | | | | 4.2 | | | 9.32 |
| VLWNGIPTA | 88 | | | | | | | | | | | |
| VLYTIFMKV | 89 | | | | | | | | | | | |
| VMDNLLIQV | 90 | | | | | | | | | | | |
| VQWDLLHGV | 91 | | | | | | | | | | | |
| WLLISVWGL | 92 | | | | | | | | | | | |
| WMAPEVAAV | 93 | | | | | | | | | | | |
| WVLELPYFV | 94 | | | | | | 11.76 | | 2.84 | | | |
| YLAAFRFWI | 95 | | | | | | | | 33.08 | | | |
| YLAKMSLSV | 96 | | | | | | | | | 61.11 | | |
| YLDGIITIV | 97 | | | | | | | | | | | |
| YLFNSVVNV | 98 | | | | | | | | 61.94 | | | |
| YLFTFLNHL | 39 | | | | | | | 84.2 | | | | |
| YLFVQPDYI | 99 | | | | | | | | | | | |
| YLLMVLPFL | 100 | | | | | | | | | | | |
| YLMGVGALA | 101 | | | | | | | | | | | |
| YLQAYSATV | 102 | | | | | | | | 38.86 | | | |
| YLSTIVTEV | 103 | | | | | | | | | | | |
| YLWFLCRYL | 104 | | | | | | | 75.11 | | | | |
| YLWLIYCYL | 105 | | | | | | | | | | | |
| YMDYLVLVL | 106 | | | | | | | | | | | |
| YMRCCLWKL | 107 | 92.11 | | | | | | | | | | 32.69 |
| YMYNKVAVL | 33 | | | | | 62.31 | | | | 22.3 | 35.38 | 4.95 |

TABLE S2
Predicted binding affinities of tumor-associated antigens to HLA class I alleles (continued)

| | SEQ | | | | | | | | | | | |
|---|---|---|---|---|---|---|---|---|---|---|---|---|
| | ID | HLA- | HLA- | HLA- | HLA- | HLA- | HLA- | HLA- | HLA- | HLA- | HLA- | HLA- |
| Peptide | NO: | B*2720 | B*3901 | B*4013 | B*5801 | B*5401 | C*303 | C*501 | C*602 | C*1203 | C*1402 | C*1502 |
| ALACDWWFL | 37 | | | | | | | | | | | |
| ALFGDVKFV | 38 | | | | | | | | | | | |
| ALLDEVLDV | 39 | | | | | | | | | | | |
| ALLPCCNRV | 40 | | | | | | | | | | | |
| ALLYSAFGV | 41 | | | | | | | | | | | |
| ALMNGLIMT | 42 | | | | | | | | | | | |
| AMLTVIPEV | 43 | | | | | | | | | | | |
| AMYSVEITV | 44 | | | | | | | | | | | |
| CMMISVWNL | 45 | | | | | | | | | | | |
| FIFEHSYSV | 46 | 31.14 | 91.46 | | | 80.79 | | | 54.27 | 5.44 | | 37.39 |
| FISEIGPAV | 47 | | | | | | | | | 44.75 | | |
| FISSCSLPV | 48 | | | | | | | | | 91.53 | | |
| FLALCATGL | 49 | | | | | | | | | | | |
| FLFERVEGI | 50 | | | | | | | | | 37.49 | | |
| FLFGVDEYL | 51 | | | | | | 23.49 | | | | | |
| FLGPVIVEI | 52 | | | | | | | | | 85.73 | | |
| FLIGFGLWL | 53 | | | | | | 52.38 | | | | | |
| FLLDLDPLL | 54 | | | | | | | | | | | |
| FLLRKVFPL | 55 | | | | | | 35.06 | | | | | |
| FLQATDFVV | 56 | | | | | | | | | | | |
| FLSDLNLLV | 57 | | | | | | | | | | | |
| FLSDTQVFV | 58 | | | | | | | | | 99.38 | | |
| FLSDVKDGV | 59 | | | | | | | | | | | |
| FLVMYSHFA | 60 | | | | | | | | | | | |
| FLVSGIAKV | 61 | | | | | | | | | 99.02 | | |
| FLYMDYLVL | 62 | | | | | | 17.65 | | | | 15.9 | |
| FQILSVVPV | 63 | 38.95 | 11.49 | | | | | | | 85.81 | | |
| GLIYFFVQV | 64 | | | | | | | | | | | |
| GLWEEAYRL | 65 | | | | | | | | | | | |
| GMLQMDWEV | 66 | | | | | | | | | | | |
| ILLDCQYLA | 67 | | | | | | | | | | | |
| IMSAVPFLI | 68 | | | | 20.8 | | | | | | | |
| KIIHWPWLV | 69 | | | | | | | | | | | |
| KLDMGTTLV | 70 | | | | | | | 12.34 | | | | |
| KLRSWMYAV | 71 | | | | | | | | | | | |
| KMLDKLRYV | 72 | | | | | | | | | | | |
| KMLVDCVPL | 73 | 62.22 | | | | | | | | | | |
| KMYKTPIFL | 74 | | | 74.23 | | | | | | | 80.69 | |
| LLFSGCAGL | 75 | | | | | | | | | | | |
| LLLAIMSAV | 76 | | | | | | | | | | | |
| LLMVLPFLA | 77 | | | | | | | | | | | |
| LMNGLIMTV | 78 | | | | | | | | | | | |
| MLADIPVTI | 79 | | | | | | | | | 49.33 | | |
| MLFHSYPPA | 80 | | | | | 42.77 | | | | | | |
| MLFNDAIRL | 81 | | | | | | | | | | | |
| MMKAAMYSV | 82 | | | | | | | | | | | |
| NLLAEIHGV | 83 | | | | | | | | | | | |
| QLLDLFYIL | 84 | | | | | | | | | | | |
| QLLYWFLKV | 85 | | | | | | | | | | | |
| SLMDKLLPV | 86 | | | | | | | | | | | |
| SMLEKTALL | 32 | | | | | | | | | | | |
| TMWDYTIPI | 87 | 4.83 | | 52.77 | | | | | | | 57.96 | |
| VLWNGIPTA | 88 | | | | | | | | | | | |
| VLYTIFMKV | 89 | | | | | | | | | | | |
| VMDNLLIQV | 90 | | | | | | | | | | | |
| VQWDLLHGV | 91 | 47.83 | | | | | | | | | | |
| WLLISVWGL | 92 | | | | | | | | | | | |
| WMAPEVAAV | 93 | | | | | | | | | 71.39 | | |
| WVLELPYFV | 94 | | | | | | | | | | | |
| YLAAFRFWI | 95 | | | | | | | | | | | |
| YLAKMSLSV | 96 | | | | | | | | | 88.31 | | |
| YLDGIITIV | 97 | | | | | | | 45.28 | | 42.63 | | |
| YLFNSVVNV | 98 | | | | | | | | | 10.87 | | |
| YLFTFLNHL | 39 | | | | | | | | | 23.62 | | |
| YLFVQPDYI | 99 | | | | | | | | | | | |
| YLLMVLPFL | 100 | | | | | | | | | | | |
| YLMGVGALA | 101 | | | | | | | | | | | |
| YLQAYSATV | 102 | | | | | | | | | 38.22 | 47.47 | |
| YLSTIVTEV | 103 | | | | | | | | | | | |
| YLWFLCRYL | 104 | | | | | | | | | | 81.58 | |
| YLWLIYCYL | 105 | | | | | | | | | | 22.06 | |
| YMDYLVLVL | 106 | | | | | | | 65.03 | | 43.43 | | |
| YMRCCLWKL | 107 | 70.46 | 9.89 | | | | | | | | | |
| YMYNKVAVL | 33 | 30.36 | 52 | | | | 4.92 | | | 8.75 | 5.83 | |

TABLE S3
Estimated total number of cells in CD45+ subsets.

| | Lymph nodes | | | Spleen | | | TILs | | |
|---|---|---|---|---|---|---|---|---|---|
| | SYY | G34 | ratio | SYY | G34 | ratio | SYY | G34 | ratio |
| Lymphoid cell panel | | | | | | | | | |
| Total CD45⁺ | 1.5 × 10⁷ | 1.49 × 10⁷ | 1.0 | 5.19 × 10⁷ | 4.93 × 10⁷ | 1.05 | 1.63 × 10⁶ | 4.57 × 10⁵ | 3.57 |
| CD3⁺ | 1.38 × 10⁷ | 1.32 × 10⁷ | 1.04 | 4.17 × 10⁷ | 3.66 × 10⁷ | 1.14 | 5.61 × 10⁵ | 8.97 × 10⁴ | 6.25 |
| CD8⁺ | 6.57 × 10⁶ | 6.33 × 10⁶ | 1.04 | 2.04 × 10⁷ | 1.43 × 10⁷ | 1.43 | 2.42 × 10⁵ | 1.78 × 10⁴ | 13.6 |
| CD8⁺ PD-1⁺ | 0 | 0 | | 0 | 0 | | 1.62 × 10⁵ | 4.78 × 10³ | 33.89 |
| CD4⁺ | 6.98 × 10⁶ | 6.73 × 10⁶ | 1.04 | 1.99 × 10⁷ | 2.09 × 10⁷ | 0.95 | 2.5 × 10⁵ | 4.46 × 10⁴ | 5.6 |
| CD4⁺ CD25⁺ | 1.63 × 10⁵ | 1.63 × 10⁵ | 1.0 | 4.78 × 10⁵ | 4.09 × 10⁵ | 1.17 | 1.13 × 10⁵ | 9.05 × 10³ | 12.49 |
| CD4⁺ PD-1⁺ | 0 | 0 | | 0 | 0 | | 4.55 × 10⁴ | 3.29 × 10³ | 13.83 |
| CD8⁻ CD4⁻ | 2.04 × 10⁵ | 1.45 × 10⁵ | 1.41 | 4.59 × 10⁵ | 1.20 × 10⁵ | 3.8 | 6.79 × 10⁴ | 2.59 × 10⁴ | 2.62 |
| CD3⁻ | 1.95 × 10⁶ | 5.93 × 10⁵ | 3.29 | 2.06 × 10⁶ | 1.23 × 10⁷ | 1.67 | 1.02 × 10⁶ | 3.62 × 10⁵ | 2.82 |
| B cells | 1.50 × 10⁶ | 4.29 × 10⁵ | 3.5 | 1.49 × 10⁶ | 9.33 × 10⁶ | 0.16 | 9.30 × 10⁴ | 1.08 × 10⁴ | 8.61 |
| NK cells | 2.00 × 10⁵ | 8.24 × 10⁴ | 2.43 | 2.87 × 10⁵ | 2.03 × 10⁶ | 0.14 | 3.55 × 10⁵ | 1.17 × 10⁵ | 3.03 |
| Myeloid cell panel | | | | | | | | | |
| Total CD45⁺ | 1.45 × 10⁷ | 1.56 × 10⁷ | 0.93 | 4.83 × 10⁷ | 5.10 × 10⁷ | 0.95 | 1.74 × 10⁶ | 4.41 × 10⁵ | 3.95 |
| CD3⁻ CD11b⁺ | 2.76 × 10⁴ | 2.96 × 10⁴ | 0.93 | 3.96 × 10⁶ | 1.6 × 10⁶ | 2.48 | 5.68 × 10⁵ | 1.92 × 10⁵ | 2.96 |
| Ly6C⁺ Ly6G⁻ | 8.11 × 10³ | 8.43 × 10³ | 0.96 | 1.69 × 10⁶ | 7.43 × 10⁵ | 2.27 | 4.52 × 10⁵ | 1.61 × 10⁵ | 2.81 |
| CD11c⁺ XCR1⁺ | 1.01 × 10² | 2.06 × 10² | 0.53 | 2.18 × 10⁴ | 2.97 × 10⁴ | 0.73 | 2.54 × 10⁵ | 8.41 × 10⁴ | 3.02 |
| CD11c⁺ XCR1⁻ | 7.09 × 10³ | 8.23 × 10³ | 0.86 | 1.66 × 10⁶ | 7.01 × 10⁵ | 2.37 | 1.94 × 10⁵ | 7.53 × 10⁴ | 2.58 |
| Ly6C⁻ Ly6G⁻ | 1.8 × 10⁴ | 1.97 × 10⁴ | 0.91 | 2.12 × 10⁶ | 8.04 × 10⁵ | 2.64 | 7.22 × 10⁴ | 1.40 × 10⁴ | 5.16 |
| CD11c⁺ XCR1⁻ F4/80⁺ | 4.31 × 10³ | 1.45 × 10³ | 2.96 | 1.38 × 10⁴ | 2.82 × 10⁴ | 0.49 | 3.32 × 10⁴ | 4.01 × 10³ | 8.27 |
| CD11c⁺ XCR1⁻ F4/80⁻ | 1.37 × 10⁴ | 1.83 × 10⁴ | 0.75 | 2.11 × 10⁶ | 7.76 × 10⁵ | 2.72 | 3.61 × 10⁴ | 9.15 × 10³ | 3.95 |

### Example 2—Targeting Alternative Splicing Variants to Stimulate Anti-Tumor Immunity in a Novel Syngeneic Mouse Model of Diffuse Hemispheric Glioma, H3 G34-Mutant

The data and description in Example 2 may be duplicative of data provided in Example 1.

Dendritic cell vaccination targeting alternative mRNA splicing variants enriched in the tumor transcriptome of diffuse hemispheric glioma, H3 G34-mutant patients stimulates anti-tumor immunity resulting in a durable increase in survival in a novel syngeneic mouse model.

The prognosis for pediatric high-grade gliomas associated with mutations in the H3-A3 histone gene is very poor. To investigate whether peptide-pulsed dendritic cells (DC) together with checkpoint blockade might be a potential treatment modality for diffuse hemispheric glioma, H3 G34-mutant, the inventors have developed a novel mouse model, and utilized a novel bioinformatics pipeline to identify tumor-associated peptide antigens. The inventors used the RCAS/tv-A system to target the expression of H3/G34R and PDGFB, and to knock out p53 in neural progenitors in transgenic C57BL/6 neonatal mice. Three independent cell lines were obtained that expressed transcripts associated with oligodendrocyte and interneuron lineages, and formed lethal tumors after intracranial implantation. To identify tumor-specific peptide antigens, the inventors implemented a new computational workflow to identify nonapeptides generated from alternative splicing of mRNAs that were highly enriched in the tumor transcriptomes of two H3/G34R patients, and predicted to bind the HLA-A*2:01 class I allele carried by these patients. Three of the candidate peptides were conserved in the mouse and also predicted to bind the H2-K^{b} MHC I allele carried by C57/BL6 mice. Using the syngeneic mouse model, the inventors showed that DCs pulsed with these peptides together with anti-PD1 mAb provided a significant survival benefit compared with checkpoint inhibitor and DCs pulsed with peptides that were not predicted to bind the H2-K^{b} allele.

### A. Introduction

High-grade gliomas (HGG) are among the most lethal of human cancers; median overall survival is typically under two years from initial diagnosis even with optimal multi-modal therapy. Recurrence is universal because tumors invade and infiltrate the surrounding brain making complete surgical excision impossible, while quiescent tumor-initiating cell populations evade adjuvant treatments. However, various forms of immunotherapy involving checkpoint blockade and active vaccination have shown promise in addressing the issue of recurrence in pre-clinical studies. HGG are generally relatively low in somatic mutational burden, which is frequently a negative predictor of response to immunotherapy, although not necessarily in HGG. The subtypes of HGG found in adults, and consequently the subtypes that have been more frequently studied in the context of clinical trials of immunotherapy, are dramatically heterogeneous and often involve copy number variability, without highly-conserved truncal mutations suitable for immune targeting. There are reasons to believe that the juvenile forms of HGG will respond differently to immunotherapy than adult forms of HGG, as they consistently carry a small number of oncogenic driver mutations.

In the present study, the inventors identified candidate TSAs from the tumor transcriptomes of two DHG patients that were predicted to bind HLA-A*0201. Three of the peptides, conserved in the mouse, were also predicted to bind the MHC I H-2K^{b} allele carried by C57BL6/J mice. Using the Replication-Competent Avian leukosis virus long terminal repeat with Splice acceptor (RCAS)/tumor virus-a (tv-a) system, the inventors generated syngeneic H3G34R tumor lines, which allowed us to test the efficacy of targeted dendritic cell (DC) vaccination in combination with anti-PD-1 checkpoint blockade in an immunocompetent pre-clinical model.

### B. Results

### 1. Generation of Syngeneic Murine H3G34R Gliomas

The inventors used the RCAS/tv-A system to develop an immunocompetent model of DHG. Intraventricular injections of DF-1 cells producing viruses encoding PDGF-B, Cre recombinase, and H3-3A G34R into postnatal day 2-3 C56B1/6J Tp53t1/11 Tg (Nestin-tv-A) mice were made, and three non-clonal tumor lines were established from three different animals. The presence of the H3G34R mutation in each line was confirmed by genomic PCR (FIG. 10A). From RNA sequencing, the inventors found that the three populations express similar levels of nestin (Nes) and PDGF-α receptor transcripts (Pdgfra), and mRNAs specifying transcription factor (TF) genes that maintain multipotent neural progenitors (FoxG1, Hes1, Hes5, Hey1, Sox2, and Olig2), as well as regulating neuronal (Ascl1, and Gsx1, Hes6, and Sox4), and oligodendrocyte lineages (Olig1, Olig2, and Sox10) of the ventral telencephalon (FIG. 10B; Table S1). Orthotopic delivery of 1×10⁵ RCAS-derived H3G34R (RCAS/H3G34R) tumor cells, expressing firefly luciferase, resulted in the robust growth of intracranial tumors after 28 days (FIGS. 10C and D).

2. Treatment with Tumor Lysate-Pulsed Dendritic Cells and Anti-PD-1 Monoclonal Antibodies Provides a Survival Benefit

The inventors next asked whether DC vaccination together with anti-PD-1 checkpoint blockade would provide a survival benefit in this preclinical model. RCAS/H3G34R tumor cells were implanted via intracranial injection, and after 7 days animals showing evidence of tumor take by chemiluminescence were injected intradermally with 10⁶ bone marrow-derived DCs pulsed with tumor lysate followed by an intraperitoneal injection of PD-1 monoclonal antibody (mAb). Control animals received an IgG1 isotype mAb without DC vaccination. PD-1 mAb and isotype mAb were administered again 48 hours later. The treatment group received a second round of DC vaccination and PD-1 mAb after 14 days (FIG. 11A). As shown in FIG. 11B several DC vaccinated animals survived longer than any of the animals in the control group. All of the surviving treated mice were sacrificed at day 83.

3. Identification of Candidate Tumor-Specific Antigens from Patient DHGs

Having established that DC vaccination with tumor lysate was a feasible treatment modality in the DHG model, the inventors next investigated whether a TSA-targeted peptide-pulsed DC (ppDC) vaccine would also provide a survival benefit. To identify potential peptide candidates, the inventors sequenced RNA extracted from two primary DHG tumors and implemented the IRIS bioinformatics pipeline. Since both patients were HLA-A*02:01-positive, the inventors focused on peptides that were predicted to bind with high affinity to this HLA class I allele. The initial screens yielded 99 candidate nonapeptides, which the inventors then manually curated. The inventors verified whether peptides resulted from exon inclusion or exon skipping, and filtered them through several other predictors of HLA binding and immunogenicity (Suppl. Table S2). The inventors found that three of the peptides were conserved in the mouse and also predicted to bind to the MHC class I allele H-2K^{b} that is carried by C57BL6/J mice (Table 1). The inventors employed a T2 cell HLA stabilization assay, with the K27M peptide identified by Okada's group as a positive control, to verify that the three peptides bound HLA-A*02:01 (FIG. 4), and also confirmed that the three genes from which the peptide sequences were derived were expressed in implanted RCAS/H3G34R tumors (Table 1).

4. Increased Number of CD8⁺ T Cells in Tumors Treated with Peptide-Pulsed or Tumor Lysate-Pulsed Dendritic Cells

The inventors have previously shown that the survival benefit of DC vaccination with concurrent PD-1 blockade is completely dependent upon CD8 T cell. To determine whether ppDC vaccination plus checkpoint inhibitor resulted in increased T cells in implanted RCAS/H3G34R tumors the inventors repeated the first in vivo experiment and sacrificed tumor-bearing animals on day 7 after the second injection of DCs (28 days after tumor implantation). The inventors added a third group of animals that were injected with DCs pulsed with one of the three peptides selected from the IRIS screen. Brains were either dissociated to obtain an immune cell-enriched fraction or fixed for immunohistochemistry. As shown in FIG. 13, the proportion of CD8⁺ T cells in CD45⁺ CD3⁺ cells in tumor infiltrating leukocytes (TILs) from animals treated with DCs pulsed with either lysate or TSAs was markedly higher, 43.8% and 50.5% respectively, compared with 3.12% with untreated tumor. CD3⁻ CD11b⁺ myeloid cells were predominantly Ly6C⁺ indicating that they were likely derived from circulating monocytes (FIG. 12).

Quantification of CD4, CD8, and CD11b immunostaining showed a higher density of T cells and myeloid cells in the tumor of a TSA ppDC-treated animal compared with an untreated animal (FIG. 13). The extent of neovascularization as visualized with a CD31 antibody (PECAM-1) appeared to be approximately the same; T cells and myeloid were evident in close proximity to these blood vessels, suggesting that they entered the tumor by diapedesis (FIG. 13). These observations are consistent with a CTL response caused by peripheral delivery of a TSA via targeted ppDC vaccination.

5. Treatment with Tripeptide-Pulsed Dendritic Cells and Anti-PD-1 Checkpoint Inhibitor Provides a Survival Benefit

To determine whether ppDC treatment would provide a survival benefit, the inventors injected tumor-bearing animals with DCs pulsed with three other TSAs identified in the IRIS screen (designated SYY). Animals in the control group received DCs pulsed with three H3.3 decapeptides that span glycine 34 (wild-type, G34R, and G34V sequences; designated NC). None of these peptides were predicted to bind mouse MHC class I alleles (Table 1). Both groups received anti-PD-1 mAb as previously described (FIG. 11A). Tumor growth was monitored by bioluminescent imaging over the first 28 days post-implantation, and animal survival was recorded over 48 days. Five animals from the SYY ppDC group were still alive after six months. As shown in FIG. 14, vaccination with SYY ppDCs slowed tumor growth and decreased mortality compared with the control group. During the course of the experiment, the inventors did not observe any immune pathology, which would be indicative of an autoimmune response, such as vitiligo.

The inventors sacrificed two animals from each group seven days after the second DC injection, and prepared leukocytes from brain, lymph nodes and spleen from four animals, two from each group. Profiling these cells by staining with an expanded panel of antibodies showed that T cells in TILs from the two animals treated with SYY ppDCs comprised 43.2% CD8⁺ and 44.5% CD4⁺ T cells of which 69% and 45.3% respectively expressed PD-1 (FIG. 6A). In contrast, T cells in the TILs from the two animals in the control group comprised fewer CD8⁺ T cells of which only 26.3% expressed PD-1 (FIG. 6C). There was about the same percentage of CD4⁺ T cells between the two groups however there were more double negative T cells in the control group.

The inventors do not know whether these latter T cells were originally CD4+, although the inventors suspect that they were because analysis of TILs from untreated tumor-bearing animals showed that the majority of the CD3⁺ TILs expressed CD4 (FIG. 12). The inventors found that only a small fraction of the CD4⁺ T cells expressed CD25 (6.4% in the NC ppDC-treated TILs, and 16.9% in the SYY ppDC-treated TILs), and the majority of these cells were also PD-1⁺ in the SYY ppDC-treated TILs (FIG. 8). FoxP3 was not in the antibody panel, thus the inventors do not know how many of the CD25⁺ CD4⁺ T cells were likely to be Tregs. From the total number of TILs isolated, the inventors estimate that treatment with the therapeutic peptide vaccine resulted in approximately six times as many infiltrating T cells (Suppl. Table S3).

Staining TILs with a myeloid cell antibody panel identified four different populations of CD3⁻ CD11b⁺ cells (FIG. 6): Ly6C⁺ CD11c⁺ F4/80⁺ XCR1⁺, Ly6C⁺ CD11c⁺ F4/80⁺ XCR1⁻, Ly6C⁻ CD11c⁺ F4/80⁺ XCR1⁻, Ly6C⁻ CD11c⁺ F4/80⁻ XCR1⁻. There was a relative increase in the percentage of CD11b⁺ Ly6C⁺ CD11c⁺ XCR1⁺ myeloid cells in the TILs from SYY ppDC-treated mice compared with TILs from NC ppDC-treated mice. Since XCR1 is a marker found on cross presenting conventional type 1 DCs, the inventors infer that these cells were likely to be monocyte-derived DCs (moDCs) or macrophages that can cross-present MHC class I-specific peptides to CD8⁺ T cells. The Ly6C-CD11c⁺ myeloid cells that did not express F4/80 were likely to be conventional type 2 DCs (FIG. 9). Based on the total TIL counts, the inventors estimate that there were about three times as many of myeloid cells in the TILs from animals treated with the SYY ppDCs (Suppl. Table S3).

### C. Discussion

Although it has been reported that oncohistone-driven HGGs in the HERBY Phase II trial were immunologically cold, a peptide that spans the H3K27M mutation was recently shown to elicit an immune response in patients with diffuse midline glioma, H3 K27-altered. In this report, the inventors provide preclinical support for treating DHG with DCs pulsed with identified TSAs combined with PD-1 mAb. In order to test this therapeutic modality, the inventors developed a syngeneic H3G34R murine tumor model using the RCAS/tv-A system. Transcriptome analysis of three independently derived non-clonal RCAS/H3G34R lines showed that they expressed TFs that maintain neural stem cells in a proliferative uncommitted state, including SOX2, HES1, HES5, FOXG1, and OLIG2. OLIG2 expression persists in the oligodendrocyte lineage, but the phosphorylation state of OLIG2 determines whether it promotes the proliferation of neural progenitors or oligodendrocyte differentiation. It has been reported that OLIG2 is not expressed in DHG, as a result of DNA methylation-induced transcriptional silencing. However, HGG characterized by PDGFRA amplification do express OLIG2, suggesting that the expression of OLIG2 in the mouse model, and possibly OLIG1 and SOX10, are a consequence of the forced overexpression of PDGF-B. Recent reports provide evidence that the cell of origin of DHG tumors may involve a ventral forebrain interneuron progenitor. The RCAS/H3G34R cell lines that the inventors generated also express proneural TFs that are required for the formation of interneurons, namely ASCL1 and GSX1. The cells expressed much less GSX2 (Suppl. Table S2), a second closely related homeodomain TF that is also required for interneuron differentiation. Based on the close proximity of GSX2 to PDFGRA on chromosome 4 recent evidence has been presented to explain how aberrant expression of PDGFRA may occur in an interneuron progenitor leading to oncogenesis. Single-cell RNA sequencing will resolve whether the same cells express TFs for determining both interneuron and oligodendrocyte lineages in the RCAS/H3G34R model.

The inventors found a statistically significant increase in survival after treatment with DCs pulsed with the three TSAs in combination with anti-PD-1 checkpoint inhibitor. The failure of PD-1 blockade combined with DCs pulsed with negative control peptides not predicted to bind H2-K^{b} provides evidence that the therapeutic effect that the inventors observed was directly attributable to TSA peptide selection. Profiling isolated TILs from tumor-bearing animals showed that there were more T cells and monocytes in the TME after exposure to the candidate therapeutic peptides compared with peptides that were not predicted to bind mouse MHC class I alleles even though both groups received a checkpoint inhibitor. This observation indicates that animals treated with the therapeutic peptides mounted a more efficacious cellular immune response which led to an increase in the number of TILs, and a positive therapeutic outcome. In summary, in a preclinical model, the inventors have demonstrated the immunotherapeutic benefit of DC vaccination targeting TSAs derived from AS events that are enriched in the transcriptome of DHG. In future directions, they will identify TSAs conserved among patient DHGs, as well as test whether synthetic long peptides encompassing these antigens or RNA-based vaccination methods will alter therapeutic efficacy or feasibility.

### D. Materials and Methods

### 1. Derivation of an H3G34R RCAS/Tv-a Model

All of the animal experiments were approved by the UCLA Institutional Animal Care and Use Committee. Surgical procedures were carried out in a BSL2 biological safety cabinet. H3 G34R HGG were generated essentially as described by Misuraca et al., Postnatal day 2-3 pups were anesthetized with a mixture isofluorane/oxygen delivered through a nose cone trimmed to fit the neonate. Two microliters of packages cells comprising equal numbers of DF1 cells producing RCAS viruses encoding H3G34R, Cre recombinasc, and PDGF-B in phosphate-buffered saline (PBS) were injected (1×10⁵ cells per μl) into the left lateral ventricle through a 28-gauge needle attached to a 10 μl Hamilton syringe. The needle penetrated the skull to a depth of ˜2 mm at a location ˜0.25 mm lateral to the sagittal suture and 0.5-0.75 mm rostral to the neonatal coronary suture. Animals were returned to their home cages and monitored daily after weaning for signs of tumor formation as evidenced by loss of body weight and problems with locomotion at which point the animal was euthanized with CO₂, and the brain removed. A Brain Tumor Dissociation Kit (P) (Miltenyi Biotec Inc., Auburn, CA) was used to dissociate the mouse brains, which were then fractionated on a 70%/30% Percoll® gradient (Sigma-Aldrich, St. Louis, MO) to the remove myelin. Cells were placed in culture in medium formulated to support the growth of human pediatric HGG. All injected animals were sacrificed after eighteen weeks.

### 2. Orthotopic Delivery of Tumor Cells and Treatment Protocol

Under ketamine/xylazine anesthesia, a burr hole was drilled in the skull 2 mm lateral to Bregma and 0.5 mm caudal and injections of 1×10⁵ RCAS/H3G34R tumor cells in 2 μL of PBS were made into the left hemispheres of eight-week-old syngeneic C57Bl/6J female mice. Cells were injected through a 28-gauge needle attached to a 10 μl Hamilton syringe at a depth of 2.5 mm. DCs were prepared from bone marrow (BM) essentially as previously described. In brief, BM cells were plated in RPMI-1640 plus 10% fetal bovine serum (FBS) with antibiotics, and after 24 hours the nonadherent cells were counted and re-plated in 12-well tissue culture plates (˜2×10⁶ cells per well) in RPMI-1640 plus 10% FBS supplemented with murine granulocyte-macrophage colony stimulating factor (GM-CSF, 100 ng/ml; Biolegend, San Diego, CA) and interleukin-4 (IL-4, 500 U/ml; Biolegend). The medium was replaced after three days, and three days later the adherent differentiated DCs were pulsed overnight with 250 μg per well of a freeze thawed lysate of RCAS/H3G34R cells or 10 μg of each peptide per well. Cells were harvested, washed, resuspended in PBS and 1×10⁶ cells were injected intradermally seven days after tumor implantation together with an IP injection of anti-mouse PD-1 (250 μg per animal; clone RMP1-14; Bio X Cell, Lebanon, NH). PD-1 mAb was re-administered 48 hours later. The same treatment regimen was repeated 21 days after tumor implantation. In vivo imaging was performed under isoflurane anesthesia after IP injection of luciferin (100 μl of 5 mg/ml stock solution). Chemiluminescence images were captured using an IVIS Lumina II imaging system (Perkin Elmer, Waltham, MA). Log transformed mean radiance values and standard errors, and survival data were analyzed and plotted in GraphPad Prism (GraphPad Software Corp., San Diego, CA). Graphs were exported as enhanced metafiles to CorelDraw2017 (Corel Corporation, Ottawa, Canada).

### 3. DNA Sequencing

Genomic DNA was isolated from cultured RCAS tumor cells using a commercially available kit (Quick-DNA miniprep kit, Zymo Research, Irvine CA). To confirm the presence of the G34R mutation, PCR primers flanking the cloning site in the RCAS vector were used to amplify inserted sequences (forward primer 5′ GTCTGTGTGCTGCAGGAGCTGAGCTGACTCTGCTG 3′ (SEQ ID NO:108), reverse primer 5′ GATACGCGTATATCTGGCCCGTACATCGCATCG 3′ (SEQ ID NO:109)), which were treated with ExoSAP-IT™ (ThermoFisher, Waltham, MA) then sequenced using an H3.3-specific primer (5′GCACGTTCTCCACGTATGCGGCGTG 3′ (SEQ ID NO:110)).

### 4. RNA Sequencing and IRIS Pipeline

Total RNA was extracted from human and mouse tumors and cells using commercially available kits either RNAeasy kit (Qiagen, Germantown, MD) or Direct-zol RNA kit (Zymo Research). The library constructed from the human tumors LB3570 and LB4179 was pair-end sequenced (100 bp). The mouse tumors were single-end sequenced (50 bp). Full description of the IRIS pipeline is found in Pan, et al. and the software is available on GitHub (https://github.com/Xinglab/IRIS). The IRIS pipeline combines in depth quantification of alternative splicing events in tumors by rMATS-turbo with a statistically robust screening workflow to identify alternative splicing events that are over-represented or exclusively expressed in the tumor transcriptome. This target screening workflow leverages a unified large-scale alternative splicing reference database representing normal and tumor splicing profiles derived from the TCGA and GTEx databases. The screening workflow is followed by the translation of splice junction peptide sequences and prediction of HLA-binding affinities. In this study, the LB3570 and LB4179 RNA-seq data were processed by IRIS's data processing module using STAR v2.5.3a ‘two-pass mode’ followed by rMATS-turbo v4.0.2. Then, target screening is performed through IRIS's ‘personalized mode’, which compares the tumor transcriptome to 1,409 samples of GTEx normal brain tissues from the IRIS reference database and identifies tumor-associated events based on the Tukey's rule and a threshold of splicing level (Percent-Spliced-In value) difference of >5%. The identified tumor-associated events were translated and subjected to HLA binding prediction algorithms. The primary HLA-binding prediction is based on the Immune Epitope Database (IEDB) ‘recommended’ mode.

### 5. Flow Cytometry

A leukocyte fraction was isolated from tumor-bearing animals by dissociating brain tissue as described above and fractionating on a 30%: 70% Percoll® gradient. Isolated lymph nodes and spleens were macerated in RPMI-1640 and strained through a 70-micron sieve. The following mAbs were used to label cells: CD45 (Alexa Fluor (AF) 700-conjugated; clone 30-F11), CD3 (Allophycocyanin (APC)-Cyanine 7 (Cy7)-conjugated; clone 145-2C11), CD4 (APC-conjugated; clone RM4-5), CD8 (Phycocrythrin (PE)-Cy7-conjugated; clone 53-6.7), CD25 (Brilliant Violet (BV) 650-conjugated; clone PC61), PD-1 (PE-conjugated; clone RMP1-14), CD45R/B220 (Peridinin chlorophyll protein (PerCP)-Cy5.5-conjugated; clone RA3-6B2), and NK1.1 (Fluorescein isothiocyanate (FITC)-conjugated; clone PK136), plus Zombie Violet™, CD11b (PE-conjugated; clone M1/70), CD11c (PerCP-Cy5.5 conjugated; clone N418), Ly6C (BV650-conjugated; clone Hk1.4), Ly6G (PE-Cy-conjugated; clone RB6-8C5), F4/80 (FITC-conjugated; clone BM8), and XCR1 (APC-conjugated; clone ZET) plus Zombie Violet™. All antibodies were purchased from Biolegend, and samples were run on a LSRII analytical flow cytometer (Becton Dickinson, Franklin Lakes, NJ). Individual FCS files were analyzed using FlowJo® software (FlowJo LLC, Ashland, OR); 2-D contour plots were exported as .svg files to CorelDraw2017.

### 6. T2 Cell HLA Stabilization Assay

HLA-A*02:01 TAP-deficient T2 cells (2×10⁵ in 200 μL) were incubated with peptides (25 μg per ml in 10% DMSO in PBS) in AIM-V plus 1% human serum at 25° C. overnight to facilitate peptide binding by reducing HLA turnover. The following day the cells were returned to 37° C. for 5 hours then stained with a pan HLA antibody (FITC-conjugated HLA, B, C; clone W6/32). HLA class I expression was measured by flow cytometry, and the percent increase in mean fluorescence intensity over cells incubated with 10% DMSO alone was calculated as follows:

$$\frac{mean fluorescence intensity {( MFI )}^{peptide treated cells} - {MFI}^{untreated cells}}{{MFI}^{untreated cells}} \times 100$$

### 7. Immunostaining

Whole brains were fixed by immersion in IHC Zinc Fixative (BD Biosciences, San Jose, CA), then embedded in paraffin. Sections (5 um) were stained using Opal Fluorophore reagents (Akoya Biosciences, Marlborough, MA) and a Leica Bond RX auto-stainer (Leica Biosystems, Vista, CA) following antigen retrieval. The following antibodies were used CD4 (Opal 570), CD8 (Opal480), CD11b (Opal 690) and CD31 (Opal 780). Images were collected using a Leica Aperio Versa 200 Slide Scanning Microscope equipped with a 16-bit 5.5-megapixel fluorescence camera. Cell densities were calculated using HALO image analysis software (Indica Labs, Albuquerque, NM). In brief, brain and tumor were distinguished based on the difference in density of the DAPI nuclear stain (tumor had higher cell density than the surrounding parenchyma), and a region of interest (ROI) was drawn. The threshold for positivity for each fluorophore was determined separately to minimize spillover between channels. Each cell type was defined by a fluorescent signal above threshold in only one channel that co-registered with a single DAPI⁺ nucleus. The densities of each cell type within the ROI were then calculated (cells per mm² tissue area).

### E. Tables

TABLE 1
Calculated affinities of peptides for
H-2K^{b} and expression of corresponding genes.

| | | SEQ ID | NetMHCpan | NetMHCpan | Gene |
|---|---|---|---|---|---|
| Gene | Peptide | NO | BA 4.1* | EL 4.1 | expression^{¶} |
| Arhgap4 | YLFTFLNHL | 39 | 34.1 | 0.7395 | 53.88 ± 1.22 |
| At12 | YMYNKVAVL | 33 | 59.1 | 0.5388 | 9.94 ± 0.62 |
| Sfxn5 | SMLEKTALL | 32 | 93.08 | 0.5033 | 56.93 ± 8.93 |
| H3-3a | KSAPSTGRVK | 11 | 24650.91 | 0.0316 | 12.22 ± 0.22 |
| | KSAPSTGVVK | 9 | 24867.54 | 0.0146 | |
| | KSAPSTGGVK | 35 | 24967.28 | 0.0114 | |

*nM
^{¶}TMM normalized counts (n = 3, mean ± SEM)

TABLE S2
Curated list of candidate peptides from IRIS screen of transcriptomes from G34R patient tumors

| | | | | | | | SEQ | | |
|---|---|---|---|---|---|---|---|---|---|
| NetMHCpan¹ | NetMHCpan² | | | | Deep- | | ID | | Alternative |
| BA 4.1 | EL 4.1 | HLAthena² | Prime³ | MHCflurry² | Immuno⁴ | Peptide | NO | Gene | splicing |
| 1.78 | 0.9939 | 0.9982 | 0.014 | 0.9926 | 0.6507 | FIF/EHSYSV | 46 | THAP6 | exon skip |
| 1.98 | 0.9870 | 0.9993 | 0.033 | 0.9570 | 0.6849 | SLMDKLL/PV | 86 | FAM149B1 | exon skip |
| 2.13 | 0.9784 | 0.9995 | 0.015 | 0.9796 | 0.7413 | FLSDLNL/LV | 57 | QARS | exon skip |
| 2.32 | 0.9855 | 0.9989 | 0.007 | 0.9924 | 0.6610 | FLSDTQ/VFV | 58 | MIOS | exon skip |
| 2.44 | 0.9869 | 0.9997 | 0.007 | 0.9948 | 0.5735 | YLFNS/VVNV | 98 | YPEL4 | exon skip |
| 2.56 | 0.8296 | 0.9780 | 0.099 | 0.9034 | 0.6928 | TMWDYT/IPI | 87 | CHPT1 | exon skip |
| 3.1 | 0.9723 | 0.9996 | 0.025 | 0.9777 | 0.7973 | FLLDLDPLL/ | 54 | ALDH5A1 | exon |
| | | | | | | | | | inclusion |
| 3.14 | 0.9781 | 0.9993 | 0.063 | 0.9945 | 0.7470 | FLFERVEGI | 50 | HDAC11 | exon |
| | | | | | | | | | inclusion |
| 4.13 | 0.9828 | 0.9986 | 0.013 | 0.9928 | 0.8381 | M/LADIPVTI | 79 | HERC4 | exon skip |
| 4.15 | 0.9366 | 0.9920 | 0.011 | 0.9935 | 0.7329 | YLFTFLNH/L | 39 | ARHGAP4 | exon |
| | | | | | | | | | inclusion |
| 4.15 | 0.8575 | 0.9758 | 0.019 | 0.9890 | 0.8055 | LMN/GLIMTV | 78 | CACHD1 | exon skip |
| 4.39 | 0.9815 | 0.9945 | 0.004 | 0.9819 | 0.5912 | YLD/GIITIV | 97 | CBWD3 | exon |
| | | | | | | | | | inclusion |
| 4.86 | 0.9772 | 0.9874 | 0.043 | 0.9849 | 0.7060 | GLWEEAYR/L | 65 | IFT172 | exon skip |
| 5.34 | 0.9375 | 0.9961 | 0.01 | 0.9842 | 0.8277 | KMLDKLRY/V | 72 | DTNB | exon |
| | | | | | | | | | inclusion |
| 5.48 | 0.9810 | 0.9994 | 0.01 | 0.9914 | 0.9509 | FLG/PVIVEI | 52 | ANK3 | exon |
| | | | | | | | | | inclusion |
| 6.63 | 0.9413 | 0.9889 | 0.024 | 0.9906 | 0.6782 | NL/LAEIHGV | 83 | DPYSL4 | exon skip |
| 7.02 | 0.9687 | 0.9995 | 0.015 | 0.9556 | 0.8524 | ALLDE/VLDV | 39 | NELFCD | exon skip |
| 7.31 | 0.9532 | 0.9894 | 0.049 | 0.9878 | 0.7303 | ALFGDVKFV/ | 38 | UPP1 | exon |
| | | | | | | | | | inclusion |
| 7.47 | 0.9046 | 0.9724 | 0.023 | 0.9556 | 0.5118 | VQWDLLH/GV | 91 | MPDU1 | exon skip |
| 7.62 | 0.9498 | 0.9971 | 0.009 | 0.9855 | 0.5477 | VMDNLLIQV | 90 | TYW5 | exon |
| | | | | | | | | | inclusion |
| 8.28 | 0.9431 | 0.9979 | 0.026 | 0.9917 | 0.8604 | VLWN/GIPTA | 88 | PCSK7 | exon |
| | | | | | | | | | inclusion |
| 10.71 | 0.9180 | 0.8958 | 0.006 | 0.9711 | 0.6640 | FLA/QKCHTL | 5 | HAUS2 | exon skip |
| 11.29 | 0.9240 | 0.9957 | 0.014 | 0.9752 | 0.5355 | AMYSVEITV | 44 | PALM | exon |
| | | | | | | | | | inclusion |
| 13.43 | 0.8330 | 0.9482 | 0.033 | 0.9565 | 0.5762 | VLYTIFMK/V | 89 | LETMD1 | exon skip |
| 13.46 | 0.8884 | 0.8685 | 0.041 | 0.9740 | 0.5364 | KMYKT/PIFL | 74 | ITSN2 | exon |
| | | | | | | | | | inclusion |
| 14.56 | 0.8857 | 0.9549 | 0.047 | 0.9734 | 0.6828 | SMLEK/TALL | 32 | SFXN5 | exon |
| | | | | | | | | | inclusion |
| 5.6 | 0.9035 | 0.9890 | 0.109 | 0.9749 | 0.7456 | YMYNK/VAVL | 33 | ATL2 | exon skip |
| 48.4 | 0.9274 | 0.9988 | 0.011 | 0.9909 | 0.6441 | /TLSQAIVKV | 1 | U2SURP | exon |
| | | | | | | | | | inclusion |

¹<50 nM predicts good binding, ² high score predicts good binding, ³low percent ranking predicts good binding, ⁴high score predicts good immunogenicity. The position of the splice junction is marked by a red slash mark; three of the peptides do not span two exons, but are located within the included exon. Peptides in bold are predicted to bind to mouse H-2K^{b}, however the peptide sequence from LETMD1 is not conserved in the mouse gene.

TABLE S3
Estimated total number of cells in CD45+ subsets.

| | Lymph nodes | | | Spleen | | | TILs | | |
|---|---|---|---|---|---|---|---|---|---|
| | SYY | G34 | ratio | SYY | G34 | ratio | SYY | G34 | ratio |
| Lymphoid | | | | | | | | | |
| cell panel | | | | | | | | | |
| Total CD45+ | 1.5 × 10⁷ | 1.49 × 10⁷ | 1.0 | 5.19 × 10⁷ | 4.93 × 10⁷ | 1.05 | 1.63 × 10⁶ | 4.57 × 10⁵ | 3.57 |
| CD3+ | 1.38 × 10⁷ | 1.32 × 10⁷ | 1.04 | 4.17 × 10⁷ | 3.66 × 10⁷ | 1.14 | 5.61 × 10⁵ | 8.97 × 10⁴ | 6.25 |
| CD8+ | 6.57 × 10⁶ | 6.33 × 10⁶ | 1.04 | 2.04 × 10⁷ | 1.43 × 10⁷ | 1.43 | 2.42 × 10⁵ | 1.78 × 10⁴ | 13.6 |
| CD8+ PD-1+ | 0 | 0 | | 0 | 0 | | 1.62 × 10⁵ | 4.78 × 10³ | 33.89 |
| CD4+ | 6.98 × 10⁶ | 6.73 × 10⁶ | 1.04 | 1.99 × 10⁷ | 2.09 × 10⁷ | 0.95 | 2.5 × 10⁵ | 4.46 × 10⁴ | 5.6 |
| CD4+ CD25+ | 1.63 × 10⁵ | 1.63 × 10⁵ | 1.0 | 4.78 × 10⁵ | 4.09 × 10⁵ | 1.17 | 1.13 × 10⁵ | 9.05 × 10³ | 12.49 |
| CD4+ PD-1+ | 0 | 0 | | 0 | 0 | | 4.55 × 10⁴ | 3.29 × 10³ | 13.83 |
| CD8− CD4− | 2.04 × 10⁵ | 1.45 × 10⁵ | 1.41 | 4.59 × 10⁵ | 1.20 × 10⁵ | 3.8 | 6.79 × 10⁴ | 2.59 × 10⁴ | 2.62 |
| CD3− | 1.95 × 10⁶ | 5.93 × 10⁵ | 3.29 | 2.06 × 10⁶ | 1.23 × 10⁷ | 1.67 | 1.02 × 10⁶ | 3.62 × 10⁵ | 2.82 |
| B cells | 1.50 × 10⁶ | 4.29 × 10⁵ | 3.5 | 1.49 × 10⁶ | 9.33 × 10⁶ | 0.16 | 9.30 × 10⁴ | 1.08 × 10⁴ | 8.61 |
| NK cells | 2.00 × 10⁵ | 8.24 × 10⁴ | 2.43 | 2.87 × 10⁵ | 2.03 × 10⁶ | 0.14 | 3.55 × 10⁵ | 1.17 × 10⁵ | 3.03 |
| Myeloid | | | | | | | | | |
| cell panel | | | | | | | | | |
| Total CD45+ | 1.45 × 10⁷ | 1.56 × 10⁷ | 0.93 | 4.83 × 10⁷ | 5.10 × 10⁷ | 0.95 | 1.74 × 10⁶ | 4.41 × 10⁵ | 3.95 |
| CD3− CD11b+ | 2.76 × 10⁴ | 2.96 × 10⁴ | 0.93 | 3.96 × 10⁶ | 1.6 × 10⁶ | 2.48 | 5.68 × 10⁵ | 1.92 × 10⁵ | 2.96 |
| Ly6C+ Ly6G− | 8.11 × 10³ | 8.43 × 10³ | 0.96 | 1.69 × 10⁶ | 7.43 × 10⁵ | 2.27 | 4.52 × 10⁵ | 1.61 × 10⁵ | 2.81 |
| CD11c+ XCR1+ | 1.01 × 10² | 2.06 × 10² | 0.53 | 2.18 × 10⁴ | 2.97 × 10⁴ | 0.73 | 2.54 × 10⁵ | 8.41 × 10⁴ | 3.02 |
| CD11c+ XCR1− | 7.09 × 10³ | 8.23 × 10³ | 0.86 | 1.66 × 10⁶ | 7.01 × 10⁵ | 2.37 | 1.94 × 10⁵ | 7.53 × 10⁴ | 2.58 |
| Ly6C− Ly6G− | 1.8 × 10⁴ | 1.97 × 10⁴ | 0.91 | 2.12 × 10⁶ | 8.04 × 10⁵ | 2.64 | 7.22 × 10⁴ | 1.40 × 10⁴ | 5.16 |
| CD11c+ XCR1− | 4.31 × 10³ | 1.45 × 10³ | 2.96 | 1.38 × 10⁴ | 2.82 × 10⁴ | 0.49 | 3.32 × 10⁴ | 4.01 × 10³ | 8.27 |
| F4/80⁺ | | | | | | | | | |
| CD11c+ XCR1− | 1.37 × 10⁴ | 1.83 × 10⁴ | 0.75 | 2.11 × 10⁶ | 7.76 × 10⁵ | 2.72 | 3.61 × 10⁴ | 9.15 × 10³ | 3.95 |
| F4/80⁻ | | | | | | | | | |

### REFERENCES

The following references and the publications referred to throughout the specification, to the extent that they provide exemplary procedural or other details supplementary to those set forth herein, are specifically incorporated herein by reference.
1. S. Deorah, C. F. Lynch, Z. A. Sibenaller, T. C. Ryken, Trends in brain cancer incidence and survival in the United States: Surveillance, Epidemiology, and End Results Program, 1973 to 2001. Neurosurg Focus 20, E1 (2006).
2. R. Stupp, W. P. Mason, M. J. van den Bent, M. Weller, B. Fisher, M. J. Taphoorn, K. Belanger, A. A. Brandes, C. Marosi, U. Bogdahn, J. Curschmann, R. C. Janzer, S. K. Ludwin, T. Gorlia, A. Allgeier, D. Lacombe, J. G. Cairncross, E. Eisenhauer, R. O. Mirimanoff, R. European Organisation for, T. Treatment of Cancer Brain, G. Radiotherapy, G. National Cancer Institute of Canada Clinical Trials, Radiotherapy plus concomitant and adjuvant temozolomide for glioblastoma. N Engl J Med 352, 987-996 (2005).
3. A. Sottoriva, I. Spiteri, S. G. Piccirillo, A. Touloumis, V. P. Collins, J. C. Marioni, C. Curtis, C. Watts, S. Tavare, Intratumor heterogeneity in human glioblastoma reflects cancer evolutionary dynamics. Proc Natl Acad Sci USA 110, 4009-4014 (2013).
4. J. Wang, E. Cazzato, E. Ladewig, V. Frattini, D. I. Rosenbloom, S. Zairis, F. Abate, Z. Liu, O. Elliott, Y. J. Shin, J. K. Lee, I. H. Lee, W. Y. Park, M. Eoli, A. J. Blumberg, A. Lasorella, D. H. Nam, G. Finocchiaro, A. Iavarone, R. Rabadan, Clonal evolution of glioblastoma under therapy. Nature genetics 48, 768-776 (2016).
5. I. Tirosh, A. S. Venteicher, C. Hebert, L. E. Escalante, A. P. Patel, K. Yizhak, J. M. Fisher, C. Rodman, C. Mount, M. G. Filbin, C. Neftel, N. Desai, J. Nyman, B. Izar, C. C. Luo, J. M. Francis, A. A. Patel, M. L. Onozato, N. Riggi, K. J. Livak, D. Gennert, R. Satija, B. V. Nahed, W. T. Curry, R. L. Martuza, R. Mylvaganam, A. J. Iafrate, M. P. Frosch, T. R. Golub, M. N. Rivera, G. Getz, O. Rozenblatt-Rosen, D. P. Cahill, M. Monje, B. E. Bernstein, D. N. Louis, A. Regev, M. L. Suva, Single-cell RNA-seq supports a developmental hierarchy in human oligodendroglioma. Nature 539, 309-313 (2016).
6. A. S. Venteicher, I. Tirosh, C. Hebert, K. Yizhak, C. Neftel, M. G. Filbin, V. Hovestadt, L. E. Escalante, M. L. Shaw, C. Rodman, S. M. Gillespie, D. Dionne, C. C. Luo, H. Ravichandran, R. Mylvaganam, C. Mount, M. L. Onozato, B. V. Nahed, H. Wakimoto, W. T. Curry, A. J. Iafrate, M. N. Rivera, M. P. Frosch, T. R. Golub, P. K. Brastianos, G. Getz, A. P. Patel, M. Monje, D. P. Cahill, O. Rozenblatt-Rosen, D. N. Louis, B. E. Bernstein, A. Regev, M. L. Suva, Decoupling genetics, lineages, and microenvironment in IDH-mutant gliomas by single-cell RNA-seq. Science 355, (2017).
7. A. P. Patel, I. Tirosh, J. J. Trombetta, A. K. Shalek, S. M. Gillespie, H. Wakimoto, D. P. Cahill, B. V. Nahed, W. T. Curry, R. L. Martuza, D. N. Louis, O. Rozenblatt-Rosen, M. L. Suva, A. Regev, B. E. Bernstein, Single-cell RNA-seq highlights intratumoral heterogeneity in primary glioblastoma. Science 344, 1396-1401 (2014).
8. J. Schwartzentruber, A. Korshunov, X. Y. Liu, D. T. Jones, E. Pfaff, K. Jacob, D. Sturm, A. M. Fontebasso, D. A. Quang, M. Tonjes, V. Hovestadt, S. Albrecht, M. Kool, A. Nantel, C. Konermann, A. Lindroth, N. Jager, T. Rausch, M. Ryzhova, J. O. Korbel, T. Hielscher, P. Hauser, M. Garami, A. Klekner, L. Bognar, M. Ebinger, M. U. Schuhmann, W. Scheurlen, A. Pekrun, M. C. Fruhwald, W. Roggendorf, C. Kramm, M. Durken, J. Atkinson, P. Lepage, A. Montpetit, M. Zakrzewska, K. Zakrzewski, P. P. Liberski, Z. Dong, P. Siegel, A. E. Kulozik, M. Zapatka, A. Guha, D. Malkin, J. Felsberg, G. Reifenberger, A. von Deimling, K. Ichimura, V. P. Collins, H. Witt, T. Milde, O. Witt, C. Zhang, P. Castelo-Branco, P. Lichter, D. Faury, U. Tabori, C. Plass, J. Majewski, S. M. Pfister, N. Jabado, Driver mutations in histone H3.3 and chromatin remodelling genes in paediatric glioblastoma. Nature 482, 226-231 (2012).
9. G. Wu, A. Broniscer, T. A. McEachron, C. Lu, B. S. Paugh, J. Becksfort, C. Qu, L. Ding, R. Hucther, M. Parker, J. Zhang, A. Gajjar, M. A. Dyer, C. G. Mullighan, R. J. Gilbertson, E. R. Mardis, R. K. Wilson, J. R. Downing, D. W. Ellison, J. Zhang, S. J. Baker, P. St. Jude Children's Research Hospital-Washington University Pediatric Cancer Genome, Somatic histone H3 alterations in pediatric diffuse intrinsic pontine gliomas and non-brainstem glioblastomas. Nat Genet 44, 251-253 (2012).

10. L. Bjerke, A. Mackay, M. Nandhabalan, A. Burford, A. Jury, S. Popov, D. A. Bax, D. Carvalho, K. R. Taylor, M. Vinci, I. Bajrami, I. M. McGonnell, C. J. Lord, R. M. Reis, D. Hargrave, A. Ashworth, P. Workman, C. Jones, Histone H3.3. mutations drive pediatric glioblastoma through upregulation of MYCN. Cancer Discov 3, 512-519 (2013).
11. P. W. Lewis, M. M. Muller, M. S. Koletsky, F. Cordero, S. Lin, L. A. Banaszynski, B. A. Garcia, T. W. Muir, O. J. Becher, C. D. Allis, Inhibition of PRC2 activity by a gain-of-function H3 mutation found in pediatric glioblastoma. Science 340, 857-861 (2013).
12. S. U. Jain, S. Khazaci, D. M. Marchione, S. M. Lundgren, X. Wang, D. N. Weinberg, S. Deshmukh, N. Juretic, C. Lu, C. D. Allis, B. A. Garcia, N. Jabado, P. W. Lewis, Histone H3.3 G34 mutations promote aberrant PRC2 activity and drive tumor progression. Proc Natl Acad Sci USA 117, 27354-27364 (2020).
13. G. L. Brien, R. B. Bressan, C. Monger, D. Gannon, E. Lagan, A. M. Doherty, E. Healy, H. Neikes, D. J. Fitzpatrick, O. Deevy, V. Grant, M. A. Marques-Torrejon, N. Alfazema, S. M. Pollard, A. P. Bracken, Simultaneous disruption of PRC2 and enhancer function underlies histone H3.3-K27M oncogenic activity in human hindbrain neural stem cells. Nat Genet 53, 1221-1232 (2021).
14. D. Sturm, H. Witt, V. Hovestadt, D. A. Khuong-Quang, D. T. Jones, C. Konermann, E. Pfaff, M. Tonjes, M. Sill, S. Bender, M. Kool, M. Zapatka, N. Becker, M. Zucknick, T. Hielscher, X. Y. Liu, A. M. Fontebasso, M. Ryzhova, S. Albrecht, K. Jacob, M. Wolter, M. Ebinger, M. U. Schuhmann, T. van Meter, M. C. Fruhwald, H. Hauch, A. Pekrun, B. Radlwimmer, T. Nichues, G. von Komorowski, M. Durken, A. E. Kulozik, J. Madden, A. Donson, N. K. Foreman, R. Drissi, M. Fouladi, W. Scheurlen, A. von Deimling, C. Monoranu, W. Roggendorf, C. Herold-Mende, A. Unterberg, C. M. Kramm, J. Felsberg, C. Hartmann, B. Wiestler, W. Wick, T. Milde, O. Witt, A. M. Lindroth, J. Schwartzentruber, D. Faury, A. Fleming, M. Zakrzewska, P. P. Liberski, K. Zakrzewski, P. Hauser, M. Garami, A. Klekner, L. Bognar, S. Morrissy, F. Cavalli, M. D. Taylor, P. van Sluis, J. Koster, R. Versteeg, R. Volckmann, T. Mikkelsen, K. Aldape, G. Reifenberger, V. P. Collins, J. Majewski, A. Korshunov, P. Lichter, C. Plass, N. Jabado, S. M. Pfister, Hotspot mutations in H3F3A and IDHI define distinct epigenetic and biological subgroups of glioblastoma. Cancer Cell 22, 425-437 (2012).
15. S. Nagaraja, M. A. Quezada, S. M. Gillespie, M. Arzt, J. J. Lennon, P. J. Woo, V. Hovestadt, M. Kambhampati, M. G. Filbin, M. L. Suva, J. Nazarian, M. Monje, Histone Variant and Cell Context Determine H3K27M Reprogramming of the Enhancer Landscape and Oncogenic State. Mol Cell 76, 965-980 e912 (2019).
16. R. B. Bressan, B. Southgate, K. M. Ferguson, C. Blin, V. Grant, N. Alfazema, J. C. Wills, M. A. Marques-Torrejon, G. M. Morrison, J. Ashmore, F. Robertson, C. A. C. Williams, L. Bradley, A. von Kriegsheim, R. A. Anderson, S. R. Tomlinson, S. M. Pollard, Regional identity of human neural stem cells determines oncogenic responses to histone H3.3 mutants. Cell Stem Cell 28, 877-893 e879 (2021).
17. K. Funato, R. C. Smith, Y. Saito, V. Tabar, Dissecting the impact of regional identity and the oncogenic role of human-specific NOTCH2NL in an hESC model of H3.3G34R-mutant glioma. Cell Stem Cell 28, 894-905 e897 (2021).
18. S. Haase, F. M. Nunez, J. C. Gauss, S. Thompson, E. Brumley, P. Lowenstein, M. G. Castro, Hemispherical Pediatric High-Grade Glioma: Molecular Basis and Therapeutic Opportunities. Int J Mol Sci 21, (2020).
19. A. Mackay, A. Burford, D. Carvalho, E. Izquierdo, J. Fazal-Salom, K. R. Taylor, L. Bjerke, M. Clarke, M. Vinci, M. Nandhabalan, S. Temelso, S. Popov, V. Molinari, P. Raman, A. J. Waanders, H. J. Han, S. Gupta, L. Marshall, S. Zacharoulis, S. Vaidya, H. C. Mandeville, L. R. Bridges, A. J. Martin, S. Al-Sarraj, C. Chandler, H. K. Ng, X. Li, K. Mu, S. Trabelsi, D. H. Brahim, A. N. Kisljakov, D. M. Konovalov, A. S. Moore, A. M. Carcaboso, M. Sunol, C. de Torres, O. Cruz, J. Mora, L. I. Shats, J. N. Stavale, L. T. Bidinotto, R. M. Reis, N. Entz-Werle, M. Farrell, J. Cryan, D. Crimmins, J. Caird, J. Pears, M. Monje, M. A. Debily, D. Castel, J. Grill, C. Hawkins, H. Nikbakht, N. Jabado, S. J. Baker, S. M. Pfister, D. T. W. Jones, M. Fouladi, A. O. von Bueren, M. Baudis, A. Resnick, C. Jones, Integrated Molecular Meta-Analysis of 1,000 Pediatric High-Grade and Diffuse Intrinsic Pontine Glioma. Cancer Cell 32, 520-537 e525 (2017).
20. D. Hambardzumyan, N. M. Amankulor, K. Y. Helmy, O. J. Becher, E. C. Holland, Modeling Adult Gliomas Using RCAS/t-va Technology. Transl Oncol 2, 89-95 (2009).
21. K. L. Misuraca, K. L. Barton, A. Chung, A. K. Diaz, S. J. Conway, D. L. Corcoran, S. J. Baker, O. J. Becher, Pax3 expression enhances PDGF-B-induced brainstem gliomagenesis and characterizes a subset of brainstem glioma. Acta Neuropathol Commun 2, 134 (2014).
22. M. O. Gratton, E. Torban, S. B. Jasmin, F. M. Theriault, M. S. German, S. Stifani, Hes6 promotes cortical neurogenesis and inhibits Hes1 transcription repression activity by multiple mechanisms. Mol Cell Biol 23, 6922-6935 (2003).
23. C. Hanashima, S. C. Li, L. Shen, E. Lai, G. Fishell, Foxg1 suppresses early cortical cell fate. Science 303, 56-59 (2004).
24. M. Bergsland, M. Werme, M. Malewicz, T. Perlmann, J. Muhr, The establishment of neuronal properties is controlled by Sox4 and Sox11. Genes Dev 20, 3475-3486 (2006).
25. M. Bergsland, D. Ramskold, C. Zaouter, S. Klum, R. Sandberg, J. Muhr, Sequentially acting Sox transcription factors in neural lineage development. Genes Dev 25, 2453-2464 (2011).
26. Y. Sun, D. H. Meijer, J. A. Alberta, S. Mehta, M. F. Kane, A. C. Tien, H. Fu, M. A. Petryniak, G. B. Potter, Z. Liu, J. F. Powers, I. S. Runquist, D. H. Rowitch, C. D. Stiles, Phosphorylation state of Olig2 regulates proliferation of neural progenitors. Neuron 69, 906-917 (2011).
27. I. Imayoshi, R. Kageyama, bHLH factors in self-renewal, multipotency, and fate choice of neural progenitor cells. Neuron 82, 9-23 (2014).
28. F. F. Vasconcelos, D. S. Castro, Transcriptional control of vertebrate neurogenesis by the proneural factor Ascl1. Front Cell Neurosci 8, 412 (2014).
29. M. Andreatta, M. Nielsen, Gapped sequence alignment using artificial neural networks: application to the MHC class I system. Bioinformatics 32, 511-517 (2016).
30. R. Elkington, S. Walker, T. Crough, M. Menzies, J. Tellam, M. Bharadwaj, R. Khanna, Ex vivo profiling of CD8+-T-cell responses to human cytomegalovirus reveals broad and multispecific reactivities in healthy virus carriers. J Virol 77, 5226-5240 (2003).
31. Z. S. Chheda, G. Kohanbash, K. Okada, N. Jahan, J. Sidney, M. Pecoraro, X. Yang, D. A. Carrera, K. M. Downey, S. Shrivastav, S. Liu, Y. Lin, C. Lagisetti, P. Chuntova, P. B. Watchmaker, S. Mueller, I. F. Pollack, R. Rajalingam, A. M. Carcaboso, M. Mann, A. Sette, K. C. Garcia, Y. Hou, H. Okada, Novel and shared neoantigen derived from histone 3 variant H3.3K27M mutation for glioma T cell therapy. J Exp Med 215, 141-157 (2018).
32. C. V. Jakubzick, G. J. Randolph, P. M. Henson, Monocyte differentiation and antigen-presenting functions. Nat Rev Immunol 17, 349-362 (2017).
33. T. L. Tang-Huau, P. Gueguen, C. Goudot, M. Durand, M. Bohec, S. Baulande, B. Pasquier, S. Amigorena, E. Segura, Human in vivo-generated monocyte-derived dendritic cells and macrophages cross-present antigens through a vacuolar pathway. Nat Commun 9, 2570 (2018).
34. M. Guilliams, F. Ginhoux, C. Jakubzick, S. H. Naik, N. Onai, B. U. Schraml, E. Segura, R. Tussiwand, S. Yona, Dendritic cells, monocytes and macrophages: a unified nomenclature based on ontogeny. Nat Rev Immunol 14, 571-578 (2014).
35. A. Mackay, A. Burford, V. Molinari, D. T. W. Jones, E. Izquierdo, J. Brouwer-Visser, F. Giangaspero, C. Haberler, T. Pietsch, T. S. Jacques, D. Figarella-Branger, D. Rodriguez, P. S. Morgan, P. Raman, A. J. Waanders, A. C. Resnick, M. Massimino, M. L. Garre, H. Smith, D. Capper, S. M. Pfister, T. Wurdinger, R. Tam, J. Garcia, M. D. Thakur, G. Vassal, J. Grill, T. Jaspan, P. Varlet, C. Jones, Molecular, Pathological, Radiological, and Immune Profiling of Non-brainstem Pediatric High-Grade Glioma from the HERBY Phase II Randomized Trial. Cancer Cell 33, 829-842 e825 (2018).
36. S. Mueller, J. M. Taitt, J. E. Villanueva-Meyer, E. R. Bonner, T. Nejo, R. R. Lulla, S. Goldman, A. Banerjee, S. N. Chi, N. S. Whipple, J. R. Crawford, K. Gauvain, K. J. Nazemi, P. B. Watchmaker, N. D. Almeida, K. Okada, A. M. Salazar, R. D. Gilbert, J. Nazarian, A. M. Molinaro, L. H. Butterfield, M. D. Prados, H. Okada, Mass cytometry detects H3.3K27M-specific vaccine responses in diffuse midline glioma. J Clin Invest 130, 6325-6337 (2020).
37. Y. Sangatsuda, F. Miura, H. Araki, M. Mizoguchi, N. Hata, D. Kuga, R. Hatac, Y. Akagi, T. Amemiya, Y. Fujioka, Y. Arai, A. Yoshida, T. Shibata, K. Yoshimoto, K. Jihara, T. Ito, Base-resolution methylomes of gliomas bearing histone H3.3 mutations reveal a G34 mutant-specific signature shared with bone tumors. Sci Rep 10, 16162 (2020).
38. C. C. L. Chen, S. Deshmukh, S. Jessa, D. Hadjadj, V. Lisi, A. F. Andrade, D. Faury, W. Jawhar, R. Dali, H. Suzuki, M. Pathania, D. A, F. Dubois, E. Woodward, S. Hebert, M. Coutelier, J. Karamchandani, S. Albrecht, S. Brandner, N. De Jay, T. Gayden, A. Bajic, A. S. Harutyunyan, D. M. Marchione, L. G. Mikael, N. Juretic, M. Zeinich, C. Russo, N. Maestro, A. V. Bassenden, P. Hauser, J. Virga, L. Bognar, A. Klekner, M. Zapotocky, A. Vicha, L. Krskova, K. Vanova, J. Zamecnik, D. Sumerauer, P. G. Ekert, D. S. Ziegler, B. Ellezam, M. G. Filbin, M. Blanchette, J. R. Hansford, D. A. Khuong-Quang, A. M. Berghuis, A. G. Weil, B. A. Garcia, L. Garzia, S. C. Mack, R. Beroukhim, K. L. Ligon, M. D. Taylor, P. Bandopadhayay, C. Kramm, S. M. Pfister, A. Korshunov, D. Sturm, D. T. W. Jones, P. Salomoni, C. L. Kleinman, N. Jabado, Histone H3.3G34-Mutant Interneuron Progenitors Co-opt PDGFRA for Gliomagenesis. Cell 183, 1617-1633 e1622 (2020).
39. H. Chapman, A. Riesenberg, L. A. Ehrman, V. Kohli, D. Nardini, M. Nakafuku, K. Campbell, R. R. Waclaw, Gsx transcription factors control neuronal versus glial specification in ventricular zone progenitors of the mouse lateral ganglionic eminence. Dev Biol 442, 115-126 (2018).
40. A. Kallies, D. Zehn, D. T. Utzschneider, Precursor exhausted T cells: key to successful immunotherapy? Nat Rev Immunol 20, 128-136 (2020).
41. M. Saillard, M. Cenerenti, P. Romero, C. Jandus, Impact of Immunotherapy on CD4 T Cell Phenotypes and Function in Cancer. Vaccines (Basel) 9, (2021).
42. J. R. Richardson, A. Schollhorn, C. Gouttefangeas, J. Schuhmacher, CD4+ T Cells: Multitasking Cells in the Duty of Cancer Immunotherapy. Cancers (Basel) 13, (2021).
43. R. E. Tay, E. K. Richardson, H. C. Toh, Revisiting the role of CD4 (+) T cells in cancer immunotherapy-new insights into old paradigms. Cancer Gene Ther 28, 5-17 (2021).
44. C. Rabu, L. Rangan, L. Florenceau, A. Fortun, M. Charpentier, E. Dupre, L. Paolini, C. Beauvillain, E. Dupel, J. B. Latouche, O. Adotevi, N. Labarriere, F. Lang, Cancer vaccines: designing artificial synthetic long peptides to improve presentation of class I and class II T cell epitopes by dendritic cells. Oncoimmunology 8, e1560919 (2019).
45. A. M. Swartz, K. L. Congdon, S. K. Nair, Q. J. Li, J. E. Herndon, 2nd, C. M. Suryadevara, K. A. Riccione, G. E. Archer, P. K. Norberg, L. A. Sanchez-Perez, J. H. Sampson, A conjoined universal helper epitope can unveil antitumor effects of a neoantigen vaccine targeting an MHC class I-restricted neoepitope. NPJ Vaccines 6, 12 (2021).
46. E. Segura, S. Amigorena, Inflammatory dendritic cells in mice and humans. Trends Immunol 34, 440-445 (2013).
47. E. Tcyganov, J. Mastio, E. Chen, D. I. Gabrilovich, Plasticity of myeloid-derived suppressor cells in cancer. Curr Opin Immunol 51, 76-82 (2018).
48. A. R. Pombo Antunes, I. Scheyltjens, F. Lodi, J. Messiaen, A. Antoranz, J. Duerinck, D. Kancheva, L. Martens, K. De Vlaminck, H. Van Hove, S. S. Kjolner Hansen, F. M. Bosisio, K. Van der Borght, S. De Vleeschouwer, R. Sciot, L. Bouwens, M. Verfaillie, N. Vandamme, R. E. Vandenbroucke, O. De Wever, Y. Saeys, M. Guilliams, C. Gysemans, B. Neyns, F. De Smet, D. Lambrechts, J. A. Van Ginderachter, K. Movahedi, Single-cell profiling of myeloid cells in glioblastoma across species and disease stage reveals macrophage competition and specialization. Nat Neurosci 24, 595-610 (2021).
49. A. Bachem, E. Hartung, S. Guttler, A. Mora, X. Zhou, A. Hegemann, M. Plantinga, E. Mazzini, P. Stoitzner, S. Gurka, V. Henn, H. W. Mages, R. A. Kroczek, Expression of XCR1 Characterizes the Batf3-Dependent Lineage of Dendritic Cells Capable of Antigen Cross-Presentation. Front Immunol 3, 214 (2012).
50. Y. Willemen, M. Versteven, M. Peeters, Z. N. Berneman, E. L. J. Smits, Ribonucleic Acid Engineering of Dendritic Cells for Therapeutic Vaccination: Ready 'N Able to Improve Clinical Outcome? Cancers (Basel) 12, (2020).
51. M. H. Meel, A. C. P. Sewing, P. Waranecki, D. S. Metselaar, L. E. Wedekind, J. Koster, D. G. van Vuurden, G. J. L. Kaspers, E. Hulleman, Culture methods of diffuse intrinsic pontine glioma cells determine response to targeted therapies. Exp Cell Res 360, 397-403 (2017).
52. D. N. Lisiero, H. Soto, L. M. Liau, R. M. Prins, Enhanced sensitivity to IL-2 signaling regulates the clinical responsiveness of IL-12-primed CD8 (+) T cells in a melanoma model. J Immunol 186, 5068-5077 (2011).
53. S. Shen, J. W. Park, Z. X. Lu, L. Lin, M. D. Henry, Y. N. Wu, Q. Zhou, Y. Xing, rMATS: robust and flexible detection of differential alternative splicing from replicate RNA-Seq data. Proc Natl Acad Sci USA 111, E5593-5601 (2014).
54. L. W. Nyambura, A. A. Munoz, P. le Coutre, P. Walden, HLA class I-restricted T cell epitopes isolated and identified from myeloid leukemia cells. Sci Rep 9, 14029 (2019).

1. A peptide comprising:
(i) at least 70% sequence identity to a peptide of one of SEQ ID NOS: 1-107; or
(ii) at least 6 contiguous amino acids from a peptide of one of SEQ ID NOS: 1-107, wherein the peptide comprises an alternative splice site junction.

2-13. (canceled)

14. A nucleic acid encoding for the peptide of claim 1.

15-16. (canceled)

17. An expression vector comprising the nucleic acid of claim 14.

18. A molecular complex comprising the peptide of claim 1 and a MHC polypeptide.

19-27. (canceled)

28. A host cell comprising the nucleic acid of claim 14.

29. An in vitro isolated dendritic cell comprising the peptide of claim 1.

30-36. (canceled)

37. A method of making a cell comprising transferring the nucleic acid of claim 14 into the cell.

38-45. (canceled)

46. A method of producing glioma-specific immune effector cells comprising:
(a) obtaining a starting population of immune effector cells; and
(b) contacting the starting population of immune effector cells with a peptide of claim 1, thereby generating peptide-specific immune effector cells.

47-75. (canceled)

76. A peptide-specific engineered T cell produced according to any one of claim 46.

77. (canceled)

78. A method of treating or preventing gliomas in a subject or for stimulating an immune response in a subject, the method comprising administering an effective amount of the dendritic cell of claim 29 to the subject.

79-80. (canceled)

81. The method of claim 78, wherein the glioma comprises high-grade glioma.

82. (canceled)

83. The method of claim 78, wherein the subject has a H3G34R/V and/or H3K27M mutation in the histone H3 gene.

84. The method of claim 78, wherein the subject is a human.

85. (canceled)

86. The method of claim 78, further comprising administering an anti-cancer agent, wherein the anti-cancer treatment comprises one or more of surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

87-92. (canceled)

93. The method of claim 86, wherein the anti-cancer agent comprises anti-PD1 monoclonal antibody monotherapy.

94-96. (canceled)

97. The method of claim 78, wherein the cancer comprises a glioma that is positive for expression of the peptide.

98. (canceled)

99. A method for prognosing a patient or for detecting T cell responses in a patient, the method comprising: contacting a biological sample from the patient with the peptide of claim 1.

100-107. (canceled)

108. A composition comprising at least one MHC polypeptide and the peptide of claim 1.

109-115. (canceled)

116. A method comprising contacting the composition of claim 109 with a composition comprising T cells and detecting T cells with bound peptide and/or MHC polypeptide by detecting a detection tag.

117-123. (canceled)

124. A kit comprising the peptide of claim 1 in a container.

125-127. (canceled)
